SEQUENCE LISTING (64 sequences)

SEQ ID NO: 1 - DNA, 7483 bp, Homo sapiens
agtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccagaactgtgcctggcccagtgggcagcaggagctcctgacttgggaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaacagtcacggggaggtgctggttccatgcctgctctcgaggcagcagtgggttcaggcccatcagctacccctgcagctggggaagacttatgccatcccggcagcgaggctgggctggccagccaccactgactataccaactgggcctctgatgttcttccagtgaggcatctctctgggatgcagaacttccctccatccacccctctggcacctgggttggtctaatcctagtttgctgtggccttcccggttgtgagagcctgtgatccttagatgtgtctcctgtttcagaccagccccaccatgcaacttcctttgactttctgtgtaccactgggatagaggaatcaagaggacaatctagctctccatactttgaacaaccaaatgtgcattgaatactctgaaaccgaagggactggatctgcaggtgggatgagggagacagaccacttttctatattgcagtgtgaatgctgggcccctgctcaagtctaccctgatcacctcagggcataaagcatgtttcattctctggcc

SEQ ID NO: 2 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 3 - DNA, 7363 bp, Homo sapiens
agtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccagaactgtgcctggcccagtgggcagcaggagctcctgacttgggaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaacagtcacggggaggtgctggttccatgcctgctctcgaggcagcagtgggttcaggcccatcagctacccctgcagctggggaagacttatgccatcccggcagcgaggctgggctggccagccaccactgactataccaactgggcctctgatgttcttccagtgaggcatctctctgggatgcagaacttccctccatccacccctctggcacctgggttggtctaatcctagtttgctgtggccttcccggttgtgagagcctgtgatccttagatgtgtctcctgtttcagaccagccccaccatgcaacttcctttgactttctgtgtaccactgggatagaggaatcaagaggacaatctagctctccatactttgaacaaccaaatgtgcattgaatactctgaaaccgaagggactggatctgcaggtgggatgagggagacagaccacttttctatattgcagtgtgaatgctgggcccctgctcaagtctaccctgatcacctcagggcataaagcatgtttcattctctggcc

SEQ ID NO: 4 - protein, 2175 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 5 - DNA, 7449 bp, Homo sapiens
agtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccagaactgtgcctggcccagtgggcagcaggagctcctgacttgggaccatggtgattcttcagcaggagaaaagctgggacttgccccacgttacacagccacactgaggcatgtgtcgtgtcacaaacgagatcttccattcaagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaacagtcacggggaggtgctggttccatgcctgctctcgaggcagcagtgggttcaggcccatcagctacccctgcagctggggaagacttatgccatcccggcagcgaggctgggctggccagccaccactgactataccaactgggcctctgatgttcttccagtgaggcatctctctgggatgcagaacttccctccatccacccctctggcacctgggttggtctaatcctagtttgctgtggccttcccggttgtgagagcctgtgatccttagatgtgtctcctgtttcagaccagccccaccatgcaacttcctttgactttctgtgtaccactgggatagaggaatcaagaggacaatctagctctccatactttgaacaaccaaatgtgcattgaatactctgaaaccgaagggactggatctgcaggtgggatgagggagacagaccacttttctatattgcagtgtgaatgctgggcccctgctcaagtctaccctgatcacctcagggcataaagcatgtttcattctctggcc

SEQ ID NO: 6 - protein, 2166 aa, Homo sapiens
MDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 7 - DNA, 7481 bp, Mus musculus
agtgcaggctggacagctgccctgaacagaaagaaagagtgacccagggagacaagaaacagagtagcccaagggaagcccacagcagcagcagatcaaggctcaagctggagctgaaaatttgcaggctccagcctcagcttccagagtcctcctgacctgtgacccctggctcctggctgggaggtggtgactcggagggtgtggataaaacccagagctgtgtctggtcactccggcaggtgtgctgacgtagaagcatggttattctgcagaagggggactatgtatggatggacctgaagtcaggccaggagtttgatgtgcccatcggggccgtggtgaagctctgcgactcgggccagatccaggtggtggatgatgaagacaatgaacactggatatcccctcagaatgccacgcacatcaagccaatgcaccccacatcggtgcacggcgtggaggacatgatccgcctgggggatctcaacgaggcaggcatccttcgaaaccttctcattcgctaccgggaccacctcatctatacgtacacaggttccatcctggtggccgtgaacccctaccagctgctctccatctactcgccagagcacatccgccagtacaccaacaagaagataggggagatgcccccccacatcttcgccattgctgacaactgctacttcaacatgaaacgcaacaaccgggaccagtgctgtattatcagcggggagtcgggagctggcaagacagagagcacaaagttgatcctgcagttcctggcagccatcagtggacagcactcatggatcgagcagcaggtgctggaggccaccccgatcctggaagcatttgggaacgccaagaccatccgcaacgacaactctagccgctttggcaagtacattgacatccactttaacaagcgtggtgccatcgagggcgccaaaatagagcaatacctgctggagaagtcacgtgtctgccgccaggcccctgacgagaggaactatcacgtgttctactgtatgctggagggcatgaatgaggaggagaagaagaaactgggcctaggccaggccgctgactacaactacttggccatgggtaactgcatcacctgtgagggccgcgtggacagtcaggagtatgccaacatccgctctgccatgaaggttctcatgttcacagacacggagaactgggagatctcgaagcttctggctgccatcctacacatgggcaatctgcagtatgaggcccggacatttgagaacttggatgcgtgtgaagtcctcttctccccatcgctggccacggcagcttctctgctcgaggtgaaccccccagacctgatgagctgcctcaccagccgcaccctcatcacccgtggggagacggtgtccacccctctcagcagggaacaggcgctggatgtgcgagatgcctttgtcaagggcatctatgggcggctctttgtgtggattgtggagaagatcaacgcagcaatctacaagccaccccccctggaagtgaagaactctcgccggtccatcggtctcctggacatctttggatttgagaacttcactgtgaacagcttcgagcagctctgcattaactttgccaatgagcacctgcagcaattcttcgtgcggcacgtgttcaagctggagcaggaggagtacgacctggagagcatcgactggttgcacattgagttcactgacaaccaggaagcactggacatgattgccaaccggcctatgaacgtcatctccctcatcgatgaggagagcaagttccccaagggcacggatgccaccatgctgcataagctgaactcacagcacaagctcaatgccaactacgtgccacccaagaacagccacgagacccagtttggaatcaaccactttgcgggtgttgtctattatgagagtcaaggcttcctggagaagaaccgagacaccctgcatggggacatcatccagctggtccactcttcccggaacaagttcataaagcagattttccaagctgacgttgccatgggtgccgagaccaggaagcgctcgcctacactcagcagccagttcaagcggtctctggagctgctgatgcgcacactgggcgcctgccagcccttctttgtgcgttgtatcaaacccaatgagttcaagaagcccatgctcttcgaccggcacttgtgtgtacgccagctgcgatattcgggcatgatggagacaatccgcatccgccacgcaggctaccccattcgctacagctttgtggagtttgtggagcgctaccgggtactgctgcctggtgtgaagccagcatacaagcagggtgacctccgagggacatgccagcgcatggctgaggctgtgctgggcacgcacgatgactggcagattggcaaaaccaagatctttctgaaggaccaccatgacatgttgctggaggtggagcgggacaaggccatcacagacagagtcattctcctccagaaggttatccggggcttcaaagacaggtccaacttcctgagactgaagagtgctgccacactgatccagaggcactggcggggccaccactgtaggaaaaactatgagctgattcgtcttggcttcctgcggctgcaggccctgcaccgctcccggaagctgcacaagcagtaccgcctggccagacagcgcataatagagttccaggcccgctgccgggcctatctggtgcgcaaggccttccgccaccgcctctgggccgtgatcaccgtgcaggcctatgcccgaggcatgattgcccgccggctacaccgccgcctccgggttgagtaccagcggcgcctcgaggcagagaggatgcgtctggcagaggaggagaaactccgaaaggagatgagtgccaagaaggccaaagaggaggctgagcgcaagcatcaggagcgcctggctcagctagcccgcgaggatgcggagcgggaactgaaggagaaggaggaggctcggaggaagaaggaactgctggagcagatggagaaggcccgccacgaacccatcaaccactcagatatggtggacaagatgtttggcttcctggggacttcaggcagcctgccaggccaggaaggccaggcgcctagtggctttgaggacctagagcgcggacggagggagatggtggaagaggatgttgacgctgccctgcccctgcctgatgaagacgaggaggacctttctgagtacaaattcgccaagtttgctgccacctacttccagggcacaaccacacactcctacacccggaggcctctcaagcagccgctgctctaccacgacgatgagggtgaccagctggcggcgctggctgtctggatcaccatcctccggttcatgggggacctcccagagcccaagtaccacacagccatgagcgacggcagtgagaagatcccagtgatgactaagatctacgagaccctaggcaagaagacatataagagggagctgcaggccttgcagggcgagggcgagacccagctccctgaggggcagaagaagaccagtgtgagacacaagttggtacacttgacactgaagaaaaagtccaaactcacagaagaggtgaccaagaggctgaacgatggggaatccacggtacagggcaacagcatgctggaggatcggcccacctcaaatctagagaagctgcacttcatcatcggcaacggcatcctgcggcctgcgctgcgggacgagatttactgccagatcagtaagcagctcacacacaacccatccaagagcagctatgccaggggctggatcctcgtgtcgctctgtgtgggctgcttcgccccctctgagaagttcgttaagtacctgcggaacttcatccacggaggcccacctggctatgctccttactgtgaggagcgcctgaggaggacctttgtcaacggaactcggacacagccacccagctggctggagctgcaggccaccaagtccaagaagcccatcatgttgcccgtgaccttcatggatgggaccaccaagaccctgctaacagattcagcaactacagccagggagctgtgcaatgctctggctgacaagatctcactcaaggaccgctttggcttctccctctacatcgctctgttcgataaggtgtcctccctgggcagcggcagtgaccatgtcatggatgccatctctcagtgtgagcagtacgccaaggagcagggtgctcaggagcgcaacgccccatggaggctcttctttagaaaggaggtcttcacaccctggcacaacccctcggaggacaacgtggccacgaacctcatctaccagcaggtggtgcgaggagtcaagtttggggagtacaggtgtgagaaggaggacgacctggctgagctggcttctcagcagtactttgtggactatggttctgagatgattctggagcgcctgctgagcctcgtgcccacttacatccctgaccgtgagatcacaccgctgaagaatcttgagaagtgggcacagctggccattgctgcccacaagaagggaatttatgcccagaggagaactgactcccagaaggtcaaagaggatgtggtcaattatgcccgtttcaagtggcccttgctcttctccaggttttacgaagcttacaaattctcaggccctcccctccccaagagcgacgtcatcgtggctgtcaactggacgggtgtgtacttcgtggacgagcaggagcaggtgcttctggagctgtccttcccggagatcatggctgtgtccagcagtagggagtgccgcgtcttgctctcactgggctgctctgacttgggctgtgctacttgtcaatcgggccgggcagggctgaccccggctggaccctgttctccgtgttggtcctgtaggggaacaaagatgatggcccccagctttaccctggccaccatcaaaggagatgagtacaccttcacatccagcaatgctgaggacatccgtgacctggtggtcacctttctggaggggctacggaagaggtctaagtatgtggtggcactgcaggacaatcctaaccctgctggtgaggagtcaggcttcctcagcttcgccaagggagacctcatcatccttgaccatgatactggtgagcaggtcatgaactcaggctgggccaacggcatcaacgagaggaccaagcagcgcggcgacttccccactgactgtgtatacgtcatgcccactgtcaccttgccaccaagggagattgtggccctggtcactatgaccccagaccagaggcaggatgtcgtccggctcctgcagcttcgcacagcagagccagaggtgcgcgccaagccctacacgctagaggagttctcctacgactacttcaggcccccacccaagcacacgctgagccgtgtcatggtgtccaaggcccgcggtaaggacaggctgtggagccacacacgagagcccctcaagcaggccctgctcaagaagatcctgggcagtgaagaactctcccaggaagcctgcatggcctttgtagctgtgctcaagtacatgggcgactacccatccaagaggatgcgatccgtcaatgagctcactgaccagatctttgagtgggcactcaaggctgagcccctcaaggatgaggcctacgtgcagatcctgaagcagctgactgacaatcacatcaggtacagcgaagagaggggctgggaactgctgtggctgtgcacgggcctcttcccgcccagcaacatcctcctgcctcatgttcagcggtttctgcagtcccgcaagcactgtcctcttgccattgactgcctgcagaggctccagaaagccctgagaaatggctcccggaagtaccctccgcacctggtggaggtggaggccatccaacataagactacccagatcttccacaaggtctacttccccgatgacacggacgaggcttttgaggtggagtccagcaccaaggccaaggacttctgccagaacatcgccagccggctgctgctcaagtcttccgagggattcagcctttttgtcaaaatcgcagataaggtcatcagcgtcccagagaatgatttcttctttgactttgtccgacacctgacagactggataaagaaagcacggcccatcaaggacggaatcgtgccctcactaacctaccaggtgttcttcatgaagaagctgtggaccaccacagtgccgggcaaggaccccatggctgactccatcttccactattaccaggaactgcccaaatatctccgaggctaccacaagtgcacccgggaggaggtgctgcagctgggcgcactcatctacagggtcaagtttgaggaggacaaatcctacttccctagcatccccaagttgctgagggagctggtaccccaggacctaatccggcaggtctcacctgatgactggaaacggtctattgtcgcctacttcaacaaacatgcggggaagtccaaggaggaagccaagctggccttcctcaaactcatcttcaagtggcccacctttggctcagccttctttgaggtgaagcaaactacagaaccaaacttcccagagattctcttaattgccatcaacaagtacggggtcagcctcatcgatcccagaaccaaggacatcctgactactcaccccttcaccaagatctccaactggagtagtggcaacacctacttccacatcaccattgggaacttggtccgtgggagcaaactgctctgtgagacatcgctgggatacaaaatggatgatcttctgacttcctacatcagccagatgctcacagccatgagcaagcagaggaactccaggagtggaaggtgaacctcagaggagacgctggctcaggccttggccctctaggcagggaagctggactgaccatatctgctgggcatctgatctgcctgccacgaggtccagactcttctgcatccacctatggcatctgggtttgctgtcaccctactttgttgtggccttcctggtgtaagagtctgtgttccttggtcacctctcctgattcagaccagctccatcaagcaacctcttttgactttctgtatatggatggcacagaggaatcaaggacaacttagctctctgcatacttggaacaaccaaactatttgtacattgaacggatgctctgaaacccaagggactgggctcagggtcctcagcactggcccctgtcataagcactaccactaaggactctctggaggactcctcagtatcatctgctccaggaagccccctagactacctcctgagtctggacaaagcctcctgattctacctggatcactcctgttatgtgacagttatgtggtgggtccctgctaaaatctccctgaccacctgagggcataaagcatgtgtcttattctctgg

SEQ ID NO: 8 - protein, 2215 aa, Mus musculus
MVILQKGDYVWMDLKSGQEFDVPIGAVVKLCDSGQIQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNNRDQCCIISGESGAGKTESTKLILQFLAAISGQHSWIEQQVLEATPILEAFGNAKTIRNDNSSRFGKYIDIHFNKRGAIEGAKIEQYLLEKSRVCRQAPDERNYHVFYCMLEGMNEEEKKKLGLGQAADYNYLAMGNCITCEGRVDSQEYANIRSAMKVLMFTDTENWEISKLLAAILHMGNLQYEARTFENLDACEVLFSPSLATAASLLEVNPPDLMSCLTSRTLITRGETVSTPLSREQALDVRDAFVKGIYGRLFVWIVEKINAAIYKPPPLEVKNSRRSIGLLDIFGFENFTVNSFEQLCINFANEHLQQFFVRHVFKLEQEEYDLESIDWLHIEFTDNQEALDMIANRPMNVISLIDEESKFPKGTDATMLHKLNSQHKLNANYVPPKNSHETQFGINHFAGVVYYESQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRHAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLRLKSAATLIQRHWRGHHCRKNYELIRLGFLRLQALHRSRKLHKQYRLARQRIIEFQARCRAYLVRKAFRHRLWAVITVQAYARGMIARRLHRRLRVEYQRRLEAERMRLAEEEKLRKEMSAKKAKEEAERKHQERLAQLAREDAERELKEKEEARRKKELLEQMEKARHEPINHSDMVDKMFGFLGTSGSLPGQEGQAPSGFEDLERGRREMVEEDVDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGETQLPEGQKKTSVRHKLVHLTLKKKSKLTEEVTKRLNDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTARELCNALADKISLKDRFGFSLYIALFDKVSSLGSGSDHVMDAISQCEQYAKEQGAQERNAPWRLFFRKEVFTPWHNPSEDNVATNLIYQQVVRGVKFGEYRCEKEDDLAELASQQYFVDYGSEMILERLLSLVPTYIPDREITPLKNLEKWAQLAIAAHKKGIYAQRRTDSQKVKEDVVNYARFKWPLLFSRFYEAYKFSGPPLPKSDVIVAVNWTGVYFVDEQEQVLLELSFPEIMAVSSSRECRVLLSLGCSDLGCATCQSGRAGLTPAGPCSPCWSCRGTKMMAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDCVYVMPTVTLPPREIVALVTMTPDQRQDVVRLLQLRTAEPEVRAKPYTLEEFSYDYFRPPPKHTLSRVMVSKARGKDRLWSHTREPLKQALLKKILGSEELSQEACMAFVAVLKYMGDYPSKRMRSVNELTDQIFEWALKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIASRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPRTKDILTTHPFTKISNWSSGNTYFHITIGNLVRGSKLLCETSLGYKMDDLLTSYISQMLTAMSKQRNSRSGR

SEQ ID NO: 9 - DNA, 7214 bp, Mus musculus
ggcgctaggctgtgagatcatcagaggactccgttacccacacagctgccagaggggagttgtcaaagtgaaaccccagggactgggggtgtagtttgtggggtcagggggactatgtatggatggacctgaagtcaggccaggagtttgatgtgcccatcggggccgtggtgaagctctgcgactcgggccagatccaggtggtggatgatgaagacaatgaacactggatatcccctcagaatgccacgcacatcaagccaatgcaccccacatcggtgcacggcgtggaggacatgatccgcctgggggatctcaacgaggcaggcatccttcgaaaccttctcattcgctaccgggaccacctcatctataccagctgtggaggacggacgtacacaggttccatcctggtggccgtgaacccctaccagctgctctccatctactcgccagagcacatccgccagtacaccaacaagaagataggggagatgcccccccacatcttcgccattgctgacaactgctacttcaacatgaaacgcaacaaccgggaccagtgctgtattatcagcggggagtcgggagctggcaagacagagagcacaaagttgatcctgcagttcctggcagccatcagtggacagcactcatggatcgagcagcaggtgctggaggccaccccgatcctggaagcatttgggaacgccaagaccatccgcaacgacaactctagccgctttggcaagtacattgacatccactttaacaagcgtggtgccatcgagggcgccaaaatagagcaatacctgctggagaagtcacgtgtctgccgccaggcccctgacgagaggaactatcacgtgttctactgtatgctggagggcatgaatgaggaggagaagaagaaactgggcctaggccaggccgctgactacaactacttggccatgggtaactgcatcacctgtgagggccgcgtggacagtcaggagtatgccaacatccgctctgccatgaaggttctcatgttcacagacacggagaactgggagatctcgaagcttctggctgccatcctacacatgggcaatctgcagtatgaggcccggacatttgagaacttggatgcgtgtgaagtcctcttctccccatcgctggccacggcagcttctctgctcgaggtgaaccccccagacctgatgagctgcctcaccagccgcaccctcatcacccgtggggagacggtgtccacccctctcagcagggaacaggcgctggatgtgcgagatgcctttgtcaagggcatctatgggcggctctttgtgtggattgtggagaagatcaacgcagcaatctacaagccaccccccctggaagtgaagaactctcgccggtccatcggtctcctggacatctttggatttgagaacttcactgtgaacagcttcgagcagctctgcattaactttgccaatgagcacctgcagcaattcttcgtgcggcacgtgttcaagctggagcaggaggagtacgacctggagagcatcgactggttgcacattgagttcactgacaaccaggaagcactggacatgattgccaaccggcctatgaacgtcatctccctcatcgatgaggagagcaagttccccaagggcacggatgccaccatgctgcataagctgaactcacagcacaagctcaatgccaactacgtgccacccaagaacagccacgagacccagtttggaatcaaccactttgcgggtgttgtctattatgagagtcaaggcttcctggagaagaaccgagacaccctgcatggggacatcatccagctggtccactcttcccggaacaagttcataaagcagattttccaagctgacgttgccatgggtgccgagaccaggaagcgctcgcctacactcagcagccagttcaagcggtctctggagctgctgatgcgcacactgggcgcctgccagcccttctttgtgcgttgtatcaaacccaatgagttcaagaagcccatgctcttcgaccggcacttgtgtgtacgccagctgcgatattcgggcatgatggagacaatccgcatccgccacgcaggctaccccattcgctacagctttgtggagtttgtggagcgctaccgggtactgctgcctggtgtgaagccagcatacaagcagggtgacctccgagggacatgccagcgcatggctgaggctgtgctgggcacgcacgatgactggcagattggcaaaaccaagatctttctgaaggaccaccatgacatgttgctggaggtggagcgggacaaggccatcacagacagagtcattctcctccagaaggttatccggggcttcaaagacaggtccaacttcctgagactgaagagtgctgccacactgatccagaggcactggcggggccaccactgtaggaaaaactatgagctgattcgtcttggcttcctgcggctgcaggccctgcaccgctcccggaagctgcacaagcagtaccgcctggccagacagcgcataatagagttccaggcccgctgccgggcctatctggtgcgcaaggccttccgccaccgcctctgggccgtgatcaccgtgcaggcctatgcccgaggcatgattgcccgccggctacaccgccgcctccgggttgagtaccagcggcgcctcgaggcagagaggatgcgtctggcagaggaggagaaactccgaaaggagatgagtgccaagaaggccaaagaggaggctgagcgcaagcatcaggagcgcctggctcagctagcccgcgaggatgcggagcgggaactgaaggagaaggaggaggctcggaggaagaaggaactgctggagcagatggagaaggcccgccacgaacccatcaaccactcagatatggtggacaagatgtttggcttcctggggacttcaggcagcctgccaggccaggaaggccaggcgcctagtggctttgaggacctagagcgcggacggagggagatggtggaagaggatgttgacgctgccctgcccctgcctgatgaagacgaggaggacctttctgagtacaaattcgccaagtttgctgccacctacttccagggcacaaccacacactcctacacccggaggcctctcaagcagccgctgctctaccacgacgatgagggtgaccagctggcggcgctggctgtctggatcaccatcctccggttcatgggggacctcccagagcccaagtaccacacagccatgagcgacggcagtgagaagatcccagtgatgactaagatctacgagaccctaggcaagaagacatataagagggagctgcaggccttgcagggcgagggcgagacccagctccctgaggggcagaagaagaccagtgtgagacacaagttggtacacttgacactgaagaaaaagtccaaactcacagaagaggtgaccaagaggctgaacgatggggaatccacggtacagggcaacagcatgctggaggatcggcccacctcaaatctagagaagctgcacttcatcatcggcaacggcatcctgcggcctgcgctgcgggacgagatttactgccagatcagtaagcagctcacacacaacccatccaagagcagctatgccaggggctggatcctcgtgtcgctctgtgtgggctgcttcgccccctctgagaagttcgttaagtacctgcggaacttcatccacggaggcccacctggctatgctccttactgtgaggagcgcctgaggaggacctttgtcaacggaactcggacacagccacccagctggctggagctgcaggccaccaagtccaagaagcccatcatgttgcccgtgaccttcatggatgggaccaccaagaccctgctaacagattcagcaactacagccagggagctgtgcaatgctctggctgacaagatctcactcaaggaccgctttggcttctccctctacatcgctctgttcgataaggtgtcctccctgggcagcggcagtgaccatgtcatggatgccatctctcagtgtgagcagtacgccaaggagcagggtgctcaggagcgcaacgccccatggaggctcttctttagaaaggaggtcttcacaccctggcacaacccctcggaggacaacgtggccacgaacctcatctaccagcaggtggtgcgaggagtcaagtttggggagtacaggtgtgagaaggaggacgacctggctgagctggcttctcagcagtactttgtggactatggttctgagatgattctggagcgcctgctgagcctcgtgcccacttacatccctgaccgtgagatcacaccgctgaagaatcttgagaagtgggcacagctggccattgctgcccacaagaagggaatttatgcccagaggagaactgactcccagaaggtcaaagaggatgtggtcaattatgcccgtttcaagtggcccttgctcttctccaggttttacgaagcttacaaattctcaggccctcccctccccaagagcgacgtcatcgtggctgtcaactggacgggtgtgtacttcgtggacgagcaggagcaggtgcttctggagctgtccttcccggagatcatggctgtgtccagcagtaggggaacaaagatgatggcccccagctttaccctggccaccatcaaaggagatgagtacaccttcacatccagcaatgctgaggacatccgtgacctggtggtcacctttctggaggggctacggaagaggtctaagtatgtggtggcactgcaggacaatcctaaccctgctggtgaggagtcaggcttcctcagcttcgccaagggagacctcatcatccttgaccatgatactggtgagcaggtcatgaactcaggctgggccaacggcatcaacgagaggaccaagcagcgcggcgacttccccactgactgtgtatacgtcatgcccactgtcaccttgccaccaagggagattgtggccctggtcactatgaccccagaccagaggcaggatgtcgtccggctcctgcagcttcgcacagcagagccagaggtgcgcgccaagccctacacgctagaggagttctcctacgactacttcaggcccccacccaagcacacgctgagccgtgtcatggtgtccaaggcccgcggtaaggacaggctgtggagccacacacgagagcccctcaagcaggccctgctcaagaagatcctgggcagtgaagaactctcccaggaagcctgcatggcctttgtagctgtgctcaagtacatgggcgactacccatccaagaggatgcgatccgtcaatgagctcactgaccagatctttgagtgggcactcaaggctgagcccctcaaggatgaggcctacgtgcagatcctgaagcagctgactgacaatcacatcaggtacagcgaagagaggggctgggaactgctgtggctgtgcacgggcctcttcccgcccagcaacatcctcctgcctcatgttcagcggtttctgcagtcccgcaagcactgtcctcttgccattgactgcctgcagaggctccagaaagccctgagaaatggctcccggaagtaccctccgcacctggtggaggtggaggccatccaacataagactacccagatcttccacaaggtctacttccccgatgacacggacgaggcttttgaggtggagtccagcaccaaggccaaggacttctgccagaacatcgccagccggctgctgctcaagtcttccgagggattcagcctttttgtcaaaatcgcagataaggtcatcagcgtcccagagaatgatttcttctttgactttgtccgacacctgacagactggataaagaaagcacggcccatcaaggacggaatcgtgccctcactaacctaccaggtgttcttcatgaagaagctgtggaccaccacagtgccgggcaaggaccccatggctgactccatcttccactattaccaggaactgcccaaatatctccgaggctaccacaagtgcacccgggaggaggtgctgcagctgggcgcactcatctacagggtcaagtttgaggaggacaaatcctacttccctagcatccccaagttgctgagggagctggtaccccaggacctaatccggcaggtctcacctgatgactggaaacggtctattgtcgcctacttcaacaaacatgcggggaagtccaaggaggaagccaagctggccttcctcaaactcatcttcaagtggcccacctttggctcagccttctttgaggtgaagcaaactacagaaccaaacttcccagagattctcttaattgccatcaacaagtacggggtcagcctcatcgatcccagaaccaaggacatcctgactactcaccccttcaccaagatctccaactggagtagtggcaacacctacttccacatcaccattgggaacttggtccgtgggagcaaactgctctgtgagacatcgctgggatacaaaatggatgatcttctgacttcctacatcagccagatgctcacagccatgagcaagcagaggaactccaggagtggaaggtgaacctcagaggagacgctggctcaggccttggccctctaggcagggaagctggactgaccatatctgctgggcatctgatctgcctgccacgaggtccagactcttctgcatccacctatggcatctgggtttgctgtcaccctactttgttgtggccttcctggtgtaagagtctgtgttccttggtcacctctcctgattcagaccagctccatcaagcaacctcttttgactttctgtatatggatggcacagaggaatcaaggacaacttagctctctgcatacttggaacaaccaaactatttgtacattgaacggatgctctgaaacccaagggactgggctcagggtcctcagcactggcccctgtcataagcactaccactaaggactctctggaggactcctcagtatcatctgctccaggaagccccctagactacctcctgagtctggacaaagcctcctgattctacctggatcactcctgttatgtgacagttatgtggtgggtccctgctaaaatctccctgaccacctgagggcataaagcatgtgtcttattctctgg

SEQ ID NO: 10 - protein, 2172 aa, Mus musculus
MDLKSGQEFDVPIGAVVKLCDSGQIQVVDDEDNEHWISPQNATHIKPMHPTSVHGVEDMIRLGDLNEAGILRNLLIRYRDHLIYTSCGGRTYTGSILVAVNPYQLLSIYSPEHIRQYTNKKIGEMPPHIFAIADNCYFNMKRNNRDQCCIISGESGAGKTESTKLILQFLAAISGQHSWIEQQVLEATPILEAFGNAKTIRNDNSSRFGKYIDIHFNKRGAIEGAKIEQYLLEKSRVCRQAPDERNYHVFYCMLEGMNEEEKKKLGLGQAADYNYLAMGNCITCEGRVDSQEYANIRSAMKVLMFTDTENWEISKLLAAILHMGNLQYEARTFENLDACEVLFSPSLATAASLLEVNPPDLMSCLTSRTLITRGETVSTPLSREQALDVRDAFVKGIYGRLFVWIVEKINAAIYKPPPLEVKNSRRSIGLLDIFGFENFTVNSFEQLCINFANEHLQQFFVRHVFKLEQEEYDLESIDWLHIEFTDNQEALDMIANRPMNVISLIDEESKFPKGTDATMLHKLNSQHKLNANYVPPKNSHETQFGINHFAGVVYYESQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRHAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLRLKSAATLIQRHWRGHHCRKNYELIRLGFLRLQALHRSRKLHKQYRLARQRIIEFQARCRAYLVRKAFRHRLWAVITVQAYARGMIARRLHRRLRVEYQRRLEAERMRLAEEEKLRKEMSAKKAKEEAERKHQERLAQLAREDAERELKEKEEARRKKELLEQMEKARHEPINHSDMVDKMFGFLGTSGSLPGQEGQAPSGFEDLERGRREMVEEDVDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGETQLPEGQKKTSVRHKLVHLTLKKKSKLTEEVTKRLNDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTARELCNALADKISLKDRFGFSLYIALFDKVSSLGSGSDHVMDAISQCEQYAKEQGAQERNAPWRLFFRKEVFTPWHNPSEDNVATNLIYQQVVRGVKFGEYRCEKEDDLAELASQQYFVDYGSEMILERLLSLVPTYIPDREITPLKNLEKWAQLAIAAHKKGIYAQRRTDSQKVKEDVVNYARFKWPLLFSRFYEAYKFSGPPLPKSDVIVAVNWTGVYFVDEQEQVLLELSFPEIMAVSSSRGTKMMAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDCVYVMPTVTLPPREIVALVTMTPDQRQDVVRLLQLRTAEPEVRAKPYTLEEFSYDYFRPPPKHTLSRVMVSKARGKDRLWSHTREPLKQALLKKILGSEELSQEACMAFVAVLKYMGDYPSKRMRSVNELTDQIFEWALKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIASRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPRTKDILTTHPFTKISNWSSGNTYFHITIGNLVRGSKLLCETSLGYKMDDLLTSYISQMLTAMSKQRNSRSGR

SEQ ID NO: 11 - DNA, 7196 bp, Mus musculus
ggcgctaggctgtgagatcatcagaggactccgttacccacacagctgccagaggggagttgtcaaagtgaaaccccagggactgggggtgtagtttgtggggtcagggggactatgtatggatggacctgaagtcaggccaggagtttgatgtgcccatcggggccgtggtgaagctctgcgactcgggccagatccaggtggtggatgatgaagacaatgaacactggatatcccctcagaatgccacgcacatcaagccaatgcaccccacatcggtgcacggcgtggaggacatgatccgcctgggggatctcaacgaggcaggcatccttcgaaaccttctcattcgctaccgggaccacctcatctatacgtacacaggttccatcctggtggccgtgaacccctaccagctgctctccatctactcgccagagcacatccgccagtacaccaacaagaagataggggagatgcccccccacatcttcgccattgctgacaactgctacttcaacatgaaacgcaacaaccgggaccagtgctgtattatcagcggggagtcgggagctggcaagacagagagcacaaagttgatcctgcagttcctggcagccatcagtggacagcactcatggatcgagcagcaggtgctggaggccaccccgatcctggaagcatttgggaacgccaagaccatccgcaacgacaactctagccgctttggcaagtacattgacatccactttaacaagcgtggtgccatcgagggcgccaaaatagagcaatacctgctggagaagtcacgtgtctgccgccaggcccctgacgagaggaactatcacgtgttctactgtatgctggagggcatgaatgaggaggagaagaagaaactgggcctaggccaggccgctgactacaactacttggccatgggtaactgcatcacctgtgagggccgcgtggacagtcaggagtatgccaacatccgctctgccatgaaggttctcatgttcacagacacggagaactgggagatctcgaagcttctggctgccatcctacacatgggcaatctgcagtatgaggcccggacatttgagaacttggatgcgtgtgaagtcctcttctccccatcgctggccacggcagcttctctgctcgaggtgaaccccccagacctgatgagctgcctcaccagccgcaccctcatcacccgtggggagacggtgtccacccctctcagcagggaacaggcgctggatgtgcgagatgcctttgtcaagggcatctatgggcggctctttgtgtggattgtggagaagatcaacgcagcaatctacaagccaccccccctggaagtgaagaactctcgccggtccatcggtctcctggacatctttggatttgagaacttcactgtgaacagcttcgagcagctctgcattaactttgccaatgagcacctgcagcaattcttcgtgcggcacgtgttcaagctggagcaggaggagtacgacctggagagcatcgactggttgcacattgagttcactgacaaccaggaagcactggacatgattgccaaccggcctatgaacgtcatctccctcatcgatgaggagagcaagttccccaagggcacggatgccaccatgctgcataagctgaactcacagcacaagctcaatgccaactacgtgccacccaagaacagccacgagacccagtttggaatcaaccactttgcgggtgttgtctattatgagagtcaaggcttcctggagaagaaccgagacaccctgcatggggacatcatccagctggtccactcttcccggaacaagttcataaagcagattttccaagctgacgttgccatgggtgccgagaccaggaagcgctcgcctacactcagcagccagttcaagcggtctctggagctgctgatgcgcacactgggcgcctgccagcccttctttgtgcgttgtatcaaacccaatgagttcaagaagcccatgctcttcgaccggcacttgtgtgtacgccagctgcgatattcgggcatgatggagacaatccgcatccgccacgcaggctaccccattcgctacagctttgtggagtttgtggagcgctaccgggtactgctgcctggtgtgaagccagcatacaagcagggtgacctccgagggacatgccagcgcatggctgaggctgtgctgggcacgcacgatgactggcagattggcaaaaccaagatctttctgaaggaccaccatgacatgttgctggaggtggagcgggacaaggccatcacagacagagtcattctcctccagaaggttatccggggcttcaaagacaggtccaacttcctgagactgaagagtgctgccacactgatccagaggcactggcggggccaccactgtaggaaaaactatgagctgattcgtcttggcttcctgcggctgcaggccctgcaccgctcccggaagctgcacaagcagtaccgcctggccagacagcgcataatagagttccaggcccgctgccgggcctatctggtgcgcaaggccttccgccaccgcctctgggccgtgatcaccgtgcaggcctatgcccgaggcatgattgcccgccggctacaccgccgcctccgggttgagtaccagcggcgcctcgaggcagagaggatgcgtctggcagaggaggagaaactccgaaaggagatgagtgccaagaaggccaaagaggaggctgagcgcaagcatcaggagcgcctggctcagctagcccgcgaggatgcggagcgggaactgaaggagaaggaggaggctcggaggaagaaggaactgctggagcagatggagaaggcccgccacgaacccatcaaccactcagatatggtggacaagatgtttggcttcctggggacttcaggcagcctgccaggccaggaaggccaggcgcctagtggctttgaggacctagagcgcggacggagggagatggtggaagaggatgttgacgctgccctgcccctgcctgatgaagacgaggaggacctttctgagtacaaattcgccaagtttgctgccacctacttccagggcacaaccacacactcctacacccggaggcctctcaagcagccgctgctctaccacgacgatgagggtgaccagctggcggcgctggctgtctggatcaccatcctccggttcatgggggacctcccagagcccaagtaccacacagccatgagcgacggcagtgagaagatcccagtgatgactaagatctacgagaccctaggcaagaagacatataagagggagctgcaggccttgcagggcgagggcgagacccagctccctgaggggcagaagaagaccagtgtgagacacaagttggtacacttgacactgaagaaaaagtccaaactcacagaagaggtgaccaagaggctgaacgatggggaatccacggtacagggcaacagcatgctggaggatcggcccacctcaaatctagagaagctgcacttcatcatcggcaacggcatcctgcggcctgcgctgcgggacgagatttactgccagatcagtaagcagctcacacacaacccatccaagagcagctatgccaggggctggatcctcgtgtcgctctgtgtgggctgcttcgccccctctgagaagttcgttaagtacctgcggaacttcatccacggaggcccacctggctatgctccttactgtgaggagcgcctgaggaggacctttgtcaacggaactcggacacagccacccagctggctggagctgcaggccaccaagtccaagaagcccatcatgttgcccgtgaccttcatggatgggaccaccaagaccctgctaacagattcagcaactacagccagggagctgtgcaatgctctggctgacaagatctcactcaaggaccgctttggcttctccctctacatcgctctgttcgataaggtgtcctccctgggcagcggcagtgaccatgtcatggatgccatctctcagtgtgagcagtacgccaaggagcagggtgctcaggagcgcaacgccccatggaggctcttctttagaaaggaggtcttcacaccctggcacaacccctcggaggacaacgtggccacgaacctcatctaccagcaggtggtgcgaggagtcaagtttggggagtacaggtgtgagaaggaggacgacctggctgagctggcttctcagcagtactttgtggactatggttctgagatgattctggagcgcctgctgagcctcgtgcccacttacatccctgaccgtgagatcacaccgctgaagaatcttgagaagtgggcacagctggccattgctgcccacaagaagggaatttatgcccagaggagaactgactcccagaaggtcaaagaggatgtggtcaattatgcccgtttcaagtggcccttgctcttctccaggttttacgaagcttacaaattctcaggccctcccctccccaagagcgacgtcatcgtggctgtcaactggacgggtgtgtacttcgtggacgagcaggagcaggtgcttctggagctgtccttcccggagatcatggctgtgtccagcagtaggggaacaaagatgatggcccccagctttaccctggccaccatcaaaggagatgagtacaccttcacatccagcaatgctgaggacatccgtgacctggtggtcacctttctggaggggctacggaagaggtctaagtatgtggtggcactgcaggacaatcctaaccctgctggtgaggagtcaggcttcctcagcttcgccaagggagacctcatcatccttgaccatgatactggtgagcaggtcatgaactcaggctgggccaacggcatcaacgagaggaccaagcagcgcggcgacttccccactgactgtgtatacgtcatgcccactgtcaccttgccaccaagggagattgtggccctggtcactatgaccccagaccagaggcaggatgtcgtccggctcctgcagcttcgcacagcagagccagaggtgcgcgccaagccctacacgctagaggagttctcctacgactacttcaggcccccacccaagcacacgctgagccgtgtcatggtgtccaaggcccgcggtaaggacaggctgtggagccacacacgagagcccctcaagcaggccctgctcaagaagatcctgggcagtgaagaactctcccaggaagcctgcatggcctttgtagctgtgctcaagtacatgggcgactacccatccaagaggatgcgatccgtcaatgagctcactgaccagatctttgagtgggcactcaaggctgagcccctcaaggatgaggcctacgtgcagatcctgaagcagctgactgacaatcacatcaggtacagcgaagagaggggctgggaactgctgtggctgtgcacgggcctcttcccgcccagcaacatcctcctgcctcatgttcagcggtttctgcagtcccgcaagcactgtcctcttgccattgactgcctgcagaggctccagaaagccctgagaaatggctcccggaagtaccctccgcacctggtggaggtggaggccatccaacataagactacccagatcttccacaaggtctacttccccgatgacacggacgaggcttttgaggtggagtccagcaccaaggccaaggacttctgccagaacatcgccagccggctgctgctcaagtcttccgagggattcagcctttttgtcaaaatcgcagataaggtcatcagcgtcccagagaatgatttcttctttgactttgtccgacacctgacagactggataaagaaagcacggcccatcaaggacggaatcgtgccctcactaacctaccaggtgttcttcatgaagaagctgtggaccaccacagtgccgggcaaggaccccatggctgactccatcttccactattaccaggaactgcccaaatatctccgaggctaccacaagtgcacccgggaggaggtgctgcagctgggcgcactcatctacagggtcaagtttgaggaggacaaatcctacttccctagcatccccaagttgctgagggagctggtaccccaggacctaatccggcaggtctcacctgatgactggaaacggtctattgtcgcctacttcaacaaacatgcggggaagtccaaggaggaagccaagctggccttcctcaaactcatcttcaagtggcccacctttggctcagccttctttgaggtgaagcaaactacagaaccaaacttcccagagattctcttaattgccatcaacaagtacggggtcagcctcatcgatcccagaaccaaggacatcctgactactcaccccttcaccaagatctccaactggagtagtggcaacacctacttccacatcaccattgggaacttggtccgtgggagcaaactgctctgtgagacatcgctgggatacaaaatggatgatcttctgacttcctacatcagccagatgctcacagccatgagcaagcagaggaactccaggagtggaaggtgaacctcagaggagacgctggctcaggccttggccctctaggcagggaagctggactgaccatatctgctgggcatctgatctgcctgccacgaggtccagactcttctgcatccacctatggcatctgggtttgctgtcaccctactttgttgtggccttcctggtgtaagagtctgtgttccttggtcacctctcctgattcagaccagctccatcaagcaacctcttttgactttctgtatatggatggcacagaggaatcaaggacaacttagctctctgcatacttggaacaaccaaactatttgtacattgaacggatgctctgaaacccaagggactgggctcagggtcctcagcactggcccctgtcataagcactaccactaaggactctctggaggactcctcagtatcatctgctccaggaagccccctagactacctcctgagtctggacaaagcctcctgattctacctggatcactcctgttatgtgacagttatgtggtgggtccctgctaaaatctccctgaccacctgagggcataaagcatgtgtcttattctctgg

SEQ ID NO: 12 - protein, 2166 aa, Mus musculus
MDLKSGQEFDVPIGAVVKLCDSGQIQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNNRDQCCIISGESGAGKTESTKLILQFLAAISGQHSWIEQQVLEATPILEAFGNAKTIRNDNSSRFGKYIDIHFNKRGAIEGAKIEQYLLEKSRVCRQAPDERNYHVFYCMLEGMNEEEKKKLGLGQAADYNYLAMGNCITCEGRVDSQEYANIRSAMKVLMFTDTENWEISKLLAAILHMGNLQYEARTFENLDACEVLFSPSLATAASLLEVNPPDLMSCLTSRTLITRGETVSTPLSREQALDVRDAFVKGIYGRLFVWIVEKINAAIYKPPPLEVKNSRRSIGLLDIFGFENFTVNSFEQLCINFANEHLQQFFVRHVFKLEQEEYDLESIDWLHIEFTDNQEALDMIANRPMNVISLIDEESKFPKGTDATMLHKLNSQHKLNANYVPPKNSHETQFGINHFAGVVYYESQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRHAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLRLKSAATLIQRHWRGHHCRKNYELIRLGFLRLQALHRSRKLHKQYRLARQRIIEFQARCRAYLVRKAFRHRLWAVITVQAYARGMIARRLHRRLRVEYQRRLEAERMRLAEEEKLRKEMSAKKAKEEAERKHQERLAQLAREDAERELKEKEEARRKKELLEQMEKARHEPINHSDMVDKMFGFLGTSGSLPGQEGQAPSGFEDLERGRREMVEEDVDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGETQLPEGQKKTSVRHKLVHLTLKKKSKLTEEVTKRLNDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTARELCNALADKISLKDRFGFSLYIALFDKVSSLGSGSDHVMDAISQCEQYAKEQGAQERNAPWRLFFRKEVFTPWHNPSEDNVATNLIYQQVVRGVKFGEYRCEKEDDLAELASQQYFVDYGSEMILERLLSLVPTYIPDREITPLKNLEKWAQLAIAAHKKGIYAQRRTDSQKVKEDVVNYARFKWPLLFSRFYEAYKFSGPPLPKSDVIVAVNWTGVYFVDEQEQVLLELSFPEIMAVSSSRGTKMMAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDCVYVMPTVTLPPREIVALVTMTPDQRQDVVRLLQLRTAEPEVRAKPYTLEEFSYDYFRPPPKHTLSRVMVSKARGKDRLWSHTREPLKQALLKKILGSEELSQEACMAFVAVLKYMGDYPSKRMRSVNELTDQIFEWALKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIASRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPRTKDILTTHPFTKISNWSSGNTYFHITIGNLVRGSKLLCETSLGYKMDDLLTSYISQMLTAMSKQRNSRSGR

SEQ ID NO: 13 - DNA, 7361 bp, Mus musculus
agtgcaggctggacagctgccctgaacagaaagaaagagtgacccagggagacaagaaacagagtagcccaagggaagcccacagcagcagcagatcaaggctcaagctggagctgaaaatttgcaggctccagcctcagcttccagagtcctcctgacctgtgacccctggctcctggctgggaggtggtgactcggagggtgtggataaaacccagagctgtgtctggtcactccggcaggtgtgctgacgtagaagcatggttattctgcagaagggggactatgtatggatggacctgaagtcaggccaggagtttgatgtgcccatcggggccgtggtgaagctctgcgactcgggccagatccaggtggtggatgatgaagacaatgaacactggatatcccctcagaatgccacgcacatcaagccaatgcaccccacatcggtgcacggcgtggaggacatgatccgcctgggggatctcaacgaggcaggcatccttcgaaaccttctcattcgctaccgggaccacctcatctatacgtacacaggttccatcctggtggccgtgaacccctaccagctgctctccatctactcgccagagcacatccgccagtacaccaacaagaagataggggagatgcccccccacatcttcgccattgctgacaactgctacttcaacatgaaacgcaacaaccgggaccagtgctgtattatcagcggggagtcgggagctggcaagacagagagcacaaagttgatcctgcagttcctggcagccatcagtggacagcactcatggatcgagcagcaggtgctggaggccaccccgatcctggaagcatttgggaacgccaagaccatccgcaacgacaactctagccgctttggcaagtacattgacatccactttaacaagcgtggtgccatcgagggcgccaaaatagagcaatacctgctggagaagtcacgtgtctgccgccaggcccctgacgagaggaactatcacgtgttctactgtatgctggagggcatgaatgaggaggagaagaagaaactgggcctaggccaggccgctgactacaactacttggccatgggtaactgcatcacctgtgagggccgcgtggacagtcaggagtatgccaacatccgctctgccatgaaggttctcatgttcacagacacggagaactgggagatctcgaagcttctggctgccatcctacacatgggcaatctgcagtatgaggcccggacatttgagaacttggatgcgtgtgaagtcctcttctccccatcgctggccacggcagcttctctgctcgaggtgaaccccccagacctgatgagctgcctcaccagccgcaccctcatcacccgtggggagacggtgtccacccctctcagcagggaacaggcgctggatgtgcgagatgcctttgtcaagggcatctatgggcggctctttgtgtggattgtggagaagatcaacgcagcaatctacaagccaccccccctggaagtgaagaactctcgccggtccatcggtctcctggacatctttggatttgagaacttcactgtgaacagcttcgagcagctctgcattaactttgccaatgagcacctgcagcaattcttcgtgcggcacgtgttcaagctggagcaggaggagtacgacctggagagcatcgactggttgcacattgagttcactgacaaccaggaagcactggacatgattgccaaccggcctatgaacgtcatctccctcatcgatgaggagagcaagttccccaagggcacggatgccaccatgctgcataagctgaactcacagcacaagctcaatgccaactacgtgccacccaagaacagccacgagacccagtttggaatcaaccactttgcgggtgttgtctattatgagagtcaaggcttcctggagaagaaccgagacaccctgcatggggacatcatccagctggtccactcttcccggaacaagttcataaagcagattttccaagctgacgttgccatgggtgccgagaccaggaagcgctcgcctacactcagcagccagttcaagcggtctctggagctgctgatgcgcacactgggcgcctgccagcccttctttgtgcgttgtatcaaacccaatgagttcaagaagcccatgctcttcgaccggcacttgtgtgtacgccagctgcgatattcgggcatgatggagacaatccgcatccgccacgcaggctaccccattcgctacagctttgtggagtttgtggagcgctaccgggtactgctgcctggtgtgaagccagcatacaagcagggtgacctccgagggacatgccagcgcatggctgaggctgtgctgggcacgcacgatgactggcagattggcaaaaccaagatctttctgaaggaccaccatgacatgttgctggaggtggagcgggacaaggccatcacagacagagtcattctcctccagaaggttatccggggcttcaaagacaggtccaacttcctgagactgaagagtgctgccacactgatccagaggcactggcggggccaccactgtaggaaaaactatgagctgattcgtcttggcttcctgcggctgcaggccctgcaccgctcccggaagctgcacaagcagtaccgcctggccagacagcgcataatagagttccaggcccgctgccgggcctatctggtgcgcaaggccttccgccaccgcctctgggccgtgatcaccgtgcaggcctatgcccgaggcatgattgcccgccggctacaccgccgcctccgggttgagtaccagcggcgcctcgaggcagagaggatgcgtctggcagaggaggagaaactccgaaaggagatgagtgccaagaaggccaaagaggaggctgagcgcaagcatcaggagcgcctggctcagctagcccgcgaggatgcggagcgggaactgaaggagaaggaggaggctcggaggaagaaggaactgctggagcagatggagaaggcccgccacgaacccatcaaccactcagatatggtggacaagatgtttggcttcctggggacttcaggcagcctgccaggccaggaaggccaggcgcctagtggctttgaggacctagagcgcggacggagggagatggtggaagaggatgttgacgctgccctgcccctgcctgatgaagacgaggaggacctttctgagtacaaattcgccaagtttgctgccacctacttccagggcacaaccacacactcctacacccggaggcctctcaagcagccgctgctctaccacgacgatgagggtgaccagctggcggcgctggctgtctggatcaccatcctccggttcatgggggacctcccagagcccaagtaccacacagccatgagcgacggcagtgagaagatcccagtgatgactaagatctacgagaccctaggcaagaagacatataagagggagctgcaggccttgcagggcgagggcgagacccagctccctgaggggcagaagaagaccagtgtgagacacaagttggtacacttgacactgaagaaaaagtccaaactcacagaagaggtgaccaagaggctgaacgatggggaatccacggtacagggcaacagcatgctggaggatcggcccacctcaaatctagagaagctgcacttcatcatcggcaacggcatcctgcggcctgcgctgcgggacgagatttactgccagatcagtaagcagctcacacacaacccatccaagagcagctatgccaggggctggatcctcgtgtcgctctgtgtgggctgcttcgccccctctgagaagttcgttaagtacctgcggaacttcatccacggaggcccacctggctatgctccttactgtgaggagcgcctgaggaggacctttgtcaacggaactcggacacagccacccagctggctggagctgcaggccaccaagtccaagaagcccatcatgttgcccgtgaccttcatggatgggaccaccaagaccctgctaacagattcagcaactacagccagggagctgtgcaatgctctggctgacaagatctcactcaaggaccgctttggcttctccctctacatcgctctgttcgataaggtgtcctccctgggcagcggcagtgaccatgtcatggatgccatctctcagtgtgagcagtacgccaaggagcagggtgctcaggagcgcaacgccccatggaggctcttctttagaaaggaggtcttcacaccctggcacaacccctcggaggacaacgtggccacgaacctcatctaccagcaggtggtgcgaggagtcaagtttggggagtacaggtgtgagaaggaggacgacctggctgagctggcttctcagcagtactttgtggactatggttctgagatgattctggagcgcctgctgagcctcgtgcccacttacatccctgaccgtgagatcacaccgctgaagaatcttgagaagtgggcacagctggccattgctgcccacaagaagggaatttatgcccagaggagaactgactcccagaaggtcaaagaggatgtggtcaattatgcccgtttcaagtggcccttgctcttctccaggttttacgaagcttacaaattctcaggccctcccctccccaagagcgacgtcatcgtggctgtcaactggacgggtgtgtacttcgtggacgagcaggagcaggtgcttctggagctgtccttcccggagatcatggctgtgtccagcagtaggggaacaaagatgatggcccccagctttaccctggccaccatcaaaggagatgagtacaccttcacatccagcaatgctgaggacatccgtgacctggtggtcacctttctggaggggctacggaagaggtctaagtatgtggtggcactgcaggacaatcctaaccctgctggtgaggagtcaggcttcctcagcttcgccaagggagacctcatcatccttgaccatgatactggtgagcaggtcatgaactcaggctgggccaacggcatcaacgagaggaccaagcagcgcggcgacttccccactgactgtgtatacgtcatgcccactgtcaccttgccaccaagggagattgtggccctggtcactatgaccccagaccagaggcaggatgtcgtccggctcctgcagcttcgcacagcagagccagaggtgcgcgccaagccctacacgctagaggagttctcctacgactacttcaggcccccacccaagcacacgctgagccgtgtcatggtgtccaaggcccgcggtaaggacaggctgtggagccacacacgagagcccctcaagcaggccctgctcaagaagatcctgggcagtgaagaactctcccaggaagcctgcatggcctttgtagctgtgctcaagtacatgggcgactacccatccaagaggatgcgatccgtcaatgagctcactgaccagatctttgagtgggcactcaaggctgagcccctcaaggatgaggcctacgtgcagatcctgaagcagctgactgacaatcacatcaggtacagcgaagagaggggctgggaactgctgtggctgtgcacgggcctcttcccgcccagcaacatcctcctgcctcatgttcagcggtttctgcagtcccgcaagcactgtcctcttgccattgactgcctgcagaggctccagaaagccctgagaaatggctcccggaagtaccctccgcacctggtggaggtggaggccatccaacataagactacccagatcttccacaaggtctacttccccgatgacacggacgaggcttttgaggtggagtccagcaccaaggccaaggacttctgccagaacatcgccagccggctgctgctcaagtcttccgagggattcagcctttttgtcaaaatcgcagataaggtcatcagcgtcccagagaatgatttcttctttgactttgtccgacacctgacagactggataaagaaagcacggcccatcaaggacggaatcgtgccctcactaacctaccaggtgttcttcatgaagaagctgtggaccaccacagtgccgggcaaggaccccatggctgactccatcttccactattaccaggaactgcccaaatatctccgaggctaccacaagtgcacccgggaggaggtgctgcagctgggcgcactcatctacagggtcaagtttgaggaggacaaatcctacttccctagcatccccaagttgctgagggagctggtaccccaggacctaatccggcaggtctcacctgatgactggaaacggtctattgtcgcctacttcaacaaacatgcggggaagtccaaggaggaagccaagctggccttcctcaaactcatcttcaagtggcccacctttggctcagccttctttgaggtgaagcaaactacagaaccaaacttcccagagattctcttaattgccatcaacaagtacggggtcagcctcatcgatcccagaaccaaggacatcctgactactcaccccttcaccaagatctccaactggagtagtggcaacacctacttccacatcaccattgggaacttggtccgtgggagcaaactgctctgtgagacatcgctgggatacaaaatggatgatcttctgacttcctacatcagccagatgctcacagccatgagcaagcagaggaactccaggagtggaaggtgaacctcagaggagacgctggctcaggccttggccctctaggcagggaagctggactgaccatatctgctgggcatctgatctgcctgccacgaggtccagactcttctgcatccacctatggcatctgggtttgctgtcaccctactttgttgtggccttcctggtgtaagagtctgtgttccttggtcacctctcctgattcagaccagctccatcaagcaacctcttttgactttctgtatatggatggcacagaggaatcaaggacaacttagctctctgcatacttggaacaaccaaactatttgtacattgaacggatgctctgaaacccaagggactgggctcagggtcctcagcactggcccctgtcataagcactaccactaaggactctctggaggactcctcagtatcatctgctccaggaagccccctagactacctcctgagtctggacaaagcctcctgattctacctggatcactcctgttatgtgacagttatgtggtgggtccctgctaaaatctccctgaccacctgagggcataaagcatgtgtcttatt

SEQ ID NO: 14 - protein, 2177 aa, Mus musculus
MVILQKGDYVWMDLKSGQEFDVPIGAVVKLCDSGQIQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNNRDQCCIISGESGAGKTESTKLILQFLAAISGQHSWIEQQVLEATPILEAFGNAKTIRNDNSSRFGKYIDIHFNKRGAIEGAKIEQYLLEKSRVCRQAPDERNYHVFYCMLEGMNEEEKKKLGLGQAADYNYLAMGNCITCEGRVDSQEYANIRSAMKVLMFTDTENWEISKLLAAILHMGNLQYEARTFENLDACEVLFSPSLATAASLLEVNPPDLMSCLTSRTLITRGETVSTPLSREQALDVRDAFVKGIYGRLFVWIVEKINAAIYKPPPLEVKNSRRSIGLLDIFGFENFTVNSFEQLCINFANEHLQQFFVRHVFKLEQEEYDLESIDWLHIEFTDNQEALDMIANRPMNVISLIDEESKFPKGTDATMLHKLNSQHKLNANYVPPKNSHETQFGINHFAGVVYYESQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRHAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLRLKSAATLIQRHWRGHHCRKNYELIRLGFLRLQALHRSRKLHKQYRLARQRIIEFQARCRAYLVRKAFRHRLWAVITVQAYARGMIARRLHRRLRVEYQRRLEAERMRLAEEEKLRKEMSAKKAKEEAERKHQERLAQLAREDAERELKEKEEARRKKELLEQMEKARHEPINHSDMVDKMFGFLGTSGSLPGQEGQAPSGFEDLERGRREMVEEDVDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGETQLPEGQKKTSVRHKLVHLTLKKKSKLTEEVTKRLNDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTARELCNALADKISLKDRFGFSLYIALFDKVSSLGSGSDHVMDAISQCEQYAKEQGAQERNAPWRLFFRKEVFTPWHNPSEDNVATNLIYQQVVRGVKFGEYRCEKEDDLAELASQQYFVDYGSEMILERLLSLVPTYIPDREITPLKNLEKWAQLAIAAHKKGIYAQRRTDSQKVKEDVVNYARFKWPLLFSRFYEAYKFSGPPLPKSDVIVAVNWTGVYFVDEQEQVLLELSFPEIMAVSSSRGTKMMAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDCVYVMPTVTLPPREIVALVTMTPDQRQDVVRLLQLRTAEPEVRAKPYTLEEFSYDYFRPPPKHTLSRVMVSKARGKDRLWSHTREPLKQALLKKILGSEELSQEACMAFVAVLKYMGDYPSKRMRSVNELTDQIFEWALKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIASRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPRTKDILTTHPFTKISNWSSGNTYFHITIGNLVRGSKLLCETSLGYKMDDLLTSYISQMLTAMSKQRNSRSGR

SEQ ID NO: 15 - DNA, 881 bp, Artificial Sequence
  Synthetic
ctgagggaacagagtggctattaccaagccactgccttccaggacctccttacccacgtcctcccaacctcccctgactttctcagtagctaccgccatcacaccccgacacaggctcttggcctcactgccaatctgtgaggtgggtgaaccacataaccttagatgcacattccaggctagggcatctgtttctcatgactaaaatgattagaaagctagcgctggaggacgtctagagcccaggagttcaaggctagcctgatcaatagagcaagactctctaatctcaccgtttccccctgagagcaatactatgtttccttccccaggtcaagccaattctatcattagcatctatcctgaagcagctgtgtaaatcttgatggtttggcccagggccagtgggaagcagacaatccctgctccccatgtgaaccccctagaatcagtgcagagcaccagaacaggctcatgcggcttctcccaggttgcagatgctggggggagctgaggcttgctgtgcccaccttggggaacttgctctcctcatcgatgagggagatgacgttcataggcgggttggcaatcatgtccagtgcttcctggttgtcagtgaactcaatgtgcaaccagtcgatgctctccaggtcgtactcctcctgctccagcttgaacacgtgccgcacgaagaattgctgcaggtgctcattggcaaagttaatgcagagctgctcgaagctgcagagggaagaggaccttggacatgtggcgcccaacttctgccctgtcacccagacccggctctacctagatcacagcttgacacaagactcccatcacgtgggctagggtacaactatcaagtccaactcctacgc

SEQ ID NO: 16 - DNA, 23 bp, Artificial Sequence
  Synthetic
gatgacgttcataggccggttgg

SEQ ID NO: 17 - DNA, 23 bp, Artificial Sequence
  Synthetic
cttgctctcctcatcgatgaggg

SEQ ID NO: 18 - DNA, 23 bp, Artificial Sequence
  Synthetic
atgagggagatgacgttcatagg

SEQ ID NO: 19 - DNA, 23 bp, Artificial Sequence
  Synthetic
agggagatgacgttcataggcgg

SEQ ID NO: 20 - DNA, 23 bp, Artificial Sequence
  Synthetic
caatcatgtccagtgcttcctgg

SEQ ID NO: 21 - DNA, 23 bp, Artificial Sequence
  Synthetic
ccaaccggcctatgaacgtcatc

SEQ ID NO: 22 - DNA, 23 bp, Artificial Sequence
  Synthetic
ccctcatcgatgaggagagcaag

SEQ ID NO: 23 - DNA, 23 bp, Artificial Sequence
  Synthetic
cctatgaacgtcatctccctcat

SEQ ID NO: 24 - DNA, 23 bp, Artificial Sequence
  Synthetic
ccgcctatgaacgtcatctccct

SEQ ID NO: 25 - DNA, 23 bp, Artificial Sequence
  Synthetic
ccaggaagcactggacatgattg

SEQ ID NO: 26 - DNA, 61 bp, Artificial Sequence
  Synthetic
tcatcgatgagggagatgacgttcataggcgggttggcaatcatgtccagtgcttcctggt

SEQ ID NO: 27 - DNA, 61 bp, Artificial Sequence
  Synthetic
accaggaagcactggacatgattgccaacccgcctatgaacgtcatctccctcatcgatga

SEQ ID NO: 28 - DNA, 72 bp, Artificial Sequence
  Synthetic
aaccaggaagcactggacatgattgccaaccggcctatgaacgtcatctccctcatcgatgaggagagcaag

SEQ ID NO: 29 - DNA, 72 bp, Artificial Sequence
  Synthetic
aatcaagaagcactggacatgattgccaatcggccvatgaacgtcatctcdctcatcgatgaggagagcaag

SEQ ID NO: 30 - DNA, 96 bp, Artificial Sequence
  Synthetic
acaaccaggaagcactggacatgattgccaaccggcctatgaacgtcatctcactcattgatgaagagagcaagttccccaaggtgggcacagcaa

SEQ ID NO: 31 - DNA, 22 bp, Artificial Sequence
  Synthetic
gagggaacagagtggctattac

SEQ ID NO: 32 - DNA, 22 bp, Artificial Sequence
  Synthetic
gcgtaggagttggacttgatag

SEQ ID NO: 33 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 34-39 - skipped (6 entries)

SEQ ID NO: 40 - RNA, 20 nt, Artificial Sequence
  Synthetic
gaugacguucauaggcgggu

SEQ ID NO: 41 - RNA, 17 nt, Artificial Sequence
  Synthetic
gacguucauaggcgggu

SEQ ID NO: 42 - RNA, 20 nt, Artificial Sequence
  Synthetic
agggagaugacguucauagg

SEQ ID NO: 43 - RNA, 17 nt, Artificial Sequence
  Synthetic
gagaugacguucauagg

SEQ ID NO: 44 - RNA, 20 nt, Artificial Sequence
  Synthetic
cuugcucuccucaucgauga

SEQ ID NO: 45 - RNA, 20 nt, Artificial Sequence
  Synthetic
augagggagaugacguucau

SEQ ID NO: 46 - DNA, 20 bp, Artificial Sequence
  Synthetic
acccgcctatgaacgtcatc

SEQ ID NO: 47 - DNA, 17 bp, Artificial Sequence
  Synthetic
acccgcctatgaacgtc

SEQ ID NO: 48 - DNA, 20 bp, Artificial Sequence
  Synthetic
cctatgaacgtcatctccct

SEQ ID NO: 49 - DNA, 17 bp, Artificial Sequence
  Synthetic
cctatgaacgtcatctc

SEQ ID NO: 50 - DNA, 20 bp, Artificial Sequence
  Synthetic
tcatcgatgaggagagcaag

SEQ ID NO: 51 - DNA, 96 bp, Artificial Sequence
  Synthetic
ttgctgtgcccaccttggggaacttgctctcttcatcaatgagtgagatgacgttcataggccggttggcaatcatgtccagtgcttcctggttgt

SEQ ID NO: 52 - DNA, 54 bp, Artificial Sequence
  Synthetic
acactctttccctacacgacgctcttccgatctcccaggtcaagccaattctat

SEQ ID NO: 53 - DNA, 52 bp, Artificial Sequence
  Synthetic
gactggagttcagacgtgtgctcttccgatctcttcgagcagctctgcatta

SEQ ID NO: 54 - DNA, 74 bp, Artificial Sequence
  Synthetic
aaccaggaagcactggacatgattgccaacccgcctatgaacgtcatctccctcatcgatgaggagagcaagtt

SEQ ID NO: 55 - DNA, 74 bp, Artificial Sequence
  Synthetic
aacttgctctcctcatcgatgagggagatgacgttcataggcgggttggcaatcatgtccagtgcttcctggtt

SEQ ID NO: 56 - DNA, 23 bp, Artificial Sequence
  Synthetic
caatcatgtcaagtgcttcctgg

SEQ ID NO: 57 - DNA, 74 bp, Artificial Sequence
  Synthetic
aaccaggaagcactggacatgattgccaaccggcctatgaacgtcatctccctcatcgatgaggagagcaagtt

SEQ ID NO: 58 - DNA, 74 bp, Artificial Sequence
  Synthetic
aacttgctctcctcatcgatgagggagatgacgttcataggccggttggcaatcatgtccagtgcttcctggtt

SEQ ID NO: 59 - DNA, 72 bp, Artificial Sequence
  Synthetic
  misc_feature (36)..(36): n is a, c, g, or t
  misc_feature (51)..(51): n is a, c, g, or t
aaycargaagcactggacatgattgccaaycggccnatgaacgtcatctcnctcatcgatgaggagagcaag

SEQ ID NO: 60 - DNA, 72 bp, Artificial Sequence
  Synthetic
ccaggaagcactggacatgattgccaaccggcctatgaacgtcatctccctcatcgatgaggagagcaagtt

SEQ ID NO: 61 - DNA, 71 bp, Artificial Sequence
  Synthetic
ccaggaagcactggacatgattgccaacccgcctatgaacgtcactccctcatcgatgaggagagcaagtt

SEQ ID NO: 62 - DNA, 74 bp, Artificial Sequence
  Synthetic
aaccacgaagcactggacatgattgccaacccgcctatgaacgtcatctccctcatcgatgaggagagcaagtt

SEQ ID NO: 63 - protein, 24 aa, Artificial Sequence
  Synthetic
NQEALDMIANPPMNVISLIDEESK

SEQ ID NO: 64 - DNA, 24 bp, Artificial Sequence
  Synthetic
cgttcataggcsggttggcaatca

(19) United States
(12) Patent Application Publication
(10) Pub. No.: US 2024/0189451 A1
(43) Pub. Date: Jun. 13, 2024

(54) EXOSOME GENE THERAPY FOR TREATING INNER EAR DISEASE

(71) Applicant: University of Florida Research Foundation, Incorporated, Gainesville, FL (US)

(72) Inventors: Mei He, Gainesville, FL (US); Xiaoshu Pan, Gainesville, FL (US)

(73) Assignee: University of Florida Research Foundation, Incorporated, Grainesville, FL (US)

(21) Appl. No.: 18/286,450

(22) PCT Filed: Mar. 31, 2022
(86) PCT No.: PCT/US2022/022832

§ 371 Date: Oct. 11, 2023

(51) Int. Cl. A61K 48/00; A61K 9/50; A61K 47/26; A61P 27/16; C12N 9/22; C12N 15/11; C12N 15/88

(52) U.S. Cl. CPC A61K 48/005; A61K 9/5063; A61K 47/26; A61P 27/16; C12N 9/22; C12N 15/111; C12N 15/88; C12N 2310/20

(57) ABSTRACT

Provided herein are compositions and methods useful in the treatment of hearing loss diseases, such as by correction of mutations in genes associated with hearing.

### RELATED APPLICATIONS

This application claims the benefit under 35 U.S.C. § 119(e) of U.S. Provisional Application No. 63/173,662, entitled “EXOSOME GENE THERAPY FOR TREATING INNER EAR DISEASE”, filed on Apr. 12, 2021, the contents of which are incorporated herein by reference in their entirety.

### GOVERNMENT SUPPORT

This invention was made with government support under R35 GM133794 awarded by the National Institutes of Health. The government has certain rights in the invention.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Mar. 31, 2022, is named U119770191WO00-SEQ-COB and is 212,873 bytes in size.

### BACKGROUND

Sensorineural hearing loss (SNHL) is one of the most common neurodegenerative diseases and contributes to nearly 90% of all hearing loss disease. Among hearing loss diseases, 50-60% have genetic causes based on homozygous recessive mutations that induce severe hereditary hearing loss within family trees. The deafness resulting from genotype to phenotype expression has been well-defined, resulting in a foundation for developing gene replacement therapies via exogenous expression of wild-type genes. However, no efficient and targeted delivery approaches are available for facilitating such transgene expression in vivo. Existing delivery approaches for SNHL include intratympanic injection and hydrogel delivery of drugs into the ear, each of which exhibit poor penetration of therapeutics through the blood-labyrinth barrier to the inner ear.

### SUMMARY

The present disclosure is based in part on the development of CRISPR/Cas endonuclease (e.g., Cas9) compositions and methods for providing functional genes to cells harboring SNHL-associated mutations. As such, some aspects of the present disclosure relate to CRISPR/CRISPR-associated endonuclease (Cas endonuclease, e.g., Cas9) compositions, including guide RNAs and template nucleic acids, as well as methods of their use.

Extracellular vesicles, such as exosomes, prepared according to methods described herein have the useful advantage of overcoming the challenges of therapeutic delivery to the inner ear. As such, some aspects of the present disclosure relate to methods of preparing extracellular vesicles, such as to include CRISPR/Cas endonuclease (e.g., Cas9) compositions disclosed herein.

According to some aspects, methods related to gene editing are provided herein. In some embodiments, a method comprises providing to a subject a CRISPR-associated endonuclease, a guide RNA (gRNA), and a template nucleic acid, wherein the gRNA targets a MYO7A gene.

In some embodiments, the CRISPR-associated endonuclease is Cas9. In some embodiments, the CRISPR-associated endonuclease is provided as a protein. In some embodiments, the CRISPR-associated endonuclease is provided as a nucleic acid encoding a protein. In some embodiments, the nucleic acid is a messenger RNA (mRNA). In some embodiments, the CRISPR-associated endonuclease and the gRNA are provided as a ribonucleoprotein (RNP) complex or a nucleic acid encoding an RNP complex.

In some embodiments, the template nucleic acid comprises a portion of a nucleic acid sequence encoding a wild-type MYO7A protein or a sequence capable of specifically binding to a portion of a nucleic acid sequence encoding a wild-type MYO7A protein. In some embodiments, the wild-type MYO7A protein is a mammalian MYO7A protein. In some embodiments, the wild-type MYO7A protein is a human MYO7A protein. In some embodiments, the wild-type MYO7A protein is a mouse MYO7A protein.

In some embodiments, the gRNA comprises, consists essentially of, or consists of a nucleic acid sequence of 10-30 or 15-25 consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13), or a nucleotide sequence of 10-30 or 15-25 nucleotides capable of specifically hybridizing to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises, consists essentially of, or consists of a nucleic acid sequence of, or capable of specifically binding to any one of the sequences of

(SEQ ID NO: 16)
GATGACGTTCATAGGCCGGTTGG,
(SEQ ID NO: 17)
CTTGCTCTCCTCATCGATGAGGG,
(SEQ ID NO: 18)
ATGAGGGAGATGACGTTCATAGG,
(SEQ ID NO: 19)
AGGGAGATGACGTTCATAGGCGG,
(SEQ ID NO: 20)
CAATCATGTCCAGTGCTTCCTGG,
(SEQ ID NO: 40)
GAUGACGUUCAUAGGCGGGU,
(SEQ ID NO: 41)
GACGUUCAUAGGCGGGU,
(SEQ ID NO: 42)
AGGGAGAUGACGUUCAUAGG,
(SEQ ID NO: 43)
GAGAUGACGUUCAUAGG,
(SEQ ID NO: 44)
CUUGCUCUCCUCAUCGAUGA,
or
(SEQ ID NO: 45)
AUGAGGGAGAUGACGUUCAU,

wherein each uracil base (U) may independently and optionally be replaced with a thymine base (T) and each T may independently and optionally be replaced with a U.

In some embodiments, the gRNA comprises, consists essentially of, or consists of a nucleotide sequence of 10-30 or 15-25 consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5) or a nucleotide sequence of 10-30 or 15-25 nucleotides capable of specifically hybridizing to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5).

In some embodiments, the MYO7A gene is a mouse MYO7A gene. In some embodiments, the MYO7A gene is a human MYO7A gene.

In some embodiments, the CRISPR-associated endonuclease, the gRNA, and/or the template nucleic acid are encapsulated within an extracellular vesicle. In some embodiments, the extracellular vesicle is an exosome.

According to some aspects, compositions related to gene editing are provided herein. In some embodiments, a composition comprises a CRISPR-associated endonuclease or a nucleic acid sequence encoding a CRISPR-associated endonuclease, a guide RNA (gRNA), and a template nucleic acid, wherein the gRNA is targets a MYO7A gene.

In some embodiments, the composition is comprised within an extracellular vesicle. In some embodiments, the extracellular vesicle is an exosome.

In some embodiments, the composition further comprises a stabilizing agent. In some embodiments, the stabilizing agent is a disaccharide. In some embodiments, the stabilizing agent is trehalose. In some embodiments, the stabilizing agent is associated with the extracellular vesicle.

In some embodiments, the CRISPR-associated endonuclease is Cas9.

In some embodiments, the composition comprises a CRISPR-associated endonuclease. In some embodiments, the composition comprises a nucleic acid encoding a CRISPR-associated endonuclease.

In some embodiments, the template nucleic acid comprises a portion of a nucleic acid sequence encoding a wild-type MYO7A protein.

In some embodiments, the gRNA comprises, consists essentially of, or consists of a nucleic acid sequence of 10-30 or 15-25 consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13), or a nucleotide sequence of 10-30 or 15-25 nucleotides capable of specifically hybridizing to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises, consists essentially of, or consists of a nucleic acid sequence of, or capable of specifically binding to any one of the sequences of

(SEQ ID NO: 16)
GATGACGTTCATAGGCCGGTTGG,
(SEQ ID NO: 17)
CTTGCTCTCCTCATCGATGAGGG,
(SEQ ID NO: 18)
ATGAGGGAGATGACGTTCATAGG,
(SEQ ID NO: 19)
AGGGAGATGACGTTCATAGGCGG,
(SEQ ID NO: 20)
CAATCATGTCCAGTGCTTCCTGG,
(SEQ ID NO: 40)
GAUGACGUUCAUAGGCGGGU,
(SEQ ID NO: 41)
GACGUUCAUAGGCGGGU,
(SEQ ID NO: 42)
AGGGAGAUGACGUUCAUAGG,
(SEQ ID NO: 43)
GAGAUGACGUUCAUAGG,
(SEQ ID NO: 44)
CUUGCUCUCCUCAUCGAUGA,
or
(SEQ ID NO: 45)
AUGAGGGAGAUGACGUUCAU,

wherein each uracil base (U) may independently and optionally be replaced with a thymine base (T) and each T may independently and optionally be replaced with a U.

In some embodiments, the gRNA comprises, consists essentially of, or consists of a nucleotide sequence of 10-30 or 15-25 consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5) or a nucleotide sequence of 10-30 or 15-25 nucleotides capable of specifically hybridizing to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5).

In some embodiments, the MYO7A gene is a mouse MYO7A gene. In some embodiments, the MYO7A gene is a human MYO7A gene.

According to some aspects, methods of treating a hearing disorder are provided herein. In some embodiments, a method of treating a hearing loss disorder comprises administering to a subject in need thereof a composition disclosed herein in an amount sufficient to treat a hearing loss disorder in the subject. In some embodiments, the subject is a mammal. In some embodiments, the subject is a primate. In some embodiments, the subject is a human.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1F show details of exosome-mediated delivery of cargoes. FIG. 1A shows a schematic illustration of inner ear structure and the blood labyrinth barrier (BLB). FIG. 1B shows an optical microscopy image of the morphology of HEI-OC1 cells in culture (top) and stained for myosin VIIa/MYO7A protein in the cytoplasm (bottom). FIG. 1C shows scanning electron microscopy (SEM) images of exosomes before electro-transfection (top) or trehalose-treated exosomes after electro-transfection (bottom), showing maintenance of the stable and round vesicle morphology following electro-transfection in trehalose-treated exosomes. FIG. 1D shows nanoparticle tracking analysis (NTA) of exosomes before and after electro-transfection, demonstrating a stable size distribution around approximately 150 nm. FIG. 1E shows proof-of-concept measurements of transfection (bars) and gene expression (circles) by exosomes treated with various concentrations of trehalose during electro-transfection. FIG. 1F shows quantification of cell viability using 3-(4,5-dimethylthiazol-2-yl)-2,5-diphenyltetrazolium bromide (MTT) assay following treatment of cells with electro-transfected exosomes in vitro, compared with untreated control cells, demonstrating low toxicity and good biocompatibility of electro-transfected exosomes.

FIG. 2 shows a schematic illustration of exosome-mediated gene editing of MYO7A in hair cells. ODN: oligodeoxynucleotide donor template; HDR: homology-directed repair.

FIG. 3 shows a schematic illustration of a missense mutation in Myo7A highlighting the G1601C mutation, which results in an arginine (R) to proline (P) substitution. The “positive control” guide RNA (gRNA; described in Example 1 and the related Figures herein as “gRNA5”) labeled 1 is commercially available and is not able to facilitate editing of Myo7A in vitro. The remaining gRNAs (labeled “self-designed”; described in Example 1 and the related Figures herein as “gRNA1”, “gRNA2”, “gRNA3”, and “gRNA4”, respectively) were designed to facilitate editing. The scissors indicate the cutting site within MYO7A for each gRNA. The amino acids in rectangles show the R502P mutation and flanking amino acids. Silent mutations in the single-stranded oligodeoxynucleotide donor template (ssODN) are boxed in bold. In the ssODN, V represents A, C, or G; D represents A, G, or T. Sequences shown correspond (top-bottom) to SEQ ID NOs: 54, 55, 56, 16, 17, 18, 19, 57, 58, and 59.

FIG. 4 shows an electrophoresis gel of MYO7A^{sh1} amplicons tested in a cell-free Cas9 cutting assay. Lanes 1 and 8 show size ladders. Lane 2 shows an untreated MYO7A^{sh1} amplicon amplified from murine car fibroblast cell genomic DNA. Lanes 3-7 show MYO7A^{sh1} amplicons treated with Cas9 protein and gRNA1, gRNA2, gRNA3, gRNA4, and gRNA5, respectively. These results demonstrate Cas9/gRNA can facilitate cleavage of MYO7A^{sh1} DNA. The primers used to amplify the MYO7A^{sh1} amplicons in this figure were

forward
(SEQ ID NO: 31)
5′-GAGGGAACAGAGTGGCTATTAC-3′
and
reverse
(SEQ ID NO: 32)
5′-GCGTAGGAGTTGGACTTGATAG-3′.

FIG. 5 shows an electrophoresis gel of MYO7A^{sh1} amplicons tested in a cell-free cleavage assay using EGFP tagged ribonucleoprotein (RNP) complexes (EGFP-Cas9+gRNA) targeting MYO7A. Lanes 1 and 7 show size ladders. Lanes 2-5 show MYO7A^{sh1} amplicons incubated with EGFP-Cas9/gRNA RNP complexes comprising Cas9 associated with gRNA1, gRNA2, gRNA3, and gRNA4, respectively. Lane 6 shows an untreated MYO7A^{sh1} amplicon. The box labeled “Uncuts” indicates full-length MYO7A^{sh1} amplicons. The box labeled “Cuts” indicates cleaved fragments of MYO7A^{sh1} amplicons. The expected size of the full-length amplicon is ˜900 bp, and the expected sizes of the cleaved fragments are each 556-580 bp or 299-323 bp. These results demonstrate that EGFP-Cas9/gRNA RNP complexes carrying gRNA1, 2, 3, or 4 can each facilitate cleavage of MYO7A^{sh1} DNA. The primers used to amplify the MYO7A^{sh1} amplicons in this figure were forward

(SEQ ID NO: 31)
5′-GAGGGAACAGAGTGGCTATTAC-3′
and
reverse
(SEQ ID NO: 32)
5′-GCGTAGGAGTTGGACTTGATAG-3′.

FIGS. 6A-6B show electroporation-mediated transfection of primary fibroblast ear cells with EGFP protein (˜27 kDa). FIG. 6A shows optimization of electroporation parameters. FIG. 6B shows histogram flow cytometric analysis of electro-transfected cells with EGFP proteins. These results demonstrate that proteins can be transfected into these primary cells using optimized electroporation parameters.

FIGS. 7A-7B show contour plots of electroporation parameters pulse voltage (kV) and pulse width/duration (ms) versus cell viability (FIG. 7A) and EGFP transfection efficiency (FIG. 7B).

FIGS. 8A-8B show electroporation-mediated transfection of primary fibroblast car cells with EGFP-Cas9/gRNA RNP complexes. FIG. 8A shows optimization of electroporation parameters for EGFP-Cas9/gRNA RNP complexes. FIG. 8B shows fluorescent imaging of EGFP in fibroblast cells in suspension following electroporation protocol 3, 4, 5, and 6, respectively.

FIGS. 9A-9B show metrics of electro-transfection of Myo7a^{sh1/sh1} fibroblast cells with EGFP-Cas9 RNP complexes (prepared with a guide RNA having the nucleotide sequence AGGGAGAUGACGUUCAUAGG (SEQ ID NO: 42)); and Cy5-ODN (HDR template). FIG. 9A shows percent fluorescent Myo7A^{sh1/sh1} fibroblast cells (EGFP+, left; Cy5+, middle; and EGFP+/Cy5+, right) in samples of cells only, cells transfected with EGFP-Cas9, and cells transfected with EGFP-Cas9 and Cy5-ODN. The data show that about 90% of cells transfected with EGFP-Cas9 only were EGFP+, and about 20% of cells transfected with both EGFP-Cas9 and Cy5-ODN were EGFP+. About 80% of cells transfected with both EGFP-Cas9 and Cy5-ODN were Cy5+, and about 20% of cells transfected with both EGFP-Cas9 and Cy5-ODN were both EGFP+ and Cy5+. FIG. 9B shows percent EGFP+Myo7a^{sh1/sh1} fibroblast cells after electro-transfection with EGFP-Cas9/gRNA RNP complexes or EGFP-Cas9/gRNA RNP complexes and Cy5-ODN (HDR template) at different ratios. About 65% of cells transfected with only EGFP-Cas9/gRNA RNP complexes at 1× concentration were EGFP+, and about 10% of cells transfected with both EGFP-Cas9/gRNA RNP complexes at 1× concentration and Cy5-ODN at 1× concentration were EGFP+. Transfection of cells with 3× concentration of EGFP-Cas9/gRNA RNP complexes resulted in about 90% EGFP+ cells, and transfection with both 3×EGFP-Cas9/gRNA RNP complexes and 1×Cy5-ODN resulted in about 30% EGFP+ cells. Fluorescence was measured by flow cytometry.

FIG. 10 shows an electrophoresis gel of MYO7A^{sh1} amplicons following T7 endonuclease 1 (T7E1) assay of in vitro gene editing, prepared according to the workflow shown in FIG. 13. Lane 1 shows MYO7A^{sh1} amplicon without exposure to T7E1. Lane 2 shows MYO7A^{sh1} amplicon treated with T7E1 in the absence of gRNA. Lanes 3-7 show T7E1 digestion of MYO7A^{sh1} amplicons from cells treated with Cas9/gRNA RNP complexes prepared with gRNA1, gRNA2, gRNA3, gRNA4 and gRNA5, respectively. Stars indicate DNA fragments demonstrating desirable in vitro gene editing events. The results demonstrate that gRNA designs 1, 2, 3, and 4 are highly efficient at facilitating cleavage of MYO7A^{sh1}. The commercial gRNA (gRNA5) showed very low efficiency of cutting. The primers used to amplify the MYO7A^{sh1} amplicons in this figure were

forward
(SEQ ID NO: 31)
5′-GAGGGAACAGAGTGGCTATTAC-3′
and
reverse
(SEQ ID NO: 32)
5′-GCGTAGGAGTTGGACTTGATAG-3′.

FIG. 11 shows a chromatographic view of Sanger sequencing results of MYO7A^{sh1} gene amplicons without Cas9 treatment. Arrows labeled 1, 2, 3, 4, and 5 indicate the cutting sites for gRNA-1, 2, 3, 4, and 5, respectively. Sequence shown corresponds to SEQ ID NO: 54.

FIGS. 12A-12F show results of sequencing analysis of MYO7A^{sh1} gene amplicons following treatment with Cas9 and gRNAs. FIGS. 12A-12E show Sanger sequencing chromatograms of MYO7A^{sh1} amplicons following treatment with Cas9 and gRNA-1 (FIG. 12A), gRNA-2 (FIG. 12B), gRNA-3 (FIG. 12C), gRNA-4 (FIG. 12D), or commercial gRNA-5 (FIG. 12E). Arrows labeled 1, 2, 3, 4, and 5 indicate the cutting sites for gRNA-1, 2, 3, 4, and 5, respectively. The presence of minor peaks (i.e., corresponding to alternative nucleotides aside from those of the original nucleotide sequence) following each respective cut site in the chromatograms corresponding to gRNA-1, 2, 3, and 4 (FIGS. 12A, 12B, 12C, and 12D, respectively) demonstrate that each of these gRNAs was able to facilitate cleavage of MYO7A^{sh1} with Cas9. The absence of minor peaks in FIG. 12E indicate that gRNA-5 was not able to facilitate cleavage of MYO7A^{sh1}. Sequence shown corresponds to SEQ ID NO: 54. FIG. 12F shows the results of next-generation sequencing of MYO7A^{sh1} amplicons following treatment with Cas9 and gRNA-5, demonstrating poor cleavage efficiency of the commercial gRNA.

FIG. 13 shows the workflow for in vitro gene editing studies. ODN1 indicates the HDR template oligodeoxynucleotide designed for gRNA-1, -2, and -4, and ODN2 indicates the HDR template oligodeoxynucleotide specifically designed for gRNA-2 since the gRNA-2 site is more than 20 nt from the site of the MYO7A mutation.

FIG. 14 shows a workflow for electroporation-mediated transfection of extracellular vesicles with Cas9/gRNA RNP complexes and HDR template ODN and subsequent analysis.

FIGS. 15A-15B show schematics of the Myo7a^{sh1} gene locus. FIG. 15A shows a schematic of the single mutation in the Myo7a gene, pointing out the G1601C mutation in the gene sequence which results in the R502P substitution in the amino acid sequence of the encoded protein. The arrows at the bottom of the schematic show the sites to which the gRNA designs hybridize. Sequences shown (top-bottom) correspond to SEQ ID NOs: 60, 61, 62, 63, and 55. FIG. 15B shows Sanger sequencing confirming the presence of the Myo7a mutation in heterozygous Shaker-1 mutant mice (bold lower case letter is the mutant sequence). The DNA that was sequenced was isolated from fibroblast cells from car tissue of a heterozygous Myo7A^{WT/sh1} Shaker-1 mouse. Sequence shown corresponds to SEQ ID NO: 64.

FIGS. 16A-16B show results of a cell-free bioactivity assay of Cas9-RNP complexes. FIG. 16A shows an image of an agarose gel following electrophoresis of Myo7a amplicons amplified from homozygous Myo7a^{sh1/sh1} Shaker-1 mouse samples. FIG. 16B shows an image of an agarose gel following electrophoresis of Myo7a amplicons amplified from heterozygous Myo7a^{WT/sh1} Shaker-1 mouse samples. In both FIGS. 16A and 16B, the lanes from left to right show a 100 bp ladder; Myo7A amplicon without enzyme treatment; Myo7a amplicon treated with gRNA-1 Cas9 RNP complexes; Myo7a amplicon treated with Tru-gRNA-1 Cas9 RNP complexes; Myo7a amplicon treated with gRNA-2 Cas9 RNP complexes; and Myo7a amplicon treated with Tru-gRNA-2 Cas9 RNP complexes, respectively.

FIGS. 17A-17B show results of flow cytometric analysis of fibroblast cells following electroporation with different CRISPR constructs. FIG. 17A shows the percentage of EGFP+ cells in samples of cells only (Myo7a^{sh1/sh1} fibroblast cells) (left, circles; ˜0% EGFP+), cells transfected by electroporation with gRNA-1/EGFP-Cas9 RNP complexes (middle, squares; ˜65% EGFP+), and cells transfected by electroporation with Tru-gRNA-1/EGFP-Cas9 RNP complexes (right, triangles; ˜70% EGFP+). FIG. 17B shows the percentage of EGFP+ cells in samples of Myo7a^{sh1/sh1} (circles) or Myo7a^{WT/sh1} (triangles) fibroblasts without transfection (left; ˜0% EGFP+ for both Myo7a^{sh1/sh1} and Myo7a^{WT/sh1} cells) or after transfection by electroporation with EGFP-Cas9/gRNA-1 RNP complexes (right; ˜75% EGFP+ for Myo7a^{sh1/sh1} and ˜65% EGFP+ for Myo7a^{WT/sh1}).

FIGS. 18A-18C show in vitro gene editing efficiency by different gRNA/Cas9 RNP complexes in fibroblast cells. FIG. 18A shows an image of an agarose gel following electrophoresis of Myo7a amplicons amplified from homozygous Myo7a^{sh1/sh1} mouse samples.

FIG. 18B shows an image of an agarose gel following electrophoresis of Myo7a amplicons amplified from heterozygous Myo7a^{WT/sh1} mouse samples. FIG. 18C shows an image of an agarose gel following electrophoresis of Myo7a amplicons amplified from wild-type Myo7a^{WT/WT} mouse samples. In each gel shown in FIGS. 18A-18C, the lanes from left to right are 50 bp DNA ladder; Myo7a amplicon only; Myo7a amplicon treated with T7E1; Myo7a amplicon incubated with gRNA-1/Cas9 RNP complexes and treated with T7E1; Myo7a amplicon incubated with Tru-gRNA-1/Cas9 RNP complexes and treated with T7E1; Myo7a amplicon incubated with gRNA-2/Cas9 RNP complexes and treated with T7E1; and Myo7a amplicon incubated with Tru-gRNA-2/Cas9 RNP complexes and treated with T7E1, respectively. Editing efficiency is quantified in Table 2.

FIGS. 19A-19B show in vitro gene editing efficiency by RNP complexes produced with different guide RNAs. FIG. 19A shows quantification of gene editing efficiency measured by T7E1 assays. Each data point represents an independent electroporation of cells (fibroblasts from homozygous mutant Myo7a^{sh1/sh1} mice, circles, ˜24-45% indel formation in gRNA-transfected cells; heterozygous Myo7a^{WT/sh1} mice, triangles, ˜15-25% indel formation in gRNA-transfected cells; or homozygous wild-type Myo7a^{WT/WT} mice, diamonds, ˜0% indel formation). FIG. 19B shows quantification of gene editing efficiency measured by next-generation sequencing (NGS) of heterozygous Myo7a^{WT/sh1} fibroblast cells following transfection with RNP complexes produced with different guide RNAs (no RNP complexes, filled circles, ˜2% indels; gRNA-1, filled diamonds, ˜35% indel formation; gRNA-2, filled triangles, ˜35% indel formation; Tru-gRNA-1, open diamonds, ˜10% indel formation; or Tru-gRNA-2, open triangles, ˜20% indel formation).

FIG. 20 shows evaluation of types of mutations resulting from editing of mutant Myo7a by Cas9/gRNA RNP complexes in heterozygous Myo7a^{WT/sh1} fibroblast cells, as quantified by next-generation sequencing (NGS). In-frame shifts (left), frameshifts (middle), and non-coding mutations (right) were evaluated in cells transfected with Cas9 RNP complexes produced with gRNA-1 (filled circles labeled ‘1’; ˜75% in-frame shifts, ˜25% frameshifts, and 0% non-coding mutations), Tru-gRNA-1 (half-filled circles labeled ‘2’; ˜90% in-frame shifts, ˜10% frameshifts, and 0% non-coding mutations), gRNA-2 (filled diamonds labeled ‘3’; ˜ 15% in-frame shifts, ˜85% frameshifts, and 0% non-coding mutations), or Tru-gRNA-2 (half-filled diamonds, labeled ‘4’; ˜20% in-frame shifts, ˜80% frameshifts, and 0% non-coding mutations).

FIGS. 21A-21B show TIDE analysis of Sanger sequencing of DNA amplicons from gRNA-1/Cas9 RNP complex-treated heterozygous Myo7a^{WT/sh1} fibroblast cells. FIG. 21A shows a histogram of the percentage of sequences with different length insertions and deletions. The estimated overall gene editing efficiency was 17%. FIG. 21B shows decomposition analysis, with a significant increase in aberrant sequences following the expected cut site at the 553 bp position of the Myo7a amplicons.

FIGS. 22A-22B show TIDE analysis of Sanger sequencing of DNA amplicons from Tru-gRNA-1/Cas9 RNP complex-treated heterozygous Myo7a^{WT/sh1} fibroblast cells. FIG. 22A shows a histogram of the percentage of sequences with different length insertions and deletions. The estimated overall gene editing efficiency was 12.8%. FIG. 22B shows decomposition analysis, with a significant increase in aberrant sequences following the expected cut site at the 553 bp position of the Myo7a amplicons.

FIGS. 23A-23B show TIDE analysis of Sanger sequencing of DNA amplicons from gRNA-2/Cas9 RNP complex-treated heterozygous Myo7a^{WT/sh1} fibroblast cells. FIG. 23A shows a histogram of the percentage of sequences with different length insertions and deletions. The estimated overall gene editing efficiency was 23%. FIG. 23B shows decomposition analysis, with a significant increase in aberrant sequences following the expected cut site at the 548 bp position of the Myo7a amplicons.

FIGS. 24A-24B show TIDE analysis of Sanger sequencing of DNA amplicons from Tru-gRNA-2/Cas9 RNP complex-treated heterozygous Myo7a^{WT/sh1} fibroblast cells. FIG. 24A shows a histogram of the percentage of sequences with different length insertions and deletions. The estimated overall gene editing efficiency was 10.7%. FIG. 24B shows decomposition analysis, with a significant increase in aberrant sequences following the expected cut site at the 548 bp position of the Myo7a amplicons.

FIGS. 25A-25B show analysis of physical properties of extracellular vesicles (EVs) with or without CRISPR constructs. FIG. 25A shows nanoparticle tracking analysis (NanoSight) of the size distribution of untreated EVs (“Extracellular vesicles”) and EVs transfected with Cas9/gRNA RNP complexes by electroporation (“CrisprEVs”). FIG. 25B shows zeta potential analysis (LiteSizer 500) of untreated EVs (“EV only”) and EVs transfected with Cas9/gRNA RNP complexes by electroporation (“CrisprEV”).

FIGS. 26A-26B show quantification of loading efficiency of EVs with EGFP-Cas9/gRNA RNP complexes by electroporation (“CrisprEVs”) compared to untransfected EVs (“Empty EVs”) measured by nanoparticle tracking analysis. FIG. 26A shows the percentage of EGFP+ EVs. FIG. 26B shows the amount of EGFP-Cas9/gRNA RNP complexes quantified per 10⁸ EVs. (*, P<0.05)

### DETAILED DESCRIPTION

Gene therapy offers promising treatment options for certain genetic disorder, such as sensorineural hearing loss (SNHL), but current gene therapy methods have undesired toxicity and immunogenicity and suffer from poor delivery to the inner car.

The present disclosure is based in part on the development of CRISPR/Cas endonuclease (e.g., Cas9) compositions for the correction of SNHL-associated gene mutations, as well as compositions and methods for their delivery and use. Compared to previous gene therapy methods using viral vectors and virus-transduced hybridized vesicles, or using transfection methods such as those relying on nanoparticles or polymers, the disclosed compositions and methods possess greatly reduced toxicity and immunogenicity, and can protect gene therapy cargoes from degradation while also facilitating targeted delivery to inner ear hair cells. Conventional methods of therapeutic delivery, such as intratympanic injection and hydrogel delivery demonstrate poor therapeutic penetration beyond the blood-labyrinth barrier. The present disclosure provides compositions and formulations thereof with enhanced delivery to the inner car, as well as methods for using the same.

The present disclosure provides single guide RNAs (gRNAs) capable of facilitating correction of SNHL-associated gene mutations using CRISPR/Cas endonuclease (e.g., Cas9) and template nucleic acid, such as single-stranded DNA homology-directed repair (HDR) templates. Further, this disclosure provides extracellular vesicle (EV)-based delivery and therapy compositions and methods facilitating the use of gRNA/Cas endonuclease (e.g., Cas9) ribonucleoprotein (RNP) complexes and ssODN HDR templates for such gene therapy applications. As disclosed herein, the use of EVs, such as exosomes, which encapsulate gRNA/Cas endonuclease (e.g., Cas9) RNP complexes and ssODN HDR templates, enable correction of SNHL-associated gene mutations in vitro and in vivo. This can be achieved, for example, via EV-mediated delivery of gRNA/Cas endonuclease (e.g., Cas9) RNP complexes designed to cut a particular genomic locus and HDR templates to enable correction of mutations. EV-mediated delivery has the advantageous benefit of enabling efficient delivery of gene therapy cargoes (e.g., gRNA/Cas endonuclease (e.g., Cas9) RNP complexes and HDR templates disclosed herein) to the inner ear, including to inner ear hair cells. Exemplified herein are compositions and methods for correction of an SNHL-associated missense mutation in the MYO7A gene. Encompassed within the present disclosure are compositions and uses thereof for correction of other mutations associated with hearing loss.

According to some aspects of the present disclosure, methods and compositions for treating hearing disorders disclosed herein provide functional versions genes associated with hearing or by correcting mutations in such genes. In some embodiments, methods and compositions disclosed herein provide functional versions of genes associated with hearing to cells of the car, such as inner car hair cells. In some embodiments, methods and compositions disclosed herein facilitate correction of mutations in genes associated with hearing in cells of the car, such as inner car hair cells. In some embodiments, methods and compositions disclosed herein provide functional versions of MYO7A, or correct mutations in MYO7A.

In some embodiments, genes associated with hearing are provided to or corrected within a certain cell of a subject. In some embodiments, the cell is a hair cell. In some embodiments, the cell is an auditory hair cell. In some embodiments, the cell is a vestibular hair cell. In some embodiments, the cell is a cell of the organ of corti. In some embodiments, the cell is a hair cell of the organ of corti. In some embodiments, the cell is an inner cochlear hair cell. In some embodiments, the cell is an outer cochlear hair cell. In some embodiments, a mutation in a gene associated with hearing is corrected in a hair cell, such as an inner cochlear hair cell. In some embodiments, a mutation in MYO7A is corrected in a hair cell, such as an inner cochlear hair cell.

A mutation in a gene (e.g., a gene associated with hearing) can be corrected in a number of ways, such as through the use of nucleic acid editing proteins. In some embodiments, correction of a mutation in a gene as disclosed herein comprises the use of an endonuclease that is capable of cleaving a region in the endogenous mutated allele. In some embodiments, correction of a mutation in a gene comprises providing a template nucleic acid (e.g., a single-stranded oligodeoxynucleotide) with homology to the locus of the gene mutation and comprising a sequence with a corrected nucleotide sequence (i.e., comprising the non-mutated or wild-type sequence of the locus of the gene mutation). In some embodiments, correction of a mutation in a gene comprises the use of an endonuclease that is capable of cleaving a region in the endogenous mutated allele and providing a template nucleic acid. In some embodiments, correction of a mutation in a gene further comprises homology-directed repair (HDR) using the template nucleic acid. Through HDR, the mutated locus is corrected to match the sequence of the template nucleic acid, thereby correcting the mutation in the gene. Gene editing methods are generally classified based on the type of endonuclease that is involved in cleaving the target locus. Examples include, but are not limited to, Clustered Regularly Interspaced Short Palindromic Repeats (CRISPR)/CRISPR-associated (Cas) endonucleases (e.g., Cas9, Cas12a/Cpf1, and Cas13/C2c2), transcription activator-like effector-based nucleases (TALEN), zinc finger nucleases (ZFN), endonucleases (e.g., ARC homing endonucleases), meganucleases (e.g., mega-TALs), or a combination thereof. In some embodiments, correction of a mutation in a gene of a cell comprises delivering or otherwise providing a Cas endonuclease, a gRNA, and an HDR template nucleic acid to the cell. In some embodiments, correction of a mutation in MYO7A of a cell comprises delivering or otherwise providing a Cas endonuclease (e.g., Cas9), a gRNA (e.g., a gRNA disclosed herein), and a MYO7A HDR template nucleic acid (e.g., a template nucleic acid disclosed herein) to the cell.

Examples of endonucleases useful according to the present disclosure include, but are not limited to, Cas endonucleases (e.g., Cas9, Cas12a/Cpf1, and Cas13/C2c2), nickases (e.g., endonucleases which are only capable of cutting one strand of a double-stranded nucleic acid), and catalytically dead endonucleases (e.g., endonucleases that lack endonuclease activity, such as dCas9). Catalytically dead endonucleases are useful, for example, in CRISPR interference and CRISRP activation, wherein the catalytically dead endonuclease fused with a transcriptional effector to modulate target gene expression (e.g., to suppress or activate downstream gene expression). CRISPR interference and CRISPR activation are described in Jensen et al., “Targeted regulation of transcription in primary cells using CRISPRa and CRISPRi” Genome Res. 2021 31:2120-2130; doi: 10.1101/gr.275607.121. Accordingly, in embodiments described in this application in which Cas9 is specified, one or more alternative endonucleases (e.g., Cas nucleases described in this paragraph) can be used in place of Cas9.

Gene editing with CRISPR/Cas generally relies on at least two components: a gRNA that recognizes a target nucleic acid sequence and an endonuclease (e.g., Cas12a/Cpf1 or Cas9). A gRNA directs an endonuclease to a target site (e.g., a site within a gene associated with hearing), which typically contains a nucleotide sequence that is complementary (partially or completely) to the gRNA or a portion thereof. In some embodiments, the guide RNA is a two-piece RNA complex that comprises a protospacer fragment that is complementary to the target nucleic acid sequence and a scaffold RNA fragment. In some embodiments, the scaffold RNA is required to aid in recruiting the endonuclease to the target site. In some embodiments, the guide RNA is a single guide RNA that comprises both the protospacer sequence and the scaffold RNA sequence. An exemplary sequence of the scaffold RNA can be:

(SEQ ID NO: 33)
GUUUUAGAGCUAGAAAUAGCAAGUUAAAAUAAGGCUAGUCCGUUA
UCAACUUGAAAAAGUGGCACCGAGUCGGUGCUUUU.

Once at the target site, the endonuclease can generate a double strand break or a single-strand cut (a “nick”).

Nucleotide sequences for RNA molecules include residue “U.” The corresponding DNA sequence of any of the RNA sequences disclosed herein is also within the scope of the present disclosure. Such a DNA sequence would include “T” in replacement of “U” in the corresponding RNA sequence. One of ordinary skill in the art would understand that sequences disclosed herein which are described as RNA (e.g., “gRNA”) and which include “T” residues encompass the corresponding sequence comprising U's substituted for the T's, and vice versa (e.g., sequences comprising U's encompass the corresponding sequence comprising T's). As such, in any sequence disclosed herein (e.g., gRNA sequences, template sequences, target sequences, etc.), each uracil base (U) may independently and optionally be replaced with a thymine base (T) and each T may independently and optionally be replaced with a U.

The target nucleic acid for use with the CRISPR system is flanked on the 3′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, in some embodiments, for Cas9 endonucleases that are derived from Streptococcus pyogenes, the PAM sequence is NGG. In some embodiments, for Cas9 endonucleases derived from Staphylococcus aureus, the PAM sequence is NNGRRT. In some embodiments, for Cas9 endonucleases that are derived from Neisseria meningitidis, the PAM sequence is NNNNGATT. In some embodiments, for Cas9 endonucleases derived from Streptococcus thermophilus, the PAM sequence is NNAGAA (SEQ ID NO: 37). In some embodiments, for Cas9 endonuclease derived from Treponema denticola, the PAM sequence is NAAAAC. In some embodiments, for a Cpf1 nuclease, the PAM sequence is TTN. In this context, N represents A. G. T, or C. and R represents A or G, as would be recognized by one of ordinary skill in the art. Accordingly, in embodiments described in this application in which a PAM associated with a particular endonuclease is specified (e.g., in a gRNA sequence), one or more alternative PAM associated with a different endonuclease (e.g., a PAM associated with an endonuclease described in this paragraph) can be used in its place.

A CRISPR/Cas system that hybridizes with a target sequence in the locus of an endogenous gene may be used to modify the gene of interest (e.g., a mutated gene associated with hearing). In some embodiments, the nucleotide sequence that facilitates correction of a mutated gene is a gRNA that hybridizes to (i.e., is partially or completely complementary to) a target nucleic acid sequence in the mutated gene. For example, the gRNA or portion thereof may hybridize to the mutated gene with a hybridization region of between 15-25 nucleotides, 18-22 nucleotides, or 19-21 nucleotides in length. In some embodiments, the gRNA sequence that hybridizes to the mutated gene is 15, 16, 17, 18, 19, 20, 21, 22, 23, 24, or 25 nucleotides in length. In some embodiments, the gRNA sequence that hybridizes to the mutated gene is between 10-30, or between 15-25, nucleotides in length.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid such as a region in the mutated gene (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (see, e.g., Upadhyay, et al. Genes Genome Genetics (2013) 3(12):2233-2238). In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target region in the mutated gene (e.g., the last 5, 6, 7, 8, 9, or 10 nucleotides of the 3′ end of the target nucleic acid).

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the gRNA targets a gene associated with hearing, such as a gene comprising a mutation. In some embodiments, the gRNA targets MYO7A. In some embodiments, the gRNA comprises, consists essentially of, or consists of a nucleotide sequence of 10-30 or 15-25 consecutive nucleotides of, or a nucleotide sequence of 10-30 or 15-25 nucleotides capable of specifically binding to an equal length portion of the nucleotide sequence

(SEQ ID NO: 15)
CTGAGGGAACAGAGTGGCTATTACCAAGCCACTGCCTTCCAGGACCTC
CTTACCCACGTCCTCCCAACCTCCCCTGACTTTCTCAGTAGCTACCGC
CATCACACCCCGACACAGGCTCTTGGCCTCACTGCCAATCTGTGAGGT
GGGTGAACCACATAACCTTAGATGCACATTCCAGGCTAGGGCATCTGT
TTCTCATGACTAAAATGATTAGAAAGCTAGCGCTGGAGGACGTCTAGA
GCCCAGGAGTTCAAGGCTAGCCTGATCAATAGAGCAAGACTCTCTAAT
CTCACCGTTTCCCCCTGAGAGCAATACTATGTTTCCTTCCCCAGGTCA
AGCCAATTCTATCATTAGCATCTATCCTGAAGCAGCTGTGTAAATCTT
GATGGTTTGGCCCAGGGCCAGTGGGAAGCAGACAATCCCTGCTCCCCA
TGTGAACCCCCTAGAATCAGTGCAGAGCACCAGAACAGGCTCATGCGG
CTTCTCCCAGGTTGCAGATGCTGGGGGGAGCTGAGGCTTGCTGTGCCC
ACCTTGGGGAACTTGCTCTCCTCATCGATGAGGGAGATGACGTTCATA
GGCGGGTTGGCAATCATGTCCAGTGCTTCCTGGTTGTCAGTGAACTCA
ATGTGCAACCAGTCGATGCTCTCCAGGTCGTACTCCTCCTGCTCCAGC
TTGAACACGTGCCGCACGAAGAATTGCTGCAGGTGCTCATTGGCAAAG
TTAATGCAGAGCTGCTCGAAGCTGCAGAGGGAAGAGGACCTTGGACAT
GTGGCGCCCAACTTCTGCCCTGTCACCCAGACCCGGCTCTACCTAGAT
CACAGCTTGACACAAGACTCCCATCACGTGGGCTAGGGTACAACTATC
AAGTCCAACTCCTACGC.

In some embodiments, the gRNA comprises 1, 2, 3, 4, or 5 mismatches relative to the corresponding nucleotides of the sequence of SEQ ID NO: 15. In some embodiments, the gRNA comprises, consists essentially of, or consists of a nucleotide sequence of 10-30 or 15-25 consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises 1, 2, 3, 4, or 5 mismatches relative to the corresponding nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises, consists essentially of, or consists of a nucleotide sequence of 10-30 or 15-25 nucleotides capable of specifically hybridizing to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises 1, 2, 3, 4, or 5 mismatches relative to a nucleotide sequence of 10-30 or 15-25 nucleotides that is 100% complementary to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the gRNA comprises, consists essentially of, or consists of a nucleotide sequence of 10-30 or 15-25 consecutive nucleotides of a nucleotide sequence which encodes an amino acid sequence of NCBI Reference Sequence NP_001243010.1 (SEQ ID NO: 8), NP_001243011.1 (SEQ ID NO: 10), NP_001243012.1 (SEQ ID NO: 12), or NP_032689.2 (SEQ ID NO: 14). In some embodiments, the gRNA comprises 1, 2, 3, 4, or 5 mismatches relative to the corresponding nucleotides of a sequence which encodes an amino acid sequence of NCBI Reference Sequence NP_001243010.1 (SEQ ID NO: 8), NP_001243011.1 (SEQ ID NO: 10), NP_001243012.1 (SEQ ID NO: 12), or NP_032689.2 (SEQ ID NO: 14). In some embodiments, the gRNA comprises, consists essentially of, or consists of a nucleotide sequence of 10-30 or 15-25 nucleotides capable of specifically hybridizing to an equal-length portion of a nucleotide sequence which encodes an amino acid sequence of NCBI Reference Sequence NP_001243010.1 (SEQ ID NO: 8), NP_001243011.1 (SEQ ID NO: 10), NP_001243012.1 (SEQ ID NO: 12), or NP_032689.2 (SEQ ID NO: 14). In some embodiments, the gRNA comprises 1, 2, 3, 4, or 5 mismatches relative to the corresponding nucleotides of a sequence complementary to one which encodes an amino acid sequence of NCBI Reference Sequence NP_001243010.1 (SEQ ID NO: 8), NP_001243011.1 (SEQ ID NO: 10), NP_001243012.1 (SEQ ID NO: 12), or NP_032689.2 (SEQ ID NO: 14). Accordingly, in embodiments described in this application in which a particular gRNA (e.g., having a particular nucleotide sequence) is specified, one or more alternative gRNAs (e.g., as a gRNA described in this paragraph) can be used in its place.

In some embodiments, the gRNA comprises, consists essentially of, or consists of a nucleotide sequence of, or capable of specifically binding to any one of the sequences of

(SEQ ID NO: 16)
GATGACGTTCATAGGCCGGTTGG,
(SEQ ID NO: 17)
CTTGCTCTCCTCATCGATGAGGG,
(SEQ ID NO: 18)
ATGAGGGAGATGACGTTCATAGG,
(SEQ ID NO: 19)
AGGGAGATGACGTTCATAGGCGG,
or
(SEQ ID NO: 20)
CAATCATGTCCAGTGCTTCCTGG.

In some embodiments, the gRNA comprises, consists essentially of, or consists of a nucleotide sequence of, or capable of specifically binding to any one of the sequences of

(SEQ ID NO: 40)
GAUGACGUUCAUAGGCGGGU,
(SEQ ID NO: 41)
GACGUUCAUAGGCGGGU,
(SEQ ID NO: 42)
AGGGAGAUGACGUUCAUAGG,
(SEQ ID NO: 43)
GAGAUGACGUUCAUAGG,
(SEQ ID NO: 44)
CUUGCUCUCCUCAUCGAUGA,
or
(SEQ ID NO: 45)
AUGAGGGAGAUGACGUUCAU.

In some embodiments, the gRNA comprises, consists essentially of, or consists of a nucleotide sequence capable of specifically hybridizing to a nucleotide sequence of

(SEQ ID NO: 21)
CCAACCGGCCTATGAACGTCATC,
(SEQ ID NO: 22)
CCCTCATCGATGAGGAGAGCAAG,
(SEQ ID NO: 23)
CCTATGAACGTCATCTCCCTCAT,
(SEQ ID NO: 24)
CCGCCTATGAACGTCATCTCCCT,
or
(SEQ ID NO: 25)
CCAGGAAGCACTGGACATGATTG.

In some embodiments, the gRNA comprises, consists essentially of, or consists of a nucleotide sequence capable of specifically hybridizing to a nucleotide sequence of

(SEQ ID NO: 46)
ACCCGCCTATGAACGTCATC,
(SEQ ID NO: 47)
ACCCGCCTATGAACGTC,
(SEQ ID NO: 48)
CCTATGAACGTCATCTCCCT,
(SEQ ID NO: 49)
CCTATGAACGTCATCTC,
or
(SEQ ID NO: 50)
TCATCGATGAGGAGAGCAAG.

In some embodiments, the gRNA does not comprise a nucleotide sequence of CAATCATGTCCAGTGCTTCCTGG (SEQ ID NO: 20) or a nucleotide sequence capable of specifically hybridizing to a nucleotide sequence of CCAGGAAGCACTGGACATGATTG (SEQ ID NO: 25). In some embodiments, the gRNA comprises, consists essentially of, or consists of a nucleotide sequence of 10-30 or 15-25 (e.g., 15, 16, 17, 18, 19, 20, 21, 22, 23, 24, or 25) consecutive nucleotides of or capable of specifically hybridizing to

(SEQ ID NO: 26)
TCATCGATGAGGGAGATGACGTTCATAGGCGGGTTGGCAATCATG
TCCAGTGCTTCCTGGT.

In some embodiments, the gRNA that targets the mutated gene comprises, consists essentially of, or consists of a nucleotide sequence of or capable of specifically hybridizing to a nucleotide sequence of 10-30 or 15-25 (e.g., 15, 16, 17, 18, 19, 20, 21, 22, 23, 24, or 25) consecutive nucleotides of

(SEQ ID NO: 27)
ACCAGGAAGCACTGGACATGATTGCCAACCCGCCTATGAACGTCA
TCTCCCTCATCGATGA.

It should be understood that while sequences disclosed herein are shown with T or U nucleotides, both RNA and DNA sequences are contemplated, such that a sequence disclosed herein comprising T's can also be provided or used with U's in place of the T's, and a sequence comprising U's can also be provided or used with T's in place of the U's. As such, in sequences disclosed herein, each (e.g., one or more) uracil base (U) may independently and optionally be replaced with a thymine base (T) and each (e.g., one or more) T may independently and optionally be replaced with a U. For example, one or more (e.g., all) of the U's in a given sequence can be substituted with T's, and one or more (e.g., all) of the T's in a given sequence can be substituted with U's.

In some embodiments, a sequence (e.g., a gRNA sequence) that is “capable of specifically hybridizing to” or “capable of specifically binding to” another sequence is the reverse complement of that sequence, or has at least 70% sequence identity with the reverse complement of that sequence.

In some embodiments, a gRNA disclosed herein has at least 70% (e.g., at least 75%, 80%, 85%, 90%, 91%, 92%, 93%, 94%, 95%, 96%, 97%, 98%, 99%, or 100%) sequence homology to a nucleotide sequence disclosed herein (e.g., any one of SEQ ID NOs: 16-27 and 40-50). In some embodiments, a gRNA disclosed herein comprises 1, 2, 3, 4, or 5 mismatches relative to a nucleotide sequence disclosed herein (e.g., any one of SEQ ID NOs: 16-27 and 40-50). In some embodiments, a gRNA disclosed herein has at least 70% (e.g., at least 75%, 80%, 85%, 90%, 91%, 92%, 93%, 94%, 95%, 96%, 97%, 98%, 99%, or 100%) sequence homology to a nucleotide sequence disclosed herein (e.g., a nucleotide sequence of 10-30 or 15-25 consecutive nucleotides of, or capable of specifically hybridizing to an equal-length portion of any one of SEQ ID NOs: 1, 3, 5, 7, 9, 11, 13, or 15). In some embodiments, a gRNA disclosed herein comprises 1, 2, 3, 4, or 5 mismatches relative to a nucleotide sequence disclosed herein (e.g., a nucleotide sequence of 10-30 or 15-25 consecutive nucleotides of, or capable of specifically hybridizing to an equal-length portion of any one of SEQ ID NOs: 1, 3, 5, 7, 9, 11, 13, or 15).

In some embodiments, a gRNA disclosed herein targets a human MYO7A sequence. In some embodiments, a gRNA disclosed herein targets a human MYO7A sequence comprising a mutation, such as a mutation which causes or is associated with hearing loss. In some embodiments, the gRNA comprises, consists essentially of, or consists of a nucleotide sequence of 10-30 or 15-25 consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5). In some embodiments, the gRNA comprises 1, 2, 3, 4, or 5 mismatches relative to the corresponding nucleotides of the sequence of NCBI Reference Sequence NM_000260.4 (SEQ ID NO: 1), NM_001127180.2 (SEQ ID NO: 3), or NM_001369365.1 (SEQ ID NO: 5). In some embodiments, the gRNA comprises, consists essentially of, or consists of a nucleotide sequence of 10-30 or 15-25 nucleotides capable of specifically hybridizing to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5). In some embodiments, the gRNA comprises 1, 2, 3, 4, or 5 mismatches relative to a nucleotide sequence of 10-30 or 15-25 nucleotides that is 100% complementary to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5). In some embodiments, the gRNA comprises, consists essentially of, or consists of a nucleotide sequence of 10-30 or 15-25 consecutive nucleotides of a nucleotide sequence which encodes an amino acid sequence of NCBI Reference Sequence NP_000251.3 (SEQ ID NO: 2), NP_001120652.1 (SEQ ID NO: 4), or NP_001356294.1 (SEQ ID NO: 6). In some embodiments, the gRNA comprises 1, 2, 3, 4, or 5 mismatches relative to the corresponding nucleotides of a sequence which encodes an amino acid sequence of NCBI Reference Sequence NP_000251.3 (SEQ ID NO: 2), NP_001120652.1 (SEQ ID NO: 4), or NP_001356294.1 (SEQ ID NO: 6). In some embodiments, the gRNA comprises, consists essentially of, or consists of a nucleotide sequence of 10-30 or 15-25 nucleotides capable of specifically hybridizing to an equal-length portion of a nucleotide sequence which encodes an amino acid sequence of NCBI Reference Sequence NP_000251.3 (SEQ ID NO: 2), NP_001120652.1 (SEQ ID NO: 4), or NP_001356294.1 (SEQ ID NO: 6). In some embodiments, the gRNA comprises 1, 2, 3, 4, or 5 mismatches relative to the corresponding nucleotides of a sequence complementary to one which encodes an amino acid sequence of NCBI Reference Sequence NP_000251.3 (SEQ ID NO: 2), NP_001120652.1 (SEQ ID NO: 4), or NP_001356294.1 (SEQ ID NO: 6). The nucleotide and amino acid sequences of NCBI Reference Sequences described herein are provided in the Sequences section below.

In some embodiments, a gRNA disclosed herein targets a specific allele of a gene (e.g., a specific allele of MYO7A, such as a mutant allele of MYO7A). A gRNA targeting a specific allele of a gene may comprise a sequence that is complementary to a portion of the allele comprising a mutation (e.g., a single nucleotide mutation, such as a one giving rise to an amino acid substitution) such that the gRNA targets only the allele comprising the mutation. In some embodiments, the portion of the gRNA sequence that is complementary to a portion of the allele comprising a mutation is near the 3′ end of the gRNA sequence (e.g., within 1, 2, 3, 4, 5, 6, 7, or 8 nucleotides of the 3′ end of the gRNA sequence).

In some embodiments, a gRNA and a CRISPR-associated (Cas) endonuclease (e.g., Cas9, Cas12a/Cpf1, or Cas13/C2c2), are combined to form a ribonucleoprotein (RNP) complex. In some embodiments, an RNP complex comprises a gRNA disclosed herein associated with a Cas endonuclease (e.g., Cas9, Cas12a/Cpf1, or Cas13/C2c2). In some embodiments, an RNP complex comprises or consists of a Cas endonuclease and a guide RNA (e.g., a guide RNA disclosed herein, optionally including a scaffold RNA sequence in addition to a Cas endonuclease/gRNA RNP complexes can be formed by methods known in the art, such as by incubating a gRNA with a Cas endonuclease (e.g., at room temperature) such that complexes are formed. gRNAs, RNP complexes, Cas endonucleases, and methods of their preparation and use are described in International Patent Application Publication Nos. WO2014018423A2, WO2014093661A2, WO2016205764A1, WO2018213708A1, the entire contents of each of which are herein incorporated by reference. Accordingly, in embodiments described in this application in which a particular gRNA and a particular endonuclease are specified in a given RNP complex, one or more alternative gRNAs (e.g., a gRNA described herein) and/or one or more alternative endonucleases (e.g., an endonuclease described herein) can be used in place of the particular gRNA and/or the particular endonuclease.

Mutations in genes associated with hearing (e.g., MYO7A) are associated with a number of diseases, disorders, and conditions that may be treated by the use of methods and compositions disclosed herein. In some embodiments, the disease, disorder, or condition is a hearing loss disorder. Hearing loss disorders can be characterized by one or more of total or partial loss of hearing; tinnitus; decreased ability to hear or perceive certain sounds (e.g., certain frequencies of sound or certain amplitudes of sound); increased sensitivity to certain sounds (e.g., sensitivity to loud sounds or sounds of certain frequencies); and/or vestibular dysfunction (e.g., balance problems, disorientation, vertigo, or dizziness). Hearing loss disorders include, but are not limited to sensorineural hearing loss (SNHL) disorders, Usher syndrome, and nonsyndromic hearing loss (e.g., autosomal dominant deafness-11 (DFNA11) and autosomal recessive nonsyndromic deafness-2 (DFNB2)). Symptoms of hearing loss disorders can be congenital or can develop during childhood or later in life (e.g., from months of age through childhood, during adolescence, or in adulthood). In some instances hearing loss disorders have additional symptoms, such as vision problems or vision loss, retinitis pigmentosa, and retinal dystrophy. Examples of mutations in genes associated with hearing and their symptoms are described in Gibson et al. Nature 374:62-64 (1995); Guilford et al. Hum. Molec. Genet. 3:989-993 (1994); Hildebrand et al. Clin. Genet. 77:563-571 (2010); Liu et al. Nature Genet. 16:188-190 (1997); Liu et al. Nature Genet. 17:268-269 (1997); Riazuddin et al. Hum. Mutat. 29:502-511 (2008); Weil et al. Nature 374: 60-61 (1995); Weil et al. Nature Genet. 16:191-193 (1997); Weil et al. Proc. Nat. Acad. Sci. USA 93:3232-3237 (1996); Zina et al. Am. J. Med. Genet. 101:181-183 (2001); Tamagawa et al. Hum. Molec. Genet. 5:849-852 (1996); Cuevas et al. Molec. Cell. Probes 12:417-420 (1998); Janecke et al. Hum. Mutat. 13:133-140 (1999); Kelley et al. Genomics 40:73-79 (1997); Levy et al. Hum. Molec. Genet. 6:111-116 (1997); Ouyang et al. Hum. Genet. 116:292-299 (2005); Sun et al. J. Hum. Genet. 56:64-70 (2011); and Weston et al. Am. J. Hum. Genet. 59:1074-1083 (1996).

In some embodiments, the gRNA that targets the mutated gene comprises one or more modifications, such as internucleoside linkage modifications, sugar modifications, or base modifications. In some embodiments, the gRNA that targets the mutated gene comprises one or more phosphorothioate internucleoside linkages. In some embodiments, the gRNA that targets the mutated gene comprises one or more 2′-O-methyl modified nucleotides. In some embodiments, the gRNA that targets the mutated gene comprises one or more phosphorothioate internucleoside linkages and one or more 2′-O-methyl modified nucleotides. In some embodiments, the gRNA that targets the mutated gene comprises three consecutive 2′-O-methyl modified nucleotides at the 5′ end, three consecutive 2′-O-methyl modified nucleotides at the 3′ end, or three consecutive 2′-O-methyl modified nucleotides at both the 5′ end and the 3′ end. In some embodiments, the gRNA that targets the mutated gene comprises three consecutive phosphorothioate internucleoside linkages at the 5′ end, three consecutive phosphorothioate internucleoside linkages at the 3′ end, or three consecutive phosphorothioate internucleoside linkages at both the 5′ end and the 3′ end. In some embodiments, the gRNA that targets the mutated gene comprises three consecutive 2′-O-methyl modified nucleotides and three consecutive internucleoside linkages modifications at both the 5′ end and the 3′ end.

In some embodiments, Cas endonucleases are modified relative to their wild-type sequences. A variety of Cas endonucleases are known in the art and modifications are regularly made, and numerous references describe rules and parameters that are used to guide the design of Cas systems (e.g., including Cas9 target selection tools). Sec, e.g., Hsu et al., Cell, 157(6): 1262-78, 2014. In some embodiments, the Cas endonuclease is modified to include a nuclear localization signal, an SV40 tag, or a nucleoplasmin nuclear localization signal.

As disclosed herein, a “template nucleic acid” refers to a nucleic acid molecule for use in a gene editing method. A template nucleic acid typically comprises a nucleotide sequence of a reference or wild-type gene, such as a wild-type MYO7A gene. A template nucleic acid may in some embodiments comprise a nucleotide sequence designed to introduce a premature stop codon into an allele of a gene. For example, a template nucleic acid designed to introduce a premature stop codon into an allele of a gene in some embodiments comprises flanking sequences with homology to an allele of the gene and a medial sequence encoding a stop codon. A template nucleic acid can in some embodiments be used as a homology-directed repair (HDR) template, such as to correct a mutation in a gene. A template nucleic acid can in some embodiments be used to edit a gene through a non-homology dependent method, such as homology-independent targeted integration (HITI). Gene editing methods involving template nucleic acids are described, for example, in Yeh et al., Nature Cell Biology 21:1468-1478 (2019) and Suzuki & Izpisua Belmonte, J. Hum. Genet. 63:157-164 (2018). In some embodiments, a template nucleic acid is single-stranded. In some embodiments, a template nucleic acid is a single-stranded oligonucleotide (e.g., an oligodeoxynucleotide or oligoribonucleotide). In some embodiments, a template nucleic acid is double-stranded. In some embodiments, a template nucleic acid is a double-stranded oligonucleotide (e.g., an oligodeoxynucleotide or oligoribonucleotide). In some embodiments, a template nucleic acid (e.g., a template nucleic acid exogenous to the cell in which a gene is to be edited) is not used in a gene editing method disclosed herein.

In some embodiments, the template nucleic acid for correcting the mutated gene comprises, consists essentially of, or consists of a nucleotide sequence of 50-120 (e.g., 50, 55, 60, 65, 70, 71, 72, 73, 74, 75, 76, 77, 78, 79, 80, 85, 90, 95, 100, 105, 110, 115, or 120) consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the template nucleic acid for correcting the mutated gene comprises, consists essentially of, or consists of a nucleotide sequence of 50-120 (e.g., 50, 55, 60, 65, 70, 71, 72, 73, 74, 75, 76, 77, 78, 79, 80, 85, 90, 95, 100, 105, 110, 115, or 120) nucleotides capable of specifically binding to an equal length nucleotide sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13). In some embodiments, the template nucleic acid for correcting the mutated gene comprises, consists essentially of, or consists of a nucleotide sequence of

(SEQ ID NO: 28)
AACCAGGAAGCACTGGACATGATTGCCAACCGGCCTATGAACGTCATCTC
CCTCATCGATGAGGAGAGCAAG;
(SEQ ID NO: 29)
AATCAAGAAGCACTGGACATGATTGCCAATCGGCCVATGAACGTCATCTC
DCTCATCGATGAGGAGAGCAAG, wherein V is A, C, or G
and D is A, G, or T;
(SEQ ID NO: 30)
ACAACCAGGAAGCACTGGACATGATTGCCAACCGGCCTATGAACGTCATC
TCACTCATTGATGAAGAGAGCAAGTTCCCCAAGGTGGGCACAGCAA;
(SEQ ID NO: 51)
TGAGTTCACTGACAACCAGGAAGCACTGGACATGATTGCCAATCGGCCAA
TGAACGTCATCTCGCTCATCGATGAGGAGAGCAAGTTCCCCAAGGT;
or
(SEQ ID NO: 52)
TTGCTGTGCCCACCTTGGGGAACTTGCTCTCTTCATCAATGAGTGAGATG
ACGTTCATAGGCCGGTTGGCAATCATGTCCAGTGCTTCCTGGTTGT.

In some embodiments, the template nucleic acid for correcting the mutated gene comprises, consists essentially of, or consists of a nucleotide sequence capable of specifically binding to

(SEQ ID NO: 28)
AACCAGGAAGCACTGGACATGATTGCCAACCGGCCTATGAACGTCATCTC
CCTCATCGATGAGGAGAGCAAG;
(SEQ ID NO: 29)
AATCAAGAAGCACTGGACATGATTGCCAATCGGCCVATGAACGTCATCTC
DCTCATCGATGAGGAGAGCAAG, wherein V is A, C, or G
and D is A, G, or T;
(SEQ ID NO: 30)
ACAACCAGGAAGCACTGGACATGATTGCCAACCGGCCTATGAACGTCATC
TCACTCATTGATGAAGAGAGCAAGTTCCCCAAGGTGGGCACAGCAA;
(SEQ ID NO: 51)
TGAGTTCACTGACAACCAGGAAGCACTGGACATGATTGCCAATCGGCCAA
TGAACGTCATCTCGCTCATCGATGAGGAGAGCAAGTTCCCCAAGGT;
or
(SEQ ID NO: 52)
TTGCTGTGCCCACCTTGGGGAACTTGCTCTCTTCATCAATGAGTGAGATG
ACGTTCATAGGCCGGTTGGCAATCATGTCCAGTGCTTCCTGGTTGT.

In some embodiments, the template nucleic acid for correcting the mutated gene comprises, consists essentially of, or consists of a nucleotide sequence of 50-100 (e.g., 50, 55, 60, 65, 70, 71, 72, 73, 74, 75, 76, 77, 78, 79, 80, 85, 90, 95, or 100) consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5). In some embodiments, the template nucleic acid for correcting the mutated gene comprises, consists essentially of, or consists of a nucleotide sequence of 50-100 (e.g., 50, 55, 60, 65, 70, 71, 72, 73, 74, 75, 76, 77, 78, 79, 80, 85, 90, 95, or 100) nucleotides capable of specifically binding to an equal length nucleotide sequence of NCBI Reference Sequence NM_000260.4 (SEQ ID NO: 1), NM_001127180.2 (SEQ ID NO: 3), or NM_001369365.1 (SEQ ID NO: 5). In some embodiments, the template nucleic acid for correcting the mutated gene comprises a substituted nucleotide relative to the wild-type sequence which represents a silent mutation in the nucleotides comprising the PAM sequence.

In some embodiments, extracellular vesicles encapsulating gRNAs, endonuclease (e.g., CRISPR-associated endonucleases, including but not limited to Cas9) proteins, gRNA/endonuclease (e.g., CRISPR-associated endonuclease) RNP complexes, template nucleic acids, or combinations thereof, are disclosed herein. Extracellular vesicles include exosomes, ectosomes, microvesicles, and microparticles. Extracellular vesicles (EVs) are particles delineated by a lipid bilayer encapsulating cytosol-like material, which are released from a cell but that lack a nucleus. EVs range in size from 20-30 nm in diameter to as large as 10 μm in diameter or more, however most EVs are 200 nm or less in diameter. EVs typically comprise various biological cargoes derived from the parent cell, including proteins, nucleic acids, lipids, metabolites, and in some instances organelles. Exosomes are EVs of endosomal origin, and are produced by pinching off of an invagination of an inward budding of an endosome membrane, followed by fusion of the endosome with the cell membrane, thereby releasing the exosome. Exosomes are typically 30 to 150 nm in diameter. In some embodiments, EVs disclosed herein are manipulated such that they comprise a gRNA, an endonuclease (e.g., a Cas endonuclease), a gRNA/endonuclease RNP complex, a template nucleic acid, or a combination thereof.

EVs, including exosomes, can be isolated from various sources, including cell culture supernatant and biological fluids (e.g., blood). In some embodiments, EVs (e.g., exosomes) disclosed herein are isolated from cell culture supernatant. In some embodiments, EVs (e.g., exosomes) are isolated from auditory cells (e.g., from cultures of primary cells or cell lines isolated or otherwise derived from the car). In some embodiments, EVs (e.g., exosomes) are isolated from cells of the car (e.g., from cultures of cells isolated or otherwise derived from the car, such as from the organ of corti). In some embodiments, EVs (e.g., exosomes) are isolated from hair cells (e.g., from cultures of hair cells). In some embodiments, EVs (e.g., exosomes) disclosed herein are isolated from cultures of HEI-OC1 cells.

In some embodiments, EVs (e.g., exosomes) disclosed herein comprise a surface molecule (e.g., a receptor or ligand protein) present on or capable of binding to a hair cell. In some embodiments, EVs (e.g., exosomes) disclosed herein comprise a surface molecule derived from a hair cell. In some embodiments, EVs (e.g., exosomes) disclosed herein comprise a surface marker characteristic of hair cells. In some embodiments, EVs (e.g., exosomes) disclosed herein comprise or express one or more of Nestin, Abcg2, Pax-2, BMP-4, BMP-7, MYO7A, Espin, Brn3C, Atoh1, Anxa4a, Calretinin (Calb2), Sox2, F-actin, prestin, HSP70, integrin, Tmc1, and P27^{kip1}. In some embodiments, EVs (e.g., exosomes) disclosed herein comprise or express one or more of Nestin, prestin, HSP70, integrin, and Tmc1. In some embodiments, EVs (e.g., exosomes) disclosed herein comprise one or more surface molecules capable of facilitating binding to or internalization by a hair cell.

In some embodiments, gRNAs, Cas proteins (e.g., Cas9 proteins), gRNA/Cas (e.g., Cas9) ribonucleoprotein (RNP) complexes, and/or template nucleic acids disclosed are encapsulated within EVs (e.g., exosomes). In some embodiments, encapsulation is achieved by electroporation of a plurality of EVs (e.g., exosomes) in a solution comprising gRNAs, Cas proteins (e.g., Cas9 proteins), gRNA/Cas (e.g., Cas9) RNP complexes, and/or template nucleic acids disclosed herein. In some embodiments, a gRNA/Cas (e.g., Cas9) RNP complex and a template nucleic acid disclosed herein are encapsulated within an EV (e.g., exosome). In some embodiments, a gRNA/Cas (e.g., Cas9) RNP complex and a template nucleic acid disclosed herein are encapsulated within an EV (e.g., exosome) by electroporation of the EV in the presence of the gRNA/Cas (e.g., Cas9) RNP complex and the template nucleic acid. Electroporation involves applying an electrical field to a sample (e.g., an EV), thereby increasing the permeability of the cell membrane and allowing molecules (e.g., nucleic acids, proteins, or small molecules) to be introduced into the cell, either passively or by electrophoresis (for charged molecules). The voltage and duration of the applied electric pulse affect the outcome of the electroporation, both determining the viability of the resultant product and the loading efficiency of the molecules of interest. In some embodiments, electroporation (e.g., to load gRNA/Cas (e.g., Cas9) RNP complexes and/or template nucleic acids into EVs) comprises the use of an electric pulse having a voltage of less than 2000V (e.g., less than 1900V, less than 1850V, less than 1800V, less than 1750V, less than 1700V, less than 1650V, less than 1600V, less than 1550V, less than 1500V, less than 1450V, less than 1400V, less than 1350V, less than 1300V, less than 1250V, less than 1200V, less than 1150V, less than 1100V, less than 1050V, less than 1000V, less than 900V, less than 800V, less than 700V, less than 600V, or less than 500V). In some embodiments, the voltage of the electric pulse is or is about 500V, 600V, 700V, 800V, 900V, 1000V, 1050V, 1100V, 1150V, 1200V, 1250V, 1300V, 1350V, 1400V, 1450V, 1500V, 1550V, 1600V, 1650V, 1700V, 1750V, 1800V, 1850V, 1900V, or 2000V. In some embodiments, the voltage of the electric pulse is between about 1200V and about 1750V. In some embodiments, the voltage of the electric pulse is between about 1250V and about 1650V. In some embodiments, the voltage of the electric pulse is between about 1400V and about 1600V. In some embodiments, the voltage of the electric pulse is between about 1450V and about 1550V. In some embodiments, the voltage of the electric pulse is or is about 1450V. In some embodiments, the voltage of the electric pulse is or is about 1500V. In some embodiments, the voltage of the electric pulse is or is about 1550V. In some embodiments, electroporation (e.g., to load gRNA/Cas endonuclease (e.g., Cas9) complexes and/or template nucleic acids into EVs) comprises the use of an electric pulse less than 50 ms in duration (e.g., less than 45 ms, less than 40 ms, less than 35 ms, less than 30 ms, less than 25 ms, less than 20 ms, less than 15 ms, or less than 10 ms). In some embodiments, the duration of the electric pulse is or is about 10 ms, 15 ms, 20 ms, 25 ms, 30 ms, 35 ms, 40 ms, 45 ms, or 50 ms. In some embodiments, the duration of the electric pulse is between about 15 ms and about 40 ms. In some embodiments, the duration of the electric pulse is between about 20 ms and about 35 ms. In some embodiments, the duration of the electric pulse is between about 20 ms and about 30 ms. In some embodiments, the duration of the electric pulse is between about 25 ms and about 35 ms. In some embodiments, the duration of the electric pulse is between about 25 ms and about 30 ms. In some embodiments, the duration of the electric pulse is or is about 15 ms. In some embodiments, the duration of the electric pulse is or is about 20 ms. In some embodiments, the duration of the electric pulse is or is about 25 ms. In some embodiments, the duration of the electric pulse is or is about 30 ms. In some embodiments, the duration of the electric pulse is or is about 35 ms.

In some embodiments, an additional agent is added to extracellular vesicles (e.g., exosomes). In some embodiments, the additional agent improves stability of the EVs (e.g., exosomes). In some embodiments, the additional agent is a stabilizing agent. In some embodiments, the additional agent is added to the EVs (e.g., exosomes) prior to electroporation. In some embodiments, the additional agent is added to the EVs (e.g., exosomes) at the time of electroporation. In some embodiments, the additional agent is added to the EVs (e.g., exosomes) after electroporation. In some embodiments, the additional agent is a stabilizing agent. In some embodiments, the additional agent is a sugar. In some embodiments, the additional agent is a compound sugar. In some embodiments, the additional agent is a disaccharide (i.e., containing 2 monosaccharides). In some embodiments, the additional agent is an oligosaccharide containing 3-10 monosaccharides. In some embodiments, the additional agent is sucrose, trehalose, lactose, maltose, cellobiose, chitobiose, kojibiose, nigerose, isomaltose, β,β-trehalose, α,β-trehalose, sophorose, laminaribiose, gentiobiose, trehalulose, turanose, maltulose, leucrose, isomaltulose, gentiobiulose, mannobiose, melibiose, melibiulose, rutinose, rutinulose, or xylobiose. In some embodiments, the additional agent is trehalose.

Methods and compositions provided herein can be used for correcting mutations in genes associated with hearing. In some embodiments, the gene to be corrected (e.g., a gene comprising a mutation) using methods or compositions disclosed herein is ACTG1, CDH23, CLDN14, COCH, COL11A2, DFNA5, ESPN, EYA4, GJB2, GJB6, GRXCR1, KCNQ4, MYO3A, MYO15A, MY06, MYO7A, OTOF, OTOA, PCDH15, POU3F4, RDX, SLC26A4, STRC, TECTA, TMC1, TMIE, TMPRSS3, USH1C, WFS1, WHRN, CCDC50, DIAPH1, DSPP, ESRRB, GJB3, GRHL2, HGF, LHFPL5, LOXHD1, LRTOMT, MARVELD2, MIR96, MYH14, MYH9, MYO1A, PJVK, POU4F3, PRPS1, PTPRQ, SERPINB6, SIX1. SLC17A8, TPRN, or TRIOBP. In some embodiments, the gene to be corrected is ACTG1, CDH23, CLDN14, COCH, COL11A2, DFNA5, ESPN, EYA4, GJB2, GJB6, GRXCR1, KCNQ4, MYO3A, MYO15A, MY06, MYO7A, OTOF, OTOA, PCDH15, POU3F4, RDX, SLC26A4, STRC, TECTA, TMC1, TMIE, TMPRSS3, USH1C, WFS1, or WHRN. In some embodiments, the gene to be corrected is MYO7A. Accordingly, in some embodiments methods described herein can be used with a gRNA that targets one of ACTG1, CDH23, CLDN14, COCH, COL11A2, DFNA5, ESPN, EYA4, GJB2, GJB6, GRXCR1, KCNQ4, MYO3A, MYO15A, MY06, MYO7A, OTOF, OTOA, PCDH15, POU3F4, RDX, SLC26A4, STRC, TECTA, TMC1, TMIE, TMPRSS3, USH1C, WFS1, WHRN, CCDC50, DIAPH1, DSPP, ESRRB, GJB3, GRHL2, HGF, LHFPL5, LOXHD1, LRTOMT, MARVELD2, MIR96, MYH14, MYH9, MYO1A, PJVK, POU4F3, PRPS1, PTPRQ. SERPINB6, SIX1, SLC17A8, TPRN, or TRIOBP. In some embodiments, a gRNA targeting one of the genes listed above facilitates cleavage of the gene within 50 (e.g., within 45, 40, 35, 30, 25, 20, 15, 10, 5, 4, 3, 2, or 1) nucleotides of the site of the mutation. In some embodiments, a gRNA targeting one of the genes listed above facilitates cleavage of the gene within 30 or fewer nucleotides of the site of the mutation. In some embodiments, a gRNA targeting one of the genes listed above facilitates cleavage of the gene within 20 nucleotides of the site of the mutation. In some embodiments, a gRNA targeting one of the genes listed above facilitates cleavage of the gene within 10 nucleotides of the site of the mutation.

Methods and compositions provided herein can be used for treating a disease, disorder, or condition in a subject in need thereof. In some embodiments, the disease, disorder, or condition is hearing loss. In some embodiments, the disease, disorder, or condition is SNHL. In some embodiments, a subject in need of treatment is a patient who has or is suspected of having hearing loss (e.g., SNHL). In some embodiments, a subject in need of treatment is a patient who has been diagnosed with hearing loss (e.g., SNHL). In some embodiments, a subject in need of treatment is a human patient. In some embodiments, a subject in need of treatment is a patient in whom a mutation in a gene associated with hearing has been identified, for example by exome, whole genome, or gene-specific sequencing. In some embodiments, a subject in need of treatment is a patient in whom a mutation in ACTG1, CDH23, CLDN14, COCH, COL11A2, DFNA5, ESPN, EYA4, GJB2, GJB6, GRXCR1, KCNQ4, MYO3A, MYO15A, MY06, MYO7A, OTOF, OTOA, PCDH15, POU3F4, RDX, SLC26A4, STRC, TECTA, TMC1, TMIE, TMPRSS3, USH1C, WFS1, WHRN, CCDC50, DIAPH1, DSPP, ESRRB, GJB3, GRHL2, HGF, LHFPL5, LOXHD1, LRTOMT, MARVELD2, MIR96, MYH14, MYH9, MYO1A, PJVK, POU4F3, PRPS1, PTPRQ, SERPINB6, SIX1, SLC17A8, TPRN, or TRIOBP has been identified. In some embodiments, a subject in need of treatment is a patient in whom a mutation in MYO7A has been identified. In some embodiments, the mutation is a missense mutation. In some embodiments, the mutation is a nonsense (e.g., truncating) mutation. In some embodiments, the mutation is not silent (i.e., the mutation results in a non-wild-type amino acid at one or more positions in a polypeptide encoded from the mutated gene). In some embodiments, a subject (e.g., a human) in need of treatment is heterozygous for a MYO7A mutation. In some embodiments, a subject (e.g., a human) in need of treatment is homozygous for a MYO7A mutation. In some embodiments, a subject (e.g., a human) in need of treatment comprises two different mutant alleles of a MYO7A gene.

Aspects of the disclosure relate to methods for use with a subject, such as human or non-human primate subjects; with a host cell in situ in a subject; or with a host cell derived from a subject (e.g., ex vivo or in vitro). Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

To “treat” a disease or disorder as the term is used herein means to reduce the frequency or severity of at least one sign or symptom of a disease or disorder experienced by a subject. The compositions described herein (e.g., compositions comprising CRISPR reagents) are typically administered to a subject in an effective amount, that is, an amount capable of producing a desirable result. The desirable result will depend upon the active agent being administered. For example, an effective amount of a composition comprising a Cas endonuclease may be an amount of the composition that is capable of facilitating cleavage of a target gene in one or more cells. A therapeutically acceptable amount may be an amount that is capable of treating a disease or condition, such as a condition described herein, including a hearing loss condition. As is well known in the medical and veterinary arts, dosage for any one subject depends on many factors, including the subject's size, body surface area, age, the particular composition to be administered, the active ingredient(s) in the composition, time and route of administration, general health, and other therapeutics being administered concurrently. For example, a therapeutically acceptable amount or effective amount of a composition disclosed herein may comprise 0.5 mg/kg to 50 mg/kg of gRNA, 1 mg/kg to 250 mg/kg of a Cas endonuclease (e.g., Cas9), and/or 0.5 mg/kg to 50 mg/kg of template nucleic acid (e.g., an HDR template oligonucleotide).

Methods disclosed herein in some embodiments comprise administration to a subject of a composition (e.g., a Cas endonuclease, a template nucleic acid, a gRNA, or a combination thereof, or an extracellular vesicle comprising one or more compounds). Compositions disclosed herein can be administered to a subject in a manner that is pharmacologically useful. In some embodiments, compositions disclosed herein are pharmaceutically acceptable compositions. In some embodiments, compositions disclosed herein are administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intratympanically, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a composition disclosed herein is administered to the subject by injection into or near the car. In some embodiments, a composition disclosed herein is administered directly to the inner ear of a subject. In some embodiments, a composition disclosed herein is administered via intratympanic injection. In some embodiments, a composition disclosed herein is administered via ear drops. In some embodiments, the subject to whom the composition is administered is a human subject.

“Treatment” of a disease, disorder or condition does not require curing the disease, disorder or condition. As used herein, treatment of a disease (e.g., a hearing loss disease) does not require complete alleviation of a symptom or symptoms of the disease in a subject to whom treatment is administered. For example, treatment of a hearing loss disease does not require full restoration of hearing in a treated subject. Treatment in some embodiments involves improvement in hearing loss in a treated subject, reduction in severity of hearing loss in a subject, improvement in the ability of a subject to detect or perceive sound, or partial mitigation of a symptom of hearing loss in a treated subject.

### EXAMPLES

The following examples are included to demonstrate illustrative embodiments of the invention and are not considered limiting. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

Described here are strategies for using CRISPR/Cas9 to facilitate correction of a missense mutation in MYO7A associated with SNHL, such as by delivery of Cas9/gRNA RNP complexes and template nucleic acids to hair cells. Such delivery can be achieved by encapsulating RNP complexes and template nucleic acids within extracellular vesicles or exosomes. EVs/exosomes derived from hair cells and hair-like cells, such as HEI-OC1 cells. These strategies are also adaptable to other “deaf” mutations in genes associated with hearing. The gene therapy function is validated by sequencing assessment of editing efficiency including knock-out and knock-in yield from delivering genome editing reagents to primary fibroblast cells dissociated from ear tissues of Shaker-1 mice, representing a MYO7A-mutant in vitro cellular model. Shaker-1 mice are a pre-clinical animal model of myosin VIIa deafness.

This example uses CRISPR/Cas9 technology to target mutated MYO7A gene containing a G to C mutation associated which results in an arginine to proline amino acid alteration. The methods described enable correction of the mutation by MYO7A cleavage and HDR based on a single stranded DNA donor template. The Cas9/gRNA complex and DNA template are designed to be encapsulated in exosomes for targeted delivery to inner ear hair cells, facilitating correction of the MYO7A gene mutation, leading to restoration of hearing. This represents a new strategy in gene therapy for hearing loss diseases. Also provided are the creative transfection method applicable for encapsulating genome editing complexes (synthetic or wild-type/unmodified) into biological nanovesicles. The methods described will be of great significance in therapeutic genome editing to restore sensory function of hair cells in the organ of corti.

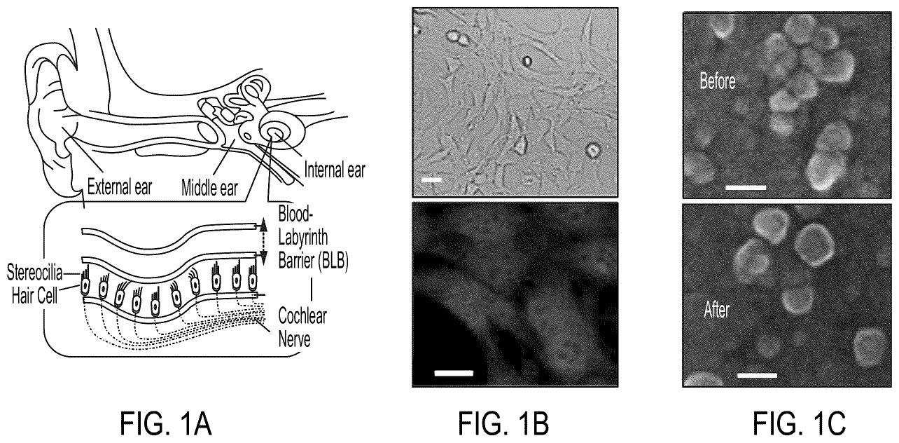

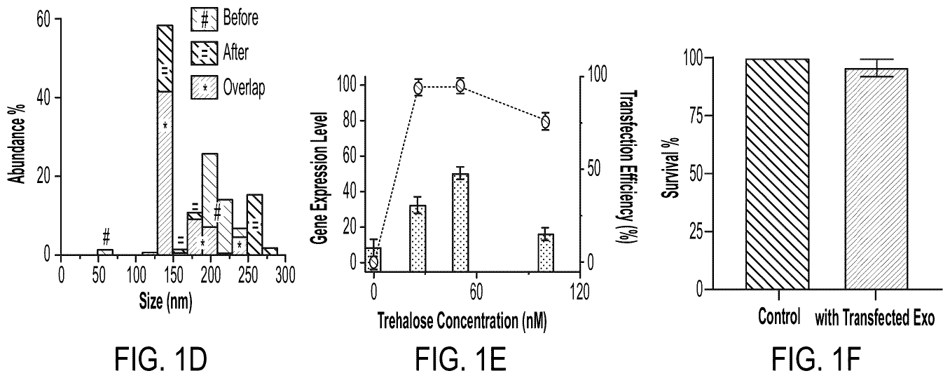

Sensorineural hearing loss (SNHL) is one of the most common neurodegenerative diseases and contributes nearly ˜90% of all hearing loss diseases [1-3], of which ˜50-60% have genetic causes [2, 4-6] with homozygous recessive mutations that induce severe hereditary hearing loss within family trees [7, 8]. The deafness resulting from genotype to phenotype expression has been well defined [4], providing a basis for developing a curable gene replacement therapy via exogenous expression of wildtype (WT) genes [9]. However, there is no efficient and targeted delivery approach available presently for delivering such specific transgene expression in vivo. Existing delivery approaches for SNHL include intratympanic injection and hydrogels to deliver drugs into the inner ear, each of which exhibit very poor therapeutic penetration through the blood-labyrinth barrier to inner ear (FIG. 1A), slow and non-specific targeting, and substantial inconsistency in drug delivery efficacy [10]. In order to overcome this clinical challenge in treating or curing SNHL, it was hypothesized that exosomes carrying CRISPR reagents (e.g., Cas9/gRNA-RNP complexes and HDR template nucleic acids) can be used to effectively cross the blood-labyrinth barrier for targeted delivery and gene therapy in the inner car.

Exosomes are membrane vesicles secreted from live cells, and have a typical size range of 30-150 nm [11, 12]. They are natural in origin with no toxicity, and have low immunogenicity in vivo [13]. Exosomes can carry important signaling biomolecules for intercellular transfer of mRNA, microRNA, and proteins such as enzymes, each of which can affect cellular function [14, 15]. Recently it has been shown that exosomes possess the ability of to cross the blood-brain barrier, a feat which is difficult or impossible for other nanoparticle or biomaterials [13, 16, 17]. The inventors of the present disclosure realized the potential for exosomes carrying CRISPR reagents to be a powerful delivery vehicle to treat or cure SNHL disease, functioning as a targeted gene-editing tool. Such engineered exosomes are capable of high loading capacity, efficient delivery, and on-target gene therapy, thereby meeting clinical needs and proving superior to current existing treatment strategies.

Based on the natural origin of exosomes for intercellular transfer of well-preserved genetic information [14], exosome-based delivery has emerged as an approach for targeted delivery to specific tissues or cell types [13, 14, 16-19]. Exosome-encapsulated drugs have proven valuable in addressing multiple clinical issues such as therapeutic resistance and toxicity to the blood-brain barrier [14]. However, efficient cargo loading to produce viable exosome delivery vehicles is still very challenging for translation into clinical utility, due to exosomes' complicated molecular components and heterogeneous subtypes from exosome processing.

According to the present disclosure, exosomes derived from HEI-OC1 cells, common progenitor cells for hair and supporting cells in the organ of corti, can be used to deliver Cas9/gRNA ribonucleoprotein (RNP) complexes to correct a mutation in MYO7A. Such HEI-OC1 cell-derived exosomes are naturally presented between the blood-labyrinth barrier in the inner car for cellular regulation (see FIG. 1A). Thus, the Cas9/gRNA RNP complex-loaded HEI-OC1 exosomes are capable of crossing the inner car blood labyrinth barrier in vivo to specifically target and correct a mutation in MYO7A. After transfection, Cas9/gRNA RNP complexes can be detected at a high level [21-23] within a shorter time of enzymatic action and achieve precise control over activity [24, 25]. Most importantly, delivery of RNP complexes does not involve the use of DNA, plasmid or viral delivery, and therefore no unwanted DNA footprints are left in the host genome [24, 26, 27], thereby conferring higher safety and specificity than previous gene therapy techniques. The use of additional reagents such as trehalose can preserve exosomes with superior stability and less membrane fusion and leakage following electroporation-mediated transfection, providing utility in clinical settings (FIGS. 1A-1F).

HEI-OC1 cells were cultured, and exosomes were collected, which demonstrated high quality (FIG. 1B). Benchtop electroporation-mediated transfection of the exosomes was conducted. The electroporation protocols provided herein preserve the morphology and size of transfected exosomes after electric pulsing (FIGS. 1C and 1D). A chemical coating reagent, trehalose, was introduced during electro-transfection, which resulted in enhanced exosome stability with less membrane fusion and leakage, in turn, improving the electroporation-mediated transfection efficiency and gene expression level provided by exosome delivery (FIG. 1E). The trehalose-treated electroporated exosomes demonstrated high biocompatibility (FIG. 1F).

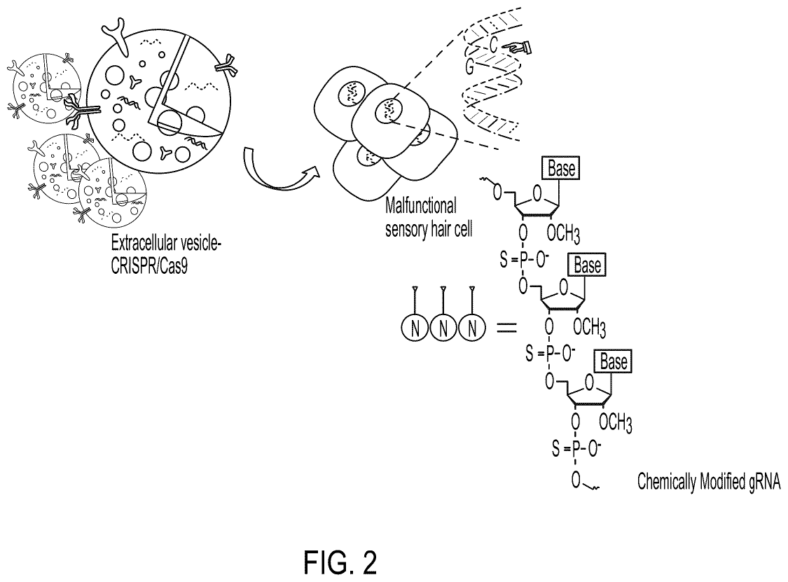

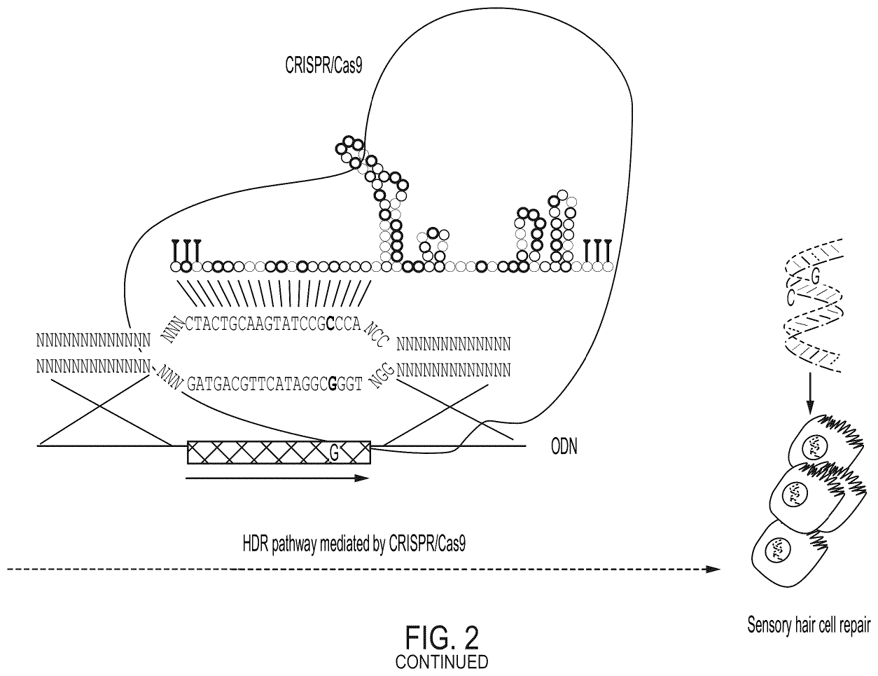

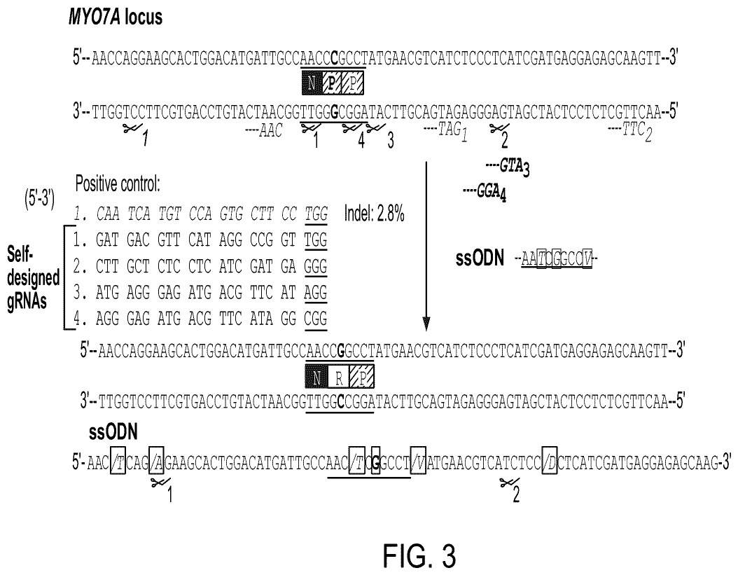

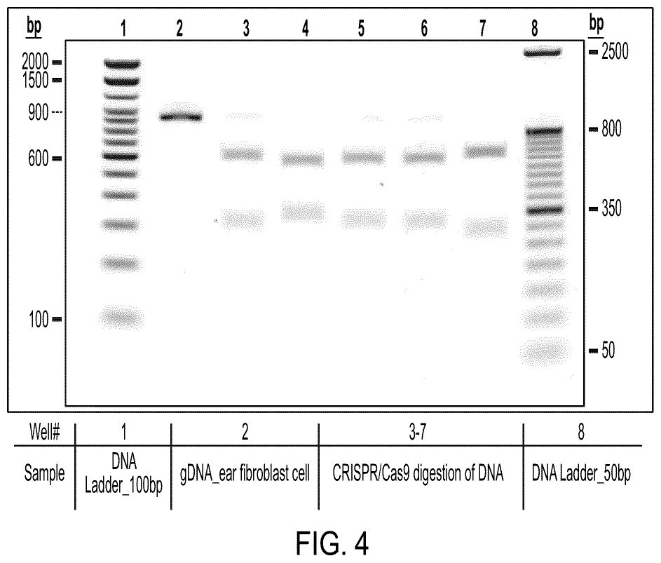

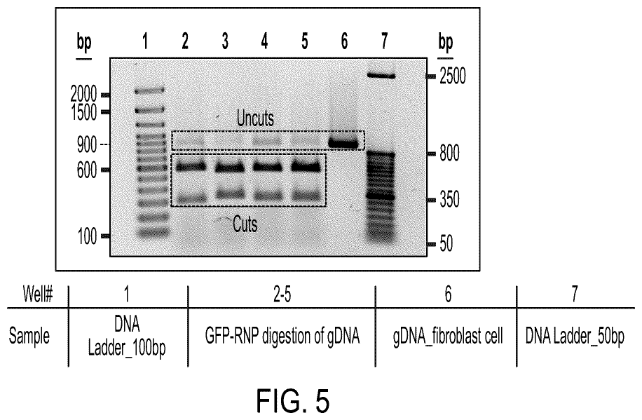

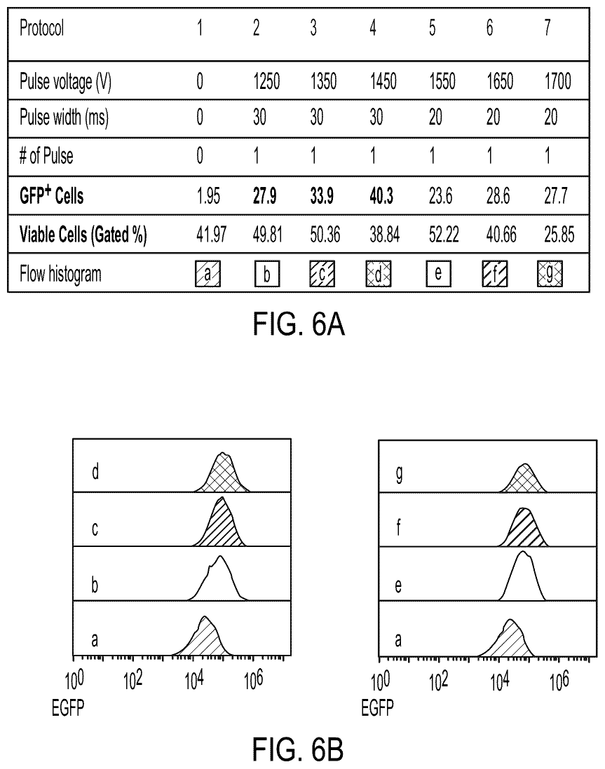

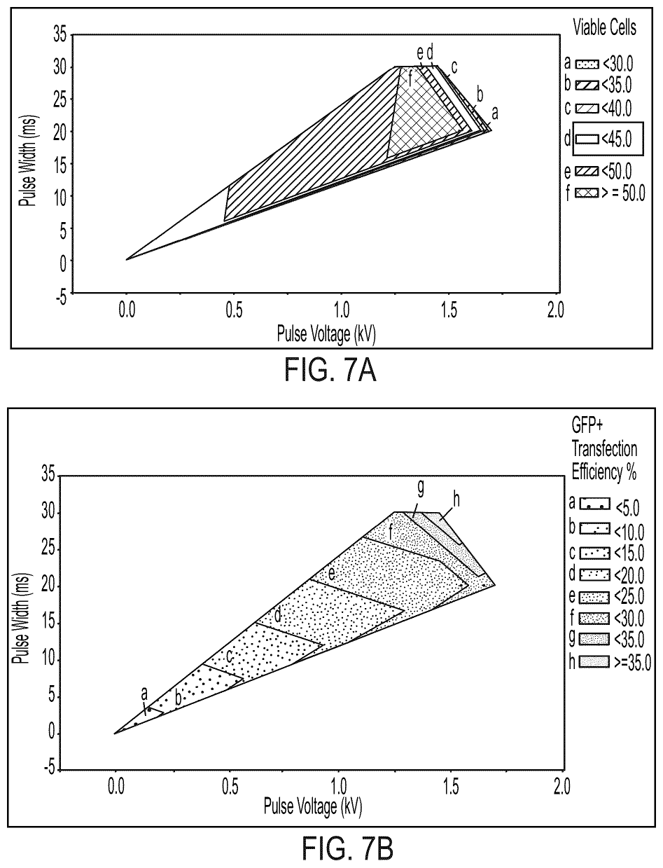

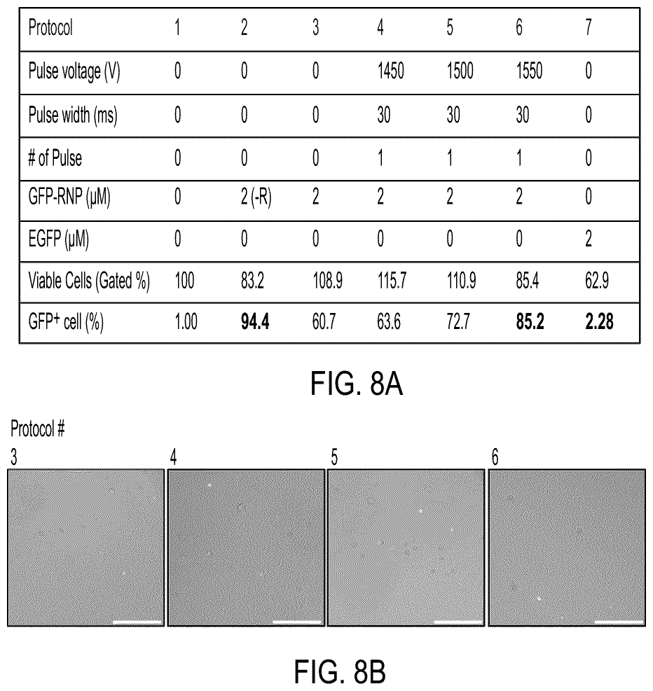

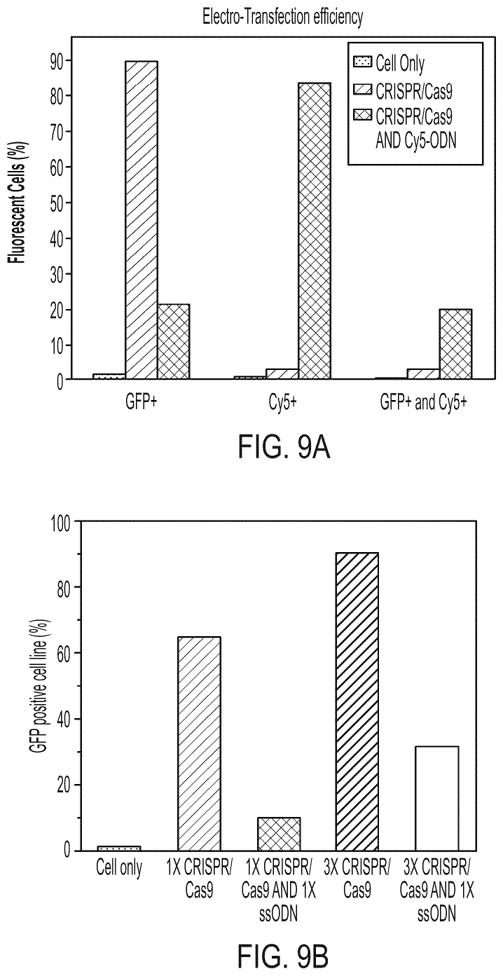

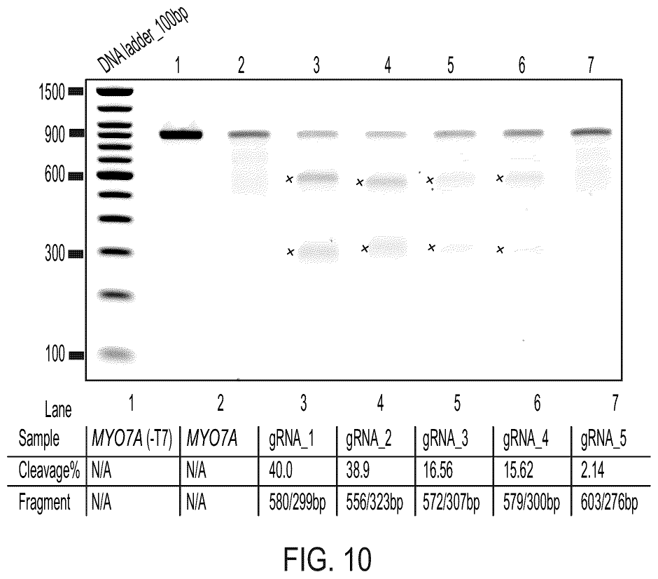

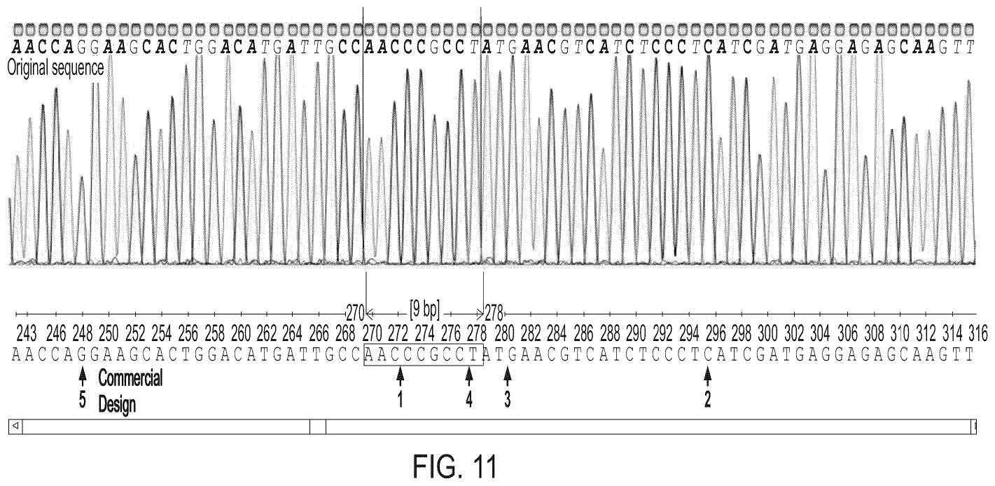

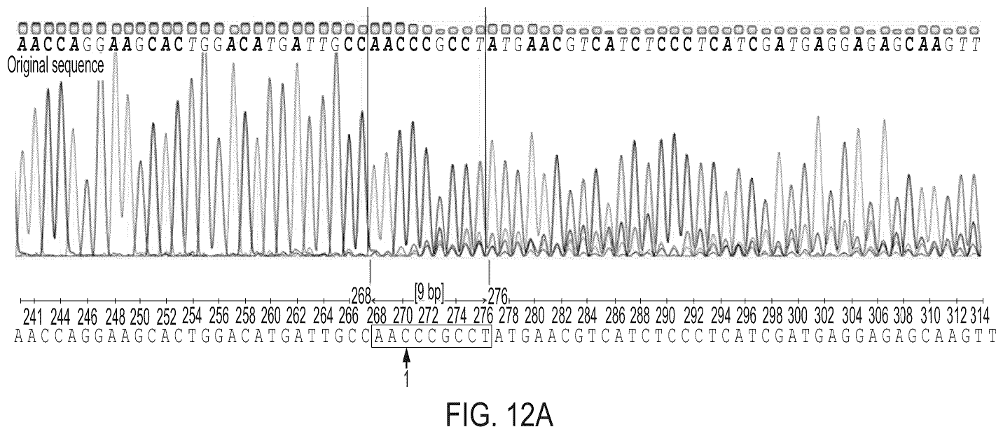

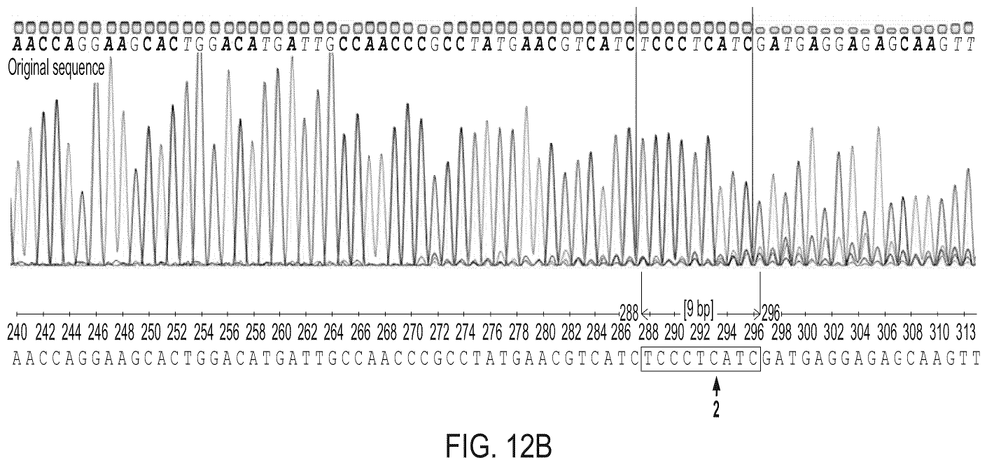

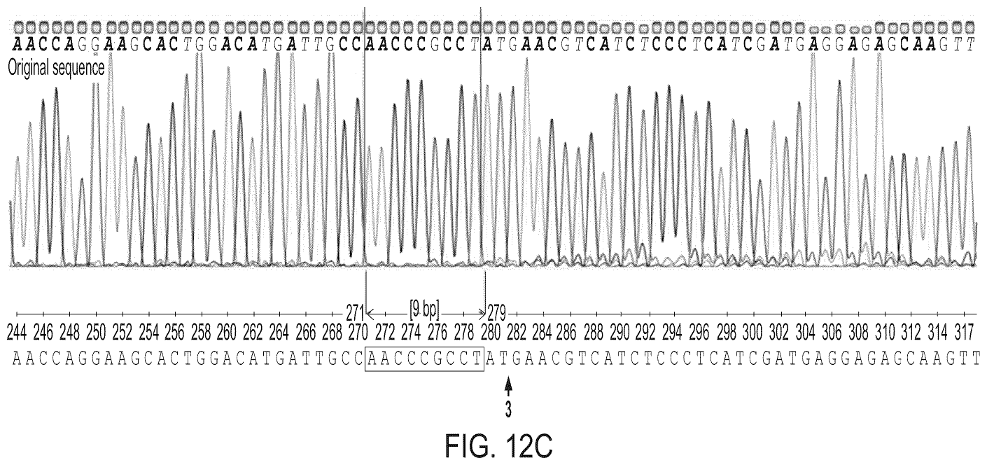

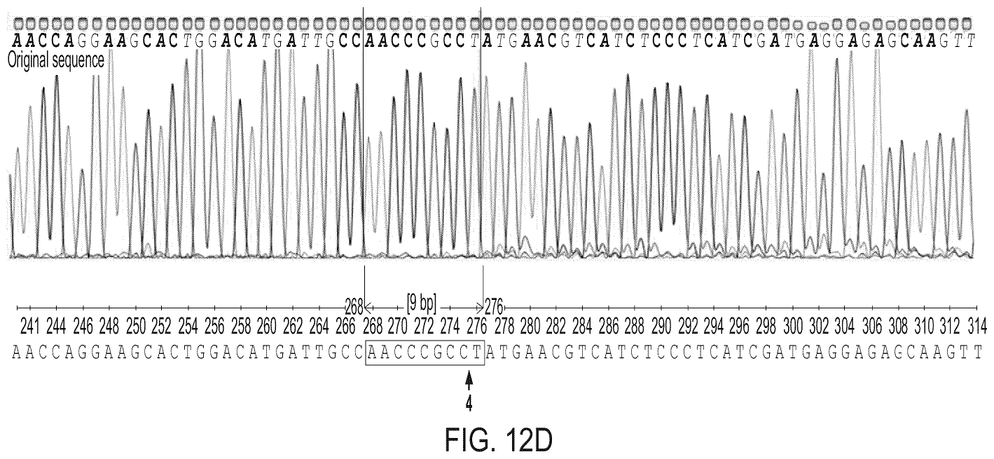

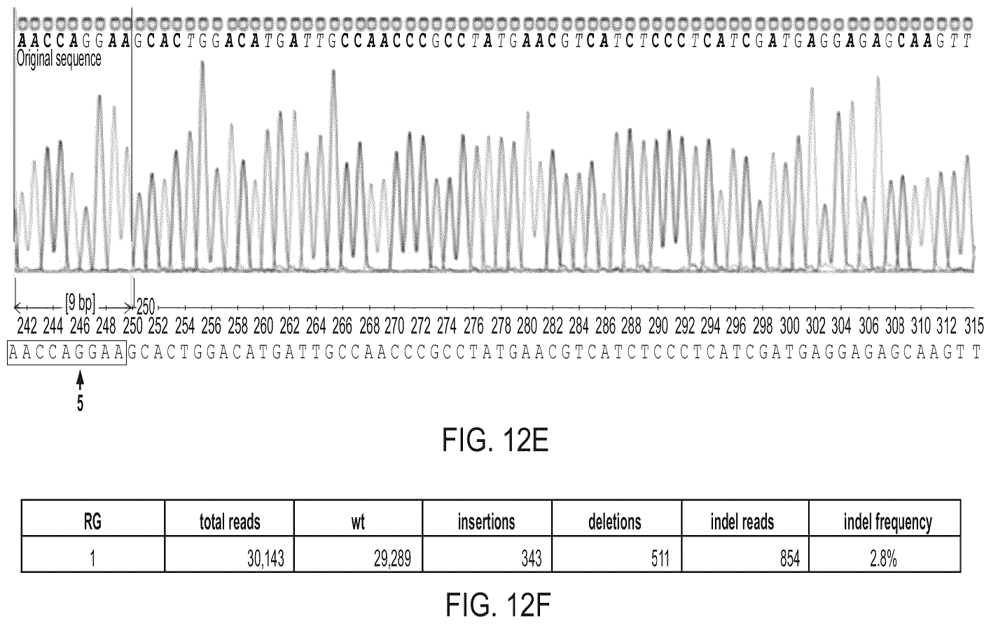

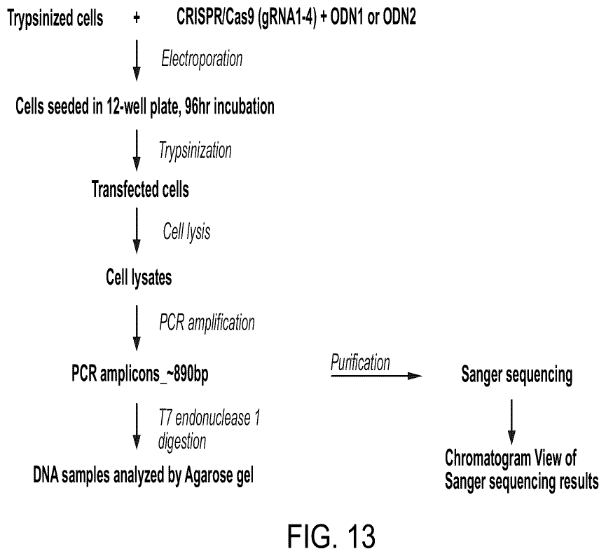

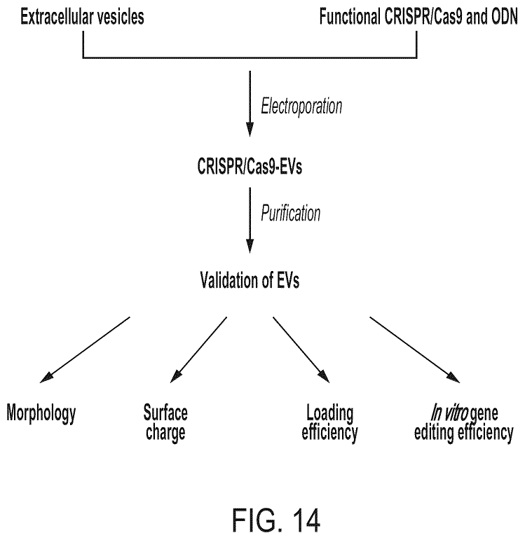

The Cas9/gRNA RNP complex and donor template nucleic acid can be used in the exosome gene therapy system disclosed to correct a MYO7A mutation. This concept is illustrated in FIG. 2, and CRISPR construct design and gene editing validation are demonstrated in FIGS. 3-4. The schematic illustrated in FIG. 2 shows gene correction (e.g., facilitated by HDR), but it should be appreciated that similar methods resulting in gene knockout (e.g., by delivering gRNA/Cas9 RNP complexes without an HDR template oligonucleotide, such that insertions or deletions are introduced into the target locus). FIGS. 5, 6A-6B, 7A-7B, 8A-8B, 9A-9B, 10, 11, and 12A-12F demonstrate validation of the gRNA designs 1, 2, 3, and 4 in a CRISPR system with Cas9 to effectively cleave MYO7A gene from primary fibroblast car cells. In contrast, a commercially designed gRNA (gRNA5) was unable to achieve effective gene cleavage under the same in vitro conditions. These results demonstrate that the CRISPR system design and construction are effective at facilitating cleavage of MYO7A in a primary cell model. FIG. 13 shows the workflow for testing Cas9/gRNA complexes and HDR template nucleic acid sequences. FIG. 14 shows workflows for testing CRISPR systems encapsulated within extracellular vesicles/exosomes. Such extracellular vesicles can be used to deliver CRISPR systems into hair cells in vitro and in vivo for correction of gene mutations.

### REFERENCES FOR EXAMPLE 1

1. Gao, X., et al., Treatment of autosomal dominant hearing loss by in vivo delivery of genome editing agents. Nature, 2018. 553(7687): p. 217-221.
2. Zhang, W., et al., Cochlear Gene Therapy for Sensorineural Hearing Loss: Current Status and Major Remaining Hurdles for Translational Success. Front Mol Neurosci, 2018. 11: p. 221.
3. Müller, U. and P. G. Barr-Gillespie, New treatment options for hearing loss. Nature reviews Drug discovery, 2015. 14(5): p. 346-365.
4. Smith, R. J., J. F. Bale Jr, and K. R. White, Sensorineural hearing loss in children. The Lancet, 2005. 365(9462): p. 879-890.
5. Parker, M. and M. Bitner Glindzicz, Genetic investigations in childhood deafness. Archives of disease in childhood, 2015. 100(3): p. 271-278.
6. Landegger, L. D., et al., A synthetic AAV vector enables safe and efficient gene transfer to the mammalian inner ear. Nature biotechnology, 2017. 35(3): p. 280.
7. Lenz, D. R. and K. B. Avraham, Hereditary hearing loss: from human mutation to mechanism. Hearing research, 2011. 281(1-2): p. 3-10.
8. Shearer, A. E., et al., Advancing genetic testing for deafness with genomic technology. Journal of medical genetics, 2013. 50(9): p. 627-634.
9. Sacheli, R., et al., Gene transfer in inner ear cells: a challenging race. Gene therapy, 2013. 20(3): p. 237.
10. Li, L., et al., Advances in nano-based inner ear delivery systems for the treatment of sensorineural hearing loss. Adv Drug Deliv Rev, 2017. 108: p. 2-12.
11. Wong, E. H. C., et al., Inner ear exosomes and their potential use as biomarkers. PLOS One, 2018. 13(6): p. e0198029.
12. Hong, C. S., et al., Isolation of biologically active and morphologically intact exosomes from plasma of patients with cancer. Journal of extracellular vesicles, 2016. 5(1): p. 29289.
13. Das, C. K., et al., Exosome as a Novel Shuttle for Delivery of Therapeutics across Biological Barriers. Mol Pharm, 2018.
14. Zhu, Q. F., et al., Microfluidic engineering of exosomes: editing cellular messages for precision therapeutics. Lab on a Chip, 2018. 18(12): p. 1690-1703.
15. Zhang, H. G. and W. E. Grizzle, Exosomes: a novel pathway of local and distant intercellular communication that facilitates the growth and metastasis of neoplastic lesions. The American journal of pathology, 2014. 184(1): p. 28-41.
16. Alvarez Erviti, L., et al., Delivery of siRNA to the mouse brain by systemic injection of targeted exosomes. Nat Biotechnol, 2011. 29(4): p. 341-5.
17. El Andaloussi, S., et al., Exosomes for targeted siRNA delivery across biological barriers. Adv Drug Deliv Rev, 2013. 65(3): p. 391-7.
18. Cooper, J. M., et al., Systemic exosomal siRNA delivery reduced alpha-synuclein aggregates in brains of transgenic mice. Mov Disord, 2014. 29(12): p. 1476-85.
19. El-Andaloussi, S., et al., Exosome-mediated delivery of siRNA in vitro and in vivo. Nat Protoc, 2012. 7(12): p. 2112-26.
20. Gyorgy, B., et al., Rescue of Hearing by Gene Delivery to Inner-Ear Hair Cells Using Exosome-Associated AAV. Molecular Therapy, 2017. 25(2): p. 379-391.
21. Rupp, L. J., et al., CRISPR/Cas9-mediated PD-1 disruption enhances anti-tumor efficacy of human chimeric antigen receptor T cells. Scientific reports, 2017. 7(1): p. 737.
22. Hendel, A., et al., Chemically modified guide RNAs enhance CRISPR-Cas genome editing in human primary cells. Nature biotechnology, 2015. 33(9): p. nbt. 3290.
23. Schumann, K., et al., Generation of knock-in primary human T cells using Cas9 ribonucleoproteins. Proceedings of the National Academy of Sciences, 2015. 112(33): p. 10437-10442.
24. Woo, J. W., et al., DNA-free genome editing in plants with preassembled CRISPR-Cas9 ribonucleoproteins. Nature biotechnology, 2015. 33(11): p. 1162.
25. Paix, A., et al., High efficiency, homology-directed genome editing in Caenorhabditis elegans using CRISPR-Cas9 ribonucleoprotein complexes. Genetics, 2015. 201(1): p. 47-54.
26. Liang, Z., et al., Efficient DNA-free genome editing of bread wheat using CRISPR/Cas9 ribonucleoprotein complexes. Nature communications, 2017. 8: p. 14261.
27. Seki, A. and S. Rutz, Optimized RNP transfection for highly efficient CRISPR/Cas9-mediated gene knockout in primary T cells. Journal of Experimental Medicine, 2018. 215(3): p. 985-997.

### Example 2

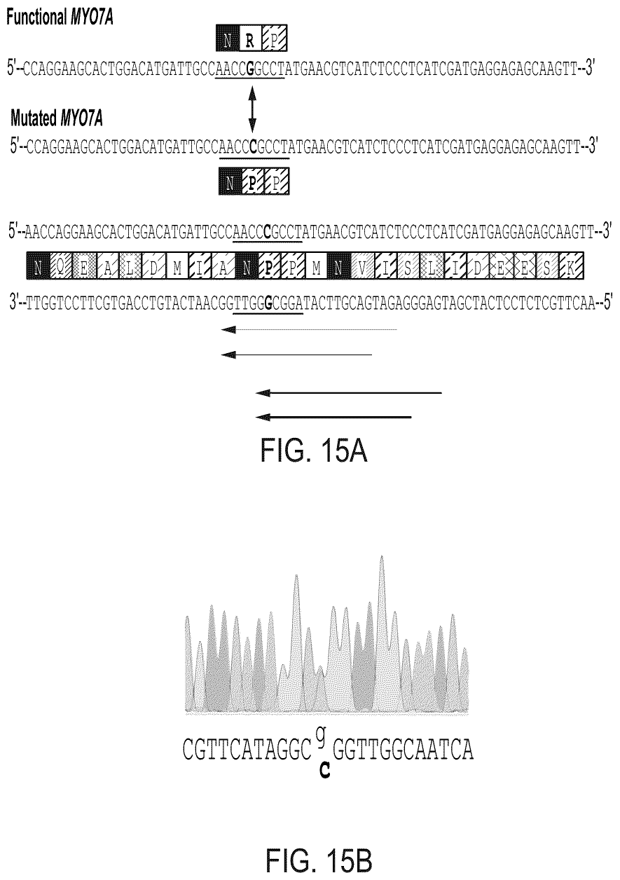

Mutations in Myo7a represent an opportunity to use CRISPR technology to treat SNHL. As shown in FIG. 15A, a single point mutation in Myo7a (G1601C) results in a single amino acid substitution (R502P), which is a common cause of SNHL. To this end, gRNAs were designed to knockout the Myo7a^{sh1} single mutation to halt the progressive hearing loss observed in the heterozygous Shaker-1 mouse model. Heterozygous or homozygous Shaker-1 mice, a pre-clinical animal model of myosin VIIa deafness, provide an opportunity to study the effects of gene editing on mutant Myo7a. FIG. 15B shows Sanger sequencing traces of Myo7a from heterozygous (Myo7a^{WT/sh1}) Shaker-1 mice, showing both the wild-type (with a G at position 1601) and mutant (C at 1601) allele sequences.

In this Example, various guide RNAs (gRNAs) were developed and tested for their ability to induce gene editing when used in combination with Cas9 endonuclease in CRISPR constructs. The sequences used in this Example are provided in Table 1 below.

TABLE 1
Sequences

| Name | Sequence (5′-3′) | SEQ ID NO: |
|---|---|---|
| gRNA-1 | GAUGACGUUCAUAGGCGGGU | 40 |
| Tru-gRNA-1 | GACGUUCAUAGGCGGGU | 41 |
| gRNA-2 | AGGGAGAUGACGUUCAUAGG | 42 |
| Tru-gRNA-2 | GAGAUGACGUUCAUAGG | 43 |
| gRNA-2_KI | CUUGCUCUCCUCAUCGAUGA | 44 |
| gRNA-3_KI | AUGAGGGAGAUGACGUUCAU | 45 |
| gRNA-4_KI | AGGGAGAUGACGUUCAUAGG | 42 |
| Forward Primer_Sanger | GAGGGAACAGAGTGGCTATTAC | 31 |
| sequencing | | |
| Reverse Primer_Sanger | GCGTAGGAGTTGGACTTGATAG | 32 |
| sequencing | | |
| Forward Primer_NGS* | ACACTCTTTCCCTACACGACGCTCTTCCGATCTC | 53 |
| | CCAGGTCAAGCCAATTCTAT | |
| Reverse Primer_NGS* | GACTGGAGTTCAGACGTGTGCTCTTCCGATCTCT | 54 |
| | TCGAGCAGCTCTGCATTA | |
| ODN-1 HDR template | TGAGTTCACTGACAACCAGGAAGCACTGGACATG | 51 |
| | ATTGCCAATCGGCCAATGAACGTCATCTCGCTCA | |
| | TCGATGAGGAGAGCAAGTTCCCCAAGGT | |
| ODN-2 HDR template | TTGCTGTGCCCACCTTGGGGAACTTGCTCTCTTC | 52 |
| | ATCAATGAGTGAGATGACGTTCATAGGCCGGITG | |
| | GCAATCATGTCCAGTGCTTCCTGGTTGT | |

*The underlined nucleotides are adapters for use in next-generation sequencing (Illumina).

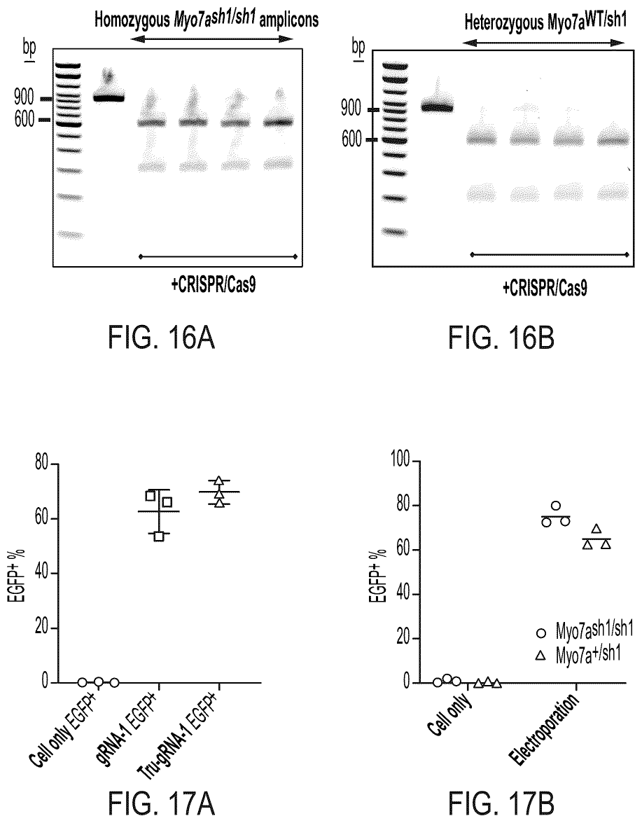

To test the gene editing activity of various gRNAs, a cell-free bioactivity assay was conducted. Myo7a amplicons were amplified from homozygous Myo7a^{sh1/sh1} mouse samples and heterozygous Myo7a^{WT/sh1} mouse samples, and subsequently treated with Cas9/gRNA ribonucleoprotein (RNP) complexes, prepared with gRNA-1, Tru-gRNA-1, gRNA-2, or Tru-gRNA-2. The resulting samples were analyzed by agarose gel electrophoresis. The results shown in FIGS. 16A and 16B show that the assembled RNP complexes have high targeting and cleavage abilities when incubated with Myo7a amplicons in cell-free conditions.

To characterize the efficiency of electroporation transfection of fibroblasts with gRNA/Cas9 RNP complexes, Cas9 labeled with EGFP was used to generate fluorescent RNP complexes. Following in vitro electroporation transfection of homozygous Myo7a^{sh1/sh1} fibroblast cells with gRNA-1/EGFP-Cas9 or Tru-gRNA-1/EGFP-Cas9 RNP complexes, cells were analyzed by flow cytometry to measure EGFP signal. The results presented in FIG. 17A shows that RNP complexes prepared with both gRNA-1 and Tru-gRNA-1 efficiently transfected homozygous Myo7a^{sh1/sh1} fibroblasts. In addition, following in vitro electroporation transfection of both homozygous Myo7a^{sh1/sh1} and heterozygous Myo7a^{WT/sh1} fibroblast cells with EGFP-Cas9/gRNA-1 RNP complexes, cells were analyzed by flow cytometry to measure EGFP signal. The results presented in FIG. 17B show that both Myo7a^{sh1/sh1} and Myo7a^{WT/sh1} fibroblasts were efficiently transfected by electroporation with the RNP complexes.

To evaluate the in vitro editing efficiency of various guide RNAs in RNP complexes, Myo7a amplicons from fibroblast cells of homozygous mutant Myo7a^{sh1/sh1}, heterozygous Myo7a^{WT/sh1}, and homozygous wild-type Myo7a^{WT/WT} mice were tested with different guide RNAs. Myo7a amplicons were incubated with RNP complexes containing Cas9 and gRNA-1, gRNA-2, Tru-gRNA-1, or Tru-gRNA-2, and treated with T7E1. Samples were subsequently subjected to agarose gel electrophoresis to determine the extent of gene editing in each sample type and facilitated by each gRNA. Percent cleavage of each sample type and facilitated by each gRNA are shown in Table 1 below. Cleavage % was calculated according to the formula: % cleavage=(1−(1−fraction cleaved)^{1/2})*100.

TABLE 2

| | Cleavage % | | | | |
|---|---|---|---|---|---|
| | Negative | | Tru- | | Tru- |
| Group | control | gRNA-1 | gRNA-1 | gRNA-2 | gRNA-2 |
| Myo7a^{sh1/sh1} | 0.0 | 44.4 | 9.5 | 41.7 | 27.9 |
| Myo7a^{WT/sh1} | 4.1 | 23.8 | 14.2 | 23.3 | 17.8 |
| Myo7a^{WT/WT} | 0.0 | 0.0 | 0.0 | 0.0 | 0.0 |

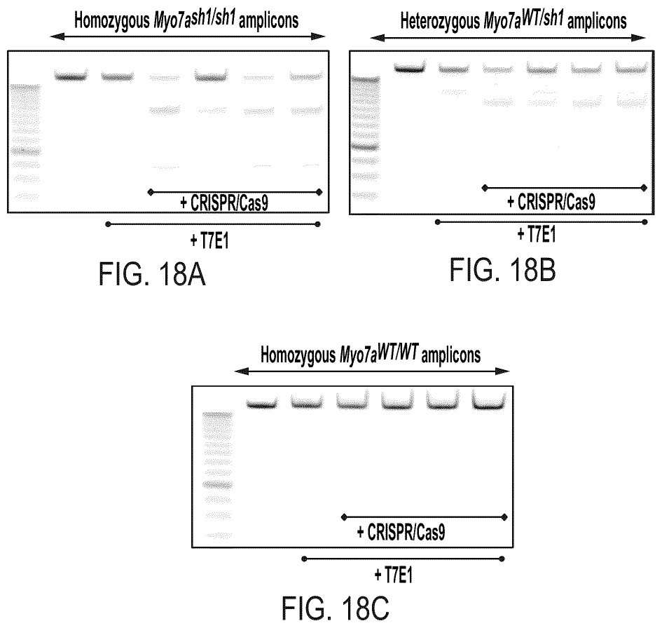

The results shown in FIGS. 18A, 18B, and 18C and Table 2 demonstrate that the CRISPR systems tested have good editing ability against Myo7a^{sh1} mutants and little or no editing activity against Myo7a^{WT}.

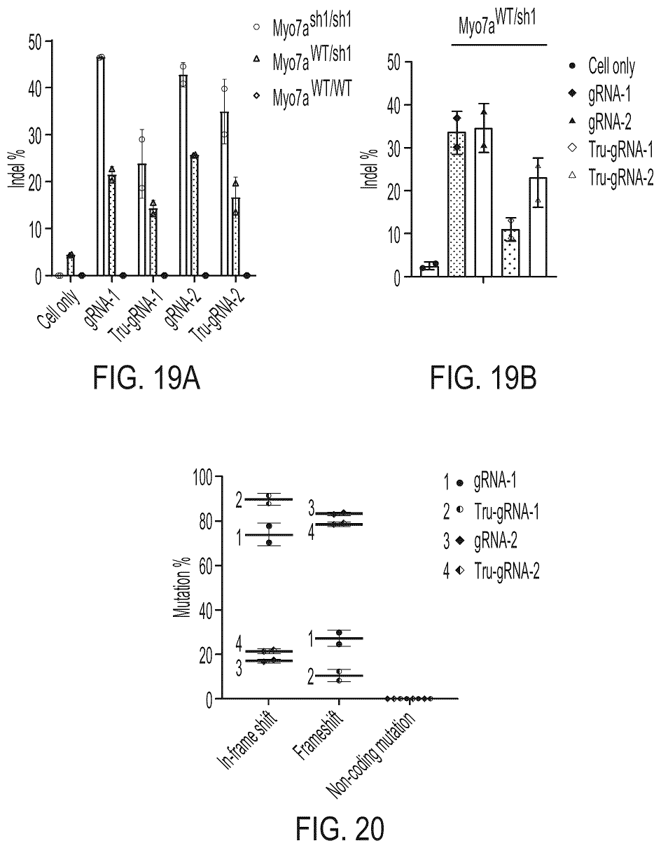

Next, gene editing efficiency facilitated by various guide RNAs was tested by quantifying the percentage of gene copies carrying insertions and/or deletions (“indels”). Homozygous mutant Myo7a^{sh1/sh1} fibroblast cells, heterozygous Myo7a^{WT/sh1} fibroblast cells, and homozygous wild-type Myo7a^{WT/WT} cells were transfected by electroporation with Cas9 RNP complexes produced with gRNA-1, gRNA-2, Tru-gRNA-1, or Tru-gRNA-2 and Myo7a amplicons were subsequently treated with T7E1 and subjected to agarose gel electrophoresis. Heterozygous Myo7a^{WT/sh1} cells were also transfected by electroporation with Cas9 RNP complexes produced with gRNA-1, gRNA-2, Tru-gRNA-1, or Tru-gRNA-2 and indel formation was subsequently analyzed by next-generation sequencing (Illumina). The results shown in FIGS. 19A and 19B demonstrate that each of the four gRNAs facilitated good targeting and Myo7a gene editing.

The types of mutations facilitated by various guide RNAs was tested by next-generation sequencing (Illumina) analyzed using CRISPResso2 (Clement, et al., “CRISPResso2 provides accurate and rapid genome editing sequence analysis” Nat. Biotechnol. 2019 March; 37(3):224-26; doi: 10.1038/s41587-019-0032-3). Heterozygous Myo7a^{WT/sh1} fibroblast cells were treated with Cas9 RNP complexes produced with gRNA-1, gRNA-2, Tru-gRNA-1, or Tru-gRNA-2 and Myo7a sequences were analyzed for the types of mutations present: in-frame shifts, frameshifts, and non-coding mutations. The results shown in FIG. 20 demonstrate that different gRNA designs facilitate different mutations, either in-frame shifts that lead to a certain number of amino acid substitutions in the encoded Myo7a protein or frameshift mutations that result in a completely altered amino acid sequence in the encoded Myo7a protein.

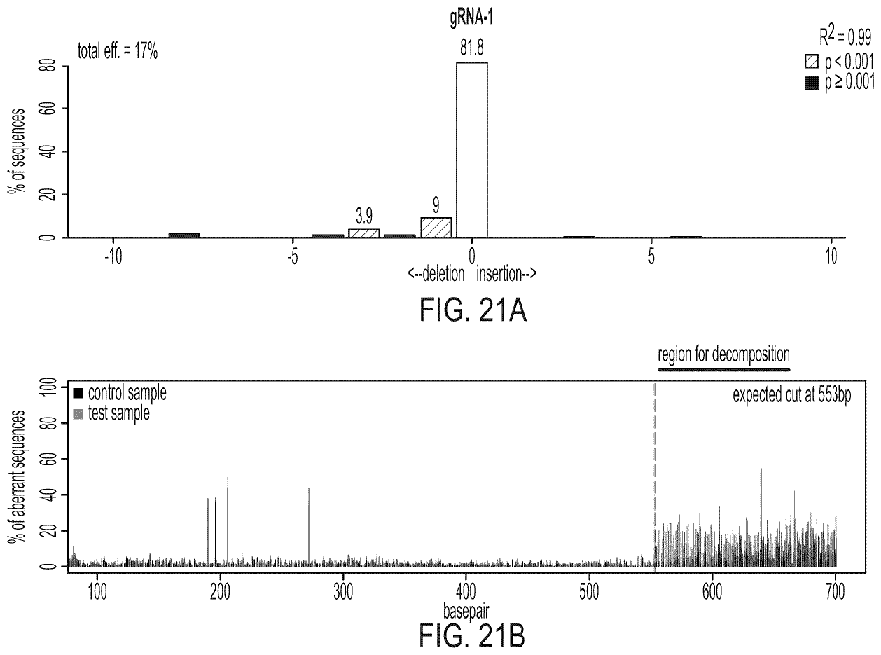

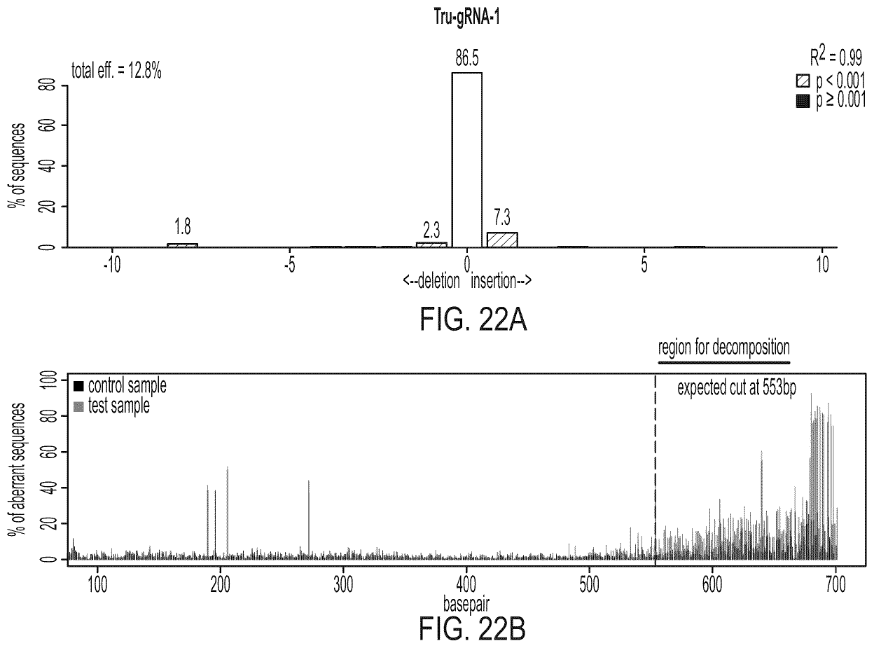

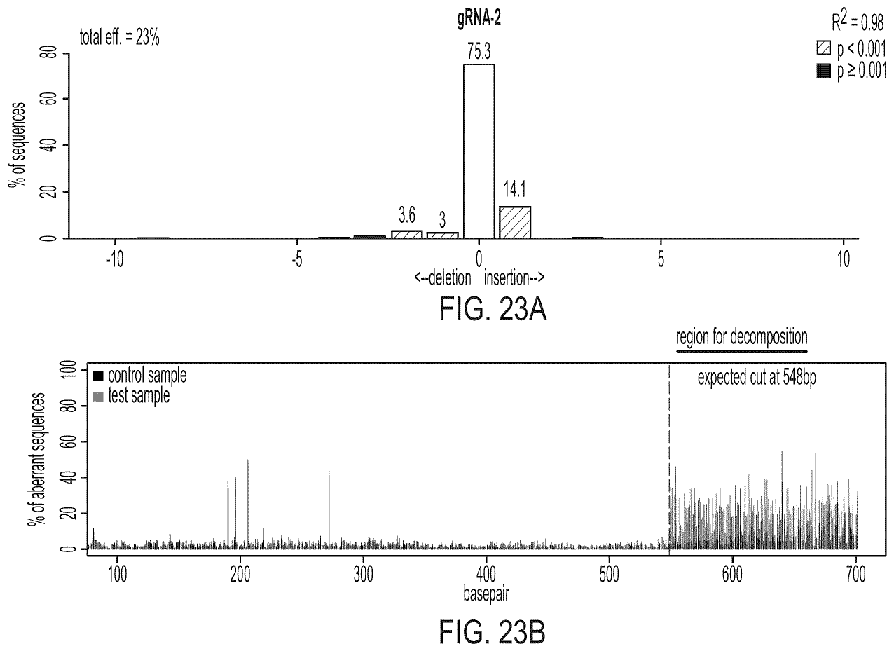

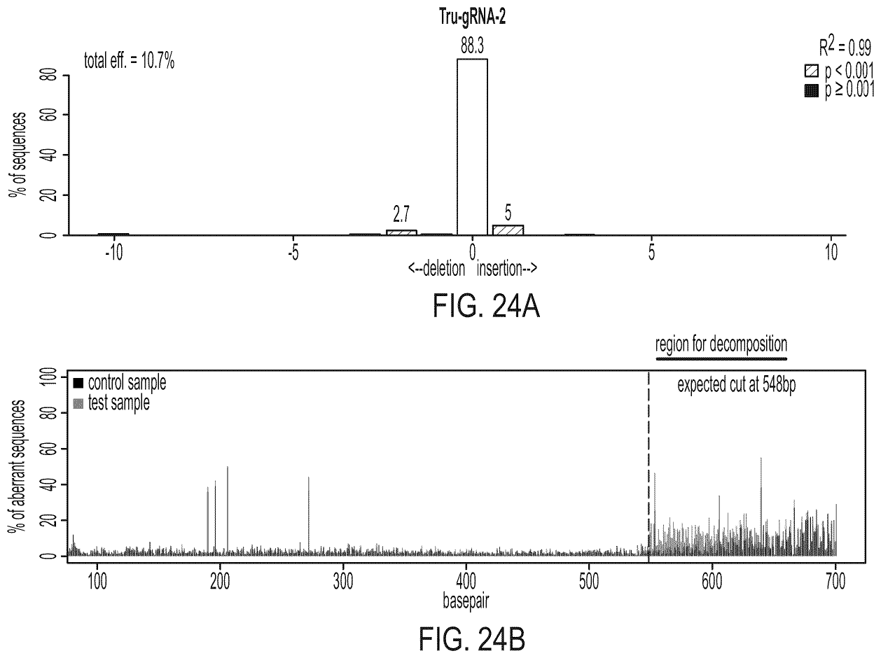

To further characterize the gene editing facilitated by different guide RNAs, tracking of indels by decomposition (“TIDE”) analysis was conducted using Sanger sequencing results of Myo7a amplicons following CRISPR treatment of heterozygous Myo7a^{WT/sh1} fibroblast cells. See Brinkman, et al., “Easy quantitative assessment of genome editing by sequence trace decomposition” Nucleic Acids Research 2014; 42(22):e168; doi: 10.1093/nar/gku936, and tide.nki.nl. TIDE analysis was conducted following treatment of cells with Cas9 RNP complexes produced with gRNA-1, gRNA-2. Tru-gRNA-1, or Tru-gRNA-2. The results shown in FIGS. 21A, 21B, 22A, 22B, 23A, 23B, 24A, and 24B demonstrate that each of the guide RNAs tested facilitated good targeting specificity and gene cleavage in vitro.

Subsequent experiments evaluated the specific capability to knock-in gene corrections using gRNAs. Using gRNA-2_KI, homozygous mutant Myo7a^{sh1/sh1} fibroblast cells were transfected with Cas9/gRNA-2_KI RNP complexes as well as ODN-2 HDR template. Next-generation sequencing of resulting Myo7a sequences demonstrated 36.9% indels, and an overall knock-in efficiency for gRNA-2_KI of 0.3% (with frameshift). These results suggest that the guide RNA can be optimized further.

### Example 3

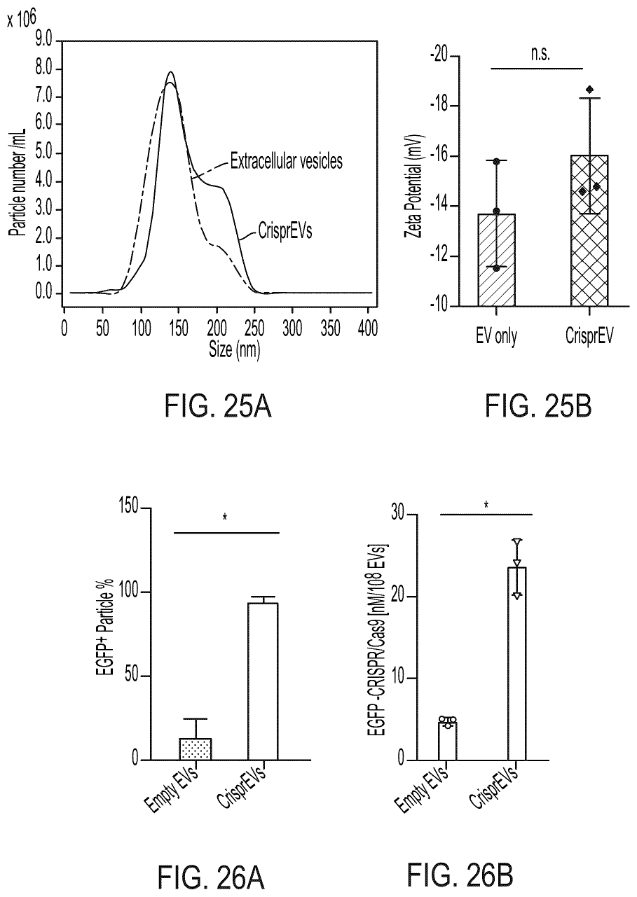

Extracellular vesicles (EVs) were loaded with CRISPR constructs and evaluated for their physical properties before and after loading. EVs were transfected with Cas9/gRNA RNP complexes (prepared with gRNA-1 or gRNA-2, as provided in Table 1) by electroporation and subsequently evaluated by nanoparticle tracking analysis (NanoSight), in comparison with EVs that were not electroporated. The results shown in FIG. 25A demonstrate that electroporation and loading of the EVs with CRISPR constructs had little effect on the size distribution of the EVs when compared with EVs that were not electroporated. EVs were further analyzed for their zeta potential (LiteSizer 500). The results shown in FIG. 25B demonstrate that the electroporation and loading of the EVs with CRISPR constructs had no significant effect on the EVs' zeta potential.

Nanoparticle tracking analysis (ZetaView) was further used to quantify the loading efficiency of EVs using EGFP-labeled Cas9. EVs were transfected with EGFP-Cas9/gRNA RNP complexes by electroporation and subsequently analyzed for EGFP fluorescence. The results shown in FIG. 26A demonstrate that greater than 90% of the EVs transfected with EGFP-Cas9/gRNA RNP complexes were positive for EGFP. The data in FIG. 26B show the amount of EGFP-Cas9 measured in 10⁸ EVs.

The results of this Example together demonstrate that CRISPR constructs were efficiently loaded into EVs by the electroporation method without affecting the physical properties of the EVs.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

SEQUENCES

Homo sapiens myosin VIIA (MYO7A), transcript variant 1, mRNA
NCBI RefSeq NM_000260.4
AGTGCTGGCTGGACAGCTGCTCTGGGCAGGAGAGAGAGGGAGAGACAAGAGACACACACAGAGAGACGGCGAGGAAG
GGAAAGACCCAGAGGGACGCCTAGAACGAGACTTGGAGCCAGACAGAGGAAGAGGGGACGTGTGTTTGCAGACTGGC
TGGGCCCGTGACCCAGCTTCCTGAGTCCTCCGTGCAGGTGGCAGCTGTACCAGGCTGGCAGGTCACTGAGAGTGGGC
AGCTGGGCCCCAGAACTGTGCCTGGCCCAGTGGGCAGCAGGAGCTCCTGACTTGGGACCATGGTGATTCTTCAGCAG
GGGGACCATGTGTGGATGGACCTGAGATTGGGGCAGGAGTTCGACGTGCCCATCGGGGCGGTGGTGAAGCTCTGCGA
CTCTGGGCAGGTCCAGGTGGTGGATGATGAAGACAATGAACACTGGATCTCTCCGCAGAACGCAACGCACATCAAGC
CTATGCACCCCACGTCGGTCCACGGCGTGGAGGACATGATCCGCCTGGGGGACCTCAACGAGGCGGGCATCTTGCGC
AACCTGCTTATCCGCTACCGGGACCACCTCATCTACACGTATACGGGCTCCATCCTGGTGGCTGTGAACCCCTACCA
GCTGCTCTCCATCTACTCGCCAGAGCACATCCGCCAGTATACCAACAAGAAGATTGGGGAGATGCCCCCCCACATCT
TTGCCATTGCTGACAACTGCTACTTCAACATGAAACGCAACAGCCGAGACCAGTGCTGCATCATCAGTGGGGAATCT
GGGGCCGGGAAGACGGAGAGCACAAAGCTGATCCTGCAGTTCCTGGCAGCCATCAGTGGGCAGCACTCGTGGATTGA
GCAGCAGGTCTTGGAGGCCACCCCCATTCTGGAAGCATTTGGGAATGCCAAGACCATCCGCAATGACAACTCAAGCC
GTTTCGGAAAGTACATCGACATCCACTTCAACAAGCGGGGCGCCATCGAGGGCGCGAAGATTGAGCAGTACCTGCTG
GAAAAGTCACGTGTCTGTCGCCAGGCCCTGGATGAAAGGAACTACCACGTGTTCTACTGCATGCTGGAGGGTATGAG
TGAGGATCAGAAGAAGAAGCTGGGCTTGGGCCAGGCCTCTGACTACAACTACTTGGCCATGGGTAACTGCATAACCT
GTGAGGGCCGGGTGGACAGCCAGGAGTACGCCAACATCCGCTCCGCCATGAAGGTGCTCATGTTCACTGACACCGAG
AACTGGGAGATCTCGAAGCTCCTGGCTGCCATCCTGCACCTGGGCAACCTGCAGTATGAGGCACGCACATTTGAAAA
CCTGGATGCCTGTGAGGTTCTCTTCTCCCCATCGCTGGCCACAGCTGCATCCCTGCTTGAGGTGAACCCCCCAGACC
TGATGAGCTGCCTGACTAGCCGCACCCTCATCACCCGCGGGGAGACGGTGTCCACCCCACTGAGCAGGGAACAGGCA
CTGGACGTGCGCGACGCCTTCGTAAAGGGGATCTACGGGCGGCTGTTCGTGTGGATTGTGGACAAGATCAACGCAGC
AATTTACAAGCCTCCCTCCCAGGATGTGAAGAACTCTCGCAGGTCCATCGGCCTCCTGGACATCTTTGGGTTTGAGA
ACTTTGCTGTGAACAGCTTTGAGCAGCTCTGCATCAACTTCGCCAATGAGCACCTGCAGCAGTTCTTTGTGCGGCAC
GTGTTCAAGCTGGAGCAGGAGGAATATGACCTGGAGAGCATTGACTGGCTGCACATCGAGTTCACTGACAACCAGGA
TGCCCTGGACATGATTGCCAACAAGCCCATGAACATCATCTCCCTCATCGATGAGGAGAGCAAGTTCCCCAAGGGCA
CAGACACCACCATGTTACACAAGCTGAACTCCCAGCACAAGCTCAACGCCAACTACATCCCCCCCAAGAACAACCAT
GAGACCCAGTTTGGCATCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAGATCTTCCAGGCCGATG
TCGCCATGGGCGCCGAGACCAGGAAGCGCTCGCCCACACTTAGCAGCCAGTTCAAGCGGTCACTGGAGCTGCTGATG
CGCACGCTGGGTGCCTGCCAGCCCTTCTTTGTGCGATGCATCAAGCCCAATGAGTTCAAGAAGCCCATGCTGTTCGA
CCGGCACCTGTGCGTGCGCCAGCTGCGGTACTCAGGAATGATGGAGACCATCCGAATCCGCCGAGCTGGCTACCCCA
TCCGCTACAGCTTCGTAGAGTTTGTGGAGCGGTACCGTGTGCTGCTGCCAGGTGTGAAGCCGGCCTACAAGCAGGGC
GACCTCCGCGGGACTTGCCAGCGCATGGCTGAGGCTGTGCTGGGCACCCACGATGACTGGCAGATAGGCAAAACCAA
GATCTTTCTGAAGGACCACCATGACATGCTGCTGGAAGTGGAGCGGGACAAAGCCATCACCGACAGAGTCATCCTCC
TTCAGAAAGTCATCCGGGGATTCAAAGACAGGTCTAACTTTCTGAAGCTGAAGAACGCTGCCACACTGATCCAGAGG
CACTGGCGGGGTCACAACTGTAGGAAGAACTACGGGCTGATGCGTCTGGGCTTCCTGCGGCTGCAGGCCCTGCACCG
CTCCCGGAAGCTGCACCAGCAGTACCGCCTGGCCCGCCAGCGCATCATCCAGTTCCAGGCCCGCTGCCGCGCCTATC
TGGTGCGCAAGGCCTTCCGCCACCGCCTCTGGGCTGTGCTCACCGTGCAGGCCTATGCCCGGGGCATGATCGCCCGC
AGGCTGCACCAACGCCTCAGGGCTGAGTATCTGTGGCGCCTCGAGGCTGAGAAAATGCGGCTGGCGGAGGAAGAGAA
GCTTCGGAAGGAGATGAGCGCCAAGAAGGCCAAGGAGGAGGCCGAGCGCAAGCATCAGGAGCGCCTGGCCCAGCTGG
CTCGTGAGGACGCTGAGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGGGACTTCAGGTGGCCT
GCCAGGCCAGGAGGGCCAGGCACCTAGTGGCTTTGAGGACCTGGAGCGAGGGCGGAGGGAGATGGTGGAGGAGGACC
TGGATGCAGCCCTGCCCCTGCCTGACGAGGATGAGGAGGACCTCTCTGAGTATAAATTTGCCAAGTTCGCGGCCACC
TACTTCCAGGGGACAACCACGCACTCCTACACCCGGCGGCCACTCAAACAGCCACTGCTCTACCATGACGACGAGGG
TGACCAGCTGGCAGCCCTGGCGGTCTGGATCACCATCCTCCGCTTCATGGGGGACCTCCCTGAGCCCAAGTACCACA
CAGCCATGAGTGATGGCAGTGAGAAGATCCCTGTGATGACCAAGATTTATGAGACCCTGGGCAAGAAGACGTACAAG
AGGGAGCTGCAGGCCCTGCAGGGCGAGGGCGAGGCCCAGCTCCCCGAGGGCCAGAAGAAGAGCAGTGTGAGGCACAA
GCTGGTGCATTTGACTCTGAAAAAGAAGTCCAAGCTCACAGAGGAGGTGACCAAGAGGCTGCATGACGGGGAGTCCA
CAGTGCAGGGCAACAGCATGCTGGAGGACCGGCCCACCTCCAACCTGGAGAAGCTGCACTTCATCATCGGCAATGGC
ATCCTGCGGCCAGCACTCCGGGACGAGATCTACTGCCAGATCAGCAAGCAGCTGACCCACAACCCCTCCAAGAGCAG
CTATGCCCGGGGCTGGATTCTCGTGTCTCTCTGCGTGGGCTGTTTCGCCCCCTCCGAGAAGTTTGTCAAGTACCTGC
GGAACTTCATCCACGGGGGCCCGCCCGGCTACGCCCCGTACTGTGAGGAGCGCCTGAGAAGGACCTTTGTCAATGGG
ACACGGACACAGCCGCCCAGCTGGCTGGAGCTGCAGGCCACCAAGTCCAAGAAGCCAATCATGTTGCCCGTGACATT
CATGGATGGGACCACCAAGACCCTGCTGACGGACTCGGCAACCACGGCCAAGGAGCTCTGCAACGCGCTGGCCGACA
AGATCTCTCTCAAGGACCGGTTCGGGTTCTCCCTCTACATTGCCCTGTTTGACAAGGTGTCCTCCCTGGGCAGCGGC
AGTGACCACGTCATGGACGCCATCTCCCAGTGCGAGCAGTACGCCAAGGAGCAGGGCGCCCAGGAGCGCAACGCCCC
CTGGAGGCTCTTCTTCCGCAAAGAGGTCTTCACGCCCTGGCACAGCCCCTCCGAGGACAACGTGGCCACCAACCTCA
TCTACCAGCAGGTGGTGCGAGGAGTCAAGTTTGGGGAGTACAGGTGTGAGAAGGAGGACGACCTGGCTGAGCTGGCC
TCCCAGCAGTACTTTGTAGACTATGGCTCTGAGATGATCCTGGAGCGCCTCCTGAACCTCGTGCCCACCTACATCCC
CGACCGCGAGATCACGCCCCTGAAGACGCTGGAGAAGTGGGCCCAGCTGGCCATCGCCGCCCACAAGAAGGGGATTT
ATGCCCAGAGGAGAACTGATGCCCAGAAGGTCAAAGAGGATGTGGTCAGTTATGCCCGCTTCAAGTGGCCCTTGCTC
TTCTCCAGGTTTTATGAAGCCTACAAATTCTCAGGCCCCAGTCTCCCCAAGAACGACGTCATCGTGGCCGTCAACTG
GACGGGTGTGTACTTTGTGGATGAGCAGGAGCAGGTACTTCTGGAGCTGTCCTTCCCAGAGATCATGGCCGTGTCCA
GCAGCAGGGAGTGCCGTGTCTGGCTCTCACTGGGCTGCTCTGATCTTGGCTGTGCTGCGCCTCACTCAGGCTGGGCA
GGACTGACCCCGGCGGGGCCCTGTTCTCCGTGTTGGTCCTGCAGGGGAGCGAAAACGACGGCCCCCAGCTTCACGCT
GGCCACCATCAAGGGGGACGAATACACCTTCACCTCCAGCAATGCTGAGGACATTCGTGACCTGGTGGTCACCTTCC
TAGAGGGGCTCCGGAAGAGATCTAAGTATGTTGTGGCCCTGCAGGATAACCCCAACCCCGCAGGCGAGGAGTCAGGC
TTCCTCAGCTTTGCCAAGGGAGACCTCATCATCCTGGACCATGACACGGGCGAGCAGGTCATGAACTCGGGCTGGGC
CAACGGCATCAATGAGAGGACCAAGCAGCGTGGGGACTTCCCCACCGACAGTGTGTACGTCATGCCCACTGTCACCA
TGCCACCGCGGGAGATTGTGGCCCTGGTCACCATGACTCCCGATCAGAGGCAGGACGTTGTCCGGCTCTTGCAGCTG
CGAACGGCGGAGCCCGAGGTGCGTGCCAAGCCCTACACGCTGGAGGAGTTTTCCTATGACTACTTCAGGCCCCCACC
CAAGCACACGCTGAGCCGTGTCATGGTGTCCAAGGCCCGAGGCAAGGACCGGCTGTGGAGCCACACGCGGGAACCGC
TCAAGCAGGCGCTGCTCAAGAAGCTCCTGGGCAGTGAGGAGCTCTCGCAGGAGGCCTGCCTGGCCTTCATTGCTGTG
CTCAAGTACATGGGCGACTACCCGTCCAAGAGGACACGCTCCGTCAACGAGCTCACCGACCAGATCTTTGAGGGTCC
CCTGAAAGCCGAGCCCCTGAAGGACGAGGCATATGTGCAGATCCTGAAGCAGCTGACCGACAACCACATCAGGTACA
GCGAGGAGCGGGGTTGGGAGCTGCTCTGGCTGTGCACGGGCCTTTTCCCACCCAGCAACATCCTCCTGCCCCACGTG
CAGCGCTTCCTGCAGTCCCGAAAGCACTGCCCACTCGCCATCGACTGCCTGCAACGGCTCCAGAAAGCCCTGAGAAA
CGGGTCCCGGAAGTACCCTCCGCACCTGGTGGAGGTGGAGGCCATCCAGCACAAGACCACCCAGATTTTCCACAAAG
TCTACTTCCCTGATGACACTGACGAGGCCTTCGAAGTGGAGTCCAGCACCAAGGCCAAGGACTTCTGCCAGAACATC
GCCACCAGGCTGCTCCTCAAGTCCTCAGAGGGATTCAGCCTCTTTGTCAAAATTGCAGACAAGGTCCTCAGCGTTCC
TGAGAATGACTTCTTCTTTGACTTTGTTCGACACTTGACAGACTGGATAAAGAAAGCTCGGCCCATCAAGGACGGAA
TTGTGCCCTCACTCACCTACCAGGTGTTCTTCATGAAGAAGCTGTGGACCACCACGGTGCCAGGGAAGGATCCCATG
GCCGATTCCATCTTCCACTATTACCAGGAGTTGCCCAAGTATCTCCGAGGCTACCACAAGTGCACGCGGGAGGAGGT
GCTGCAGCTGGGGGCGCTGATCTACAGGGTCAAGTTCGAGGAGGACAAGTCCTACTTCCCCAGCATCCCCAAGCTGC
TGCGGGAGCTGGTGCCCCAGGACCTTATCCGGCAGGTCTCACCTGATGACTGGAAGCGGTCCATCGTCGCCTACTTC
AACAAGCACGCAGGGAAGTCCAAGGAGGAGGCCAAGCTGGCCTTCCTGAAGCTCATCTTCAAGTGGCCCACCTTTGG
CTCAGCCTTCTTCGAGGTGAAGCAAACTACGGAGCCAAACTTCCCTGAGATCCTCCTAATTGCCATCAACAAGTATG
GGGTCAGCCTCATCGATCCCAAAACGAAGGATATCCTCACCACTCATCCCTTCACCAAGATCTCCAACTGGAGCAGC
GGCAACACCTACTTCCACATCACCATTGGGAACTTGGTGCGCGGGAGCAAACTGCTCTGCGAGACGTCACTGGGCTA
CAAGATGGATGACCTCCTGACTTCCTACATTAGCCAGATGCTCACAGCCATGAGCAAACAGCGGGGCTCCAGGAGCG
GCAAGTGAACAGTCACGGGGAGGTGCTGGTTCCATGCCTGCTCTCGAGGCAGCAGTGGGTTCAGGCCCATCAGCTAC
CCCTGCAGCTGGGGAAGACTTATGCCATCCCGGCAGCGAGGCTGGGCTGGCCAGCCACCACTGACTATACCAACTGG
GCCTCTGATGTTCTTCCAGTGAGGCATCTCTCTGGGATGCAGAACTTCCCTCCATCCACCCCTCTGGCACCTGGGTT
GGTCTAATCCTAGTTTGCTGTGGCCTTCCCGGTTGTGAGAGCCTGTGATCCTTAGATGTGTCTCCTGTTTCAGACCA
GCCCCACCATGCAACTTCCTTTGACTTTCTGTGTACCACTGGGATAGAGGAATCAAGAGGACAATCTAGCTCTCCAT
ACTTTGAACAACCAAATGTGCATTGAATACTCTGAAACCGAAGGGACTGGATCTGCAGGTGGGATGAGGGAGACAGA
CCACTTTTCTATATTGCAGTGTGAATGCTGGGCCCCTGCTCAAGTCTACCCTGATCACCTCAGGGCATAAAGCATGT
TTCATTCTCTGGCC (SEQ ID NO: 1)
Homo sapiens myosin VIIA (MYO7A), isoform 1, protein
NCBI RefSeq NP_000251.3
MVILQQGDHVWMDLRLGQEFDVPIGAVVKLCDSGQVQVVDDEDNEHWISPQNATHIKPMHPTSVHGVEDMIRLGDLN
EAGILRNLLIRYRDHLIYTYTGSILVAVNPYQLLSIYSPEHIRQYTNKKIGEMPPHIFAIADNCYFNMKRNSRDQCC
IISGESGAGKTESTKLILQFLAAISGQHSWIEQQVLEATPILEAFGNAKTIRNDNSSRFGKYIDIHFNKRGAIEGAK
IEQYLLEKSRVCRQALDERNYHVFYCMLEGMSEDQKKKLGLGQASDYNYLAMGNCITCEGRVDSQEYANIRSAMKVL
MFTDTENWEISKLLAAILHLGNLQYEARTFENLDACEVLFSPSLATAASLLEVNPPDLMSCLTSRTLITRGETVSTP
LSREQALDVRDAFVKGIYGRLFVWIVDKINAAIYKPPSQDVKNSRRSIGLLDIFGFENFAVNSFEQLCINFANEHLQ
QFFVRHVFKLEQEEYDLESIDWLHIEFTDNQDALDMIANKPMNIISLIDEESKFPKGTDTTMLHKLNSQHKLNANYI
PPKNNHETQFGINHFAGIVYYETQGFLEKNRDTLHGDIIQLVHSSRNKFIKQIFQADVAMGAETRKRSPTLSSQFKR
SLELLMRTLGACQPFFVRCIKPNEFKKPMLFDRHLCVRQLRYSGMMETIRIRRAGYPIRYSFVEFVERYRVLLPGVK
PAYKQGDLRGTCQRMAEAVLGTHDDWQIGKTKIFLKDHHDMLLEVERDKAITDRVILLQKVIRGFKDRSNFLKLKNA
ATLIQRHWRGHNCRKNYGLMRLGFLRLQALHRSRKLHQQYRLARQRIIQFQARCRAYLVRKAFRHRLWAVLTVQAYA
RGMIARRLHQRLRAEYLWRLEAEKMRLAEEEKLRKEMSAKKAKEEAERKHQERLAQLAREDAERELKEKEAARRKKE
LLEQMERARHEPVNHSDMVDKMFGFLGTSGGLPGQEGQAPSGFEDLERGRREMVEEDLDAALPLPDEDEEDLSEYKF
AKFAATYFQGTTTHSYTRRPLKQPLLYHDDEGDQLAALAVWITILRFMGDLPEPKYHTAMSDGSEKIPVMTKIYETL
GKKTYKRELQALQGEGEAQLPEGQKKSSVRHKLVHLTLKKKSKLTEEVTKRLHDGESTVQGNSMLEDRPTSNLEKLH
FIIGNGILRPALRDEIYCQISKQLTHNPSKSSYARGWILVSLCVGCFAPSEKFVKYLRNFIHGGPPGYAPYCEERLR
RTFVNGTRTQPPSWLELQATKSKKPIMLPVTFMDGTTKTLLTDSATTAKELCNALADKISLKDRFGFSLYIALFDKV
SSLGSGSDHVMDAISQCEQYAKEQGAQERNAPWRLFFRKEVFTPWHSPSEDNVATNLIYQQVVRGVKFGEYRCEKED
DLAELASQQYFVDYGSEMILERLLNLVPTYIPDREITPLKTLEKWAQLAIAAHKKGIYAQRRTDAQKVKEDVVSYAR
FKWPLLFSRFYEAYKFSGPSLPKNDVIVAVNWTGVYFVDEQEQVLLELSFPEIMAVSSSRECRVWLSLGCSDLGCAA
PHSGWAGLTPAGPCSPCWSCRGAKITAPSFTLATIKGDEYTFTSSNAEDIRDLVVTFLEGLRKRSKYVVALQDNPNP
AGEESGFLSFAKGDLIILDHDTGEQVMNSGWANGINERTKQRGDFPTDSVYVMPTVTMPPREIVALVTMTPDQRQDV
VRLLQLRTAEPEVRAKPYTLEEFSYDYFRPPPKHTLSRVMVSKARGKDRLWSHTREPLKQALLKKLLGSEELSQEAC
LAFIAVLKYMGDYPSKRTRSVNELTDQIFEGPLKAEPLKDEAYVQILKQLTDNHIRYSEERGWELLWLCTGLFPPSN
ILLPHVQRFLQSRKHCPLAIDCLQRLQKALRNGSRKYPPHLVEVEAIQHKTTQIFHKVYFPDDTDEAFEVESSTKAK
DFCQNIATRLLLKSSEGFSLFVKIADKVLSVPENDFFFDFVRHLTDWIKKARPIKDGIVPSLTYQVFFMKKLWTTTV
PGKDPMADSIFHYYQELPKYLRGYHKCTREEVLQLGALIYRVKFEEDKSYFPSIPKLLRELVPQDLIRQVSPDDWKR
SIVAYFNKHAGKSKEEAKLAFLKLIFKWPTFGSAFFEVKQTTEPNFPEILLIAINKYGVSLIDPKTKDILTTHPFTK
ISNWSSGNTYFHITIGNLVRGSKLLCETSLGYKMDDLLTSYISQMLTAMSKQRGSRSGK (SEQ ID NO: 2)
Homo sapiens myosin VIIA (MYO7A), transcript variant 2, mRNA
NCBI RefSeq NM_001127180.2
AGTGCTGGCTGGACAGCTGCTCTGGGCAGGAGAGAGAGGGAGAGACAAGAGACACACACAGAGAGACGGCGAGGAAG
GGAAAGACCCAGAGGGACGCCTAGAACGAGACTTGGAGCCAGACAGAGGAAGAGGGGACGTGTGTTTGCAGACTGGC
TGGGCCCGTGACCCAGCTTCCTGAGTCCTCCGTGCAGGTGGCAGCTGTACCAGGCTGGCAGGTCACTGAGAGTGGGC
AGCTGGGCCCCAGAACTGTGCCTGGCCCAGTGGGCAGCAGGAGCTCCTGACTTGGGACCATGGTGATTCTTCAGCAG
GGGGACCATGTGTGGATGGACCTGAGATTGGGGCAGGAGTTCGACGTGCCCATCGGGGCGGTGGTGAAGCTCTGCGA
CTCTGGGCAGGTCCAGGTGGTGGATGATGAAGACAATGAACACTGGATCTCTCCGCAGAACGCAACGCACATCAAGC
CTATGCACCCCACGTCGGTCCACGGCGTGGAGGACATGATCCGCCTGGGGGACCTCAACGAGGCGGGCATCTTGCGC
AACCTGCTTATCCGCTACCGGGACCACCTCATCTACACGTATACGGGCTCCATCCTGGTGGCTGTGAACCCCTACCA
GCTGCTCTCCATCTACTCGCCAGAGCACATCCGCCAGTATACCAACAAGAAGATTGGGGAGATGCCCCCCCACATCT
TTGCCATTGCTGACAACTGCTACTTCAACATGAAACGCAACAGCCGAGACCAGTGCTGCATCATCAGTGGGGAATCT
GGGGCCGGGAAGACGGAGAGCACAAAGCTGATCCTGCAGTTCCTGGCAGCCATCAGTGGGCAGCACTCGTGGATTGA
GCAGCAGGTCTTGGAGGCCACCCCCATTCTGGAAGCATTTGGGAATGCCAAGACCATCCGCAATGACAACTCAAGCC
GTTTCGGAAAGTACATCGACATCCACTTCAACAAGCGGGGCGCCATCGAGGGCGCGAAGATTGAGCAGTACCTGCTG
GAAAAGTCACGTGTCTGTCGCCAGGCCCTGGATGAAAGGAACTACCACGTGTTCTACTGCATGCTGGAGGGTATGAG
TGAGGATCAGAAGAAGAAGCTGGGCTTGGGCCAGGCCTCTGACTACAACTACTTGGCCATGGGTAACTGCATAACCT
GTGAGGGCCGGGTGGACAGCCAGGAGTACGCCAACATCCGCTCCGCCATGAAGGTGCTCATGTTCACTGACACCGAG
AACTGGGAGATCTCGAAGCTCCTGGCTGCCATCCTGCACCTGGGCAACCTGCAGTATGAGGCACGCACATTTGAAAA
CCTGGATGCCTGTGAGGTTCTCTTCTCCCCATCGCTGGCCACAGCTGCATCCCTGCTTGAGGTGAACCCCCCAGACC
TGATGAGCTGCCTGACTAGCCGCACCCTCATCACCCGCGGGGAGACGGTGTCCACCCCACTGAGCAGGGAACAGGCA
CTGGACGTGCGCGACGCCTTCGTAAAGGGGATCTACGGGCGGCTGTTCGTGTGGATTGTGGACAAGATCAACGCAGC
AATTTACAAGCCTCCCTCCCAGGATGTGAAGAACTCTCGCAGGTCCATCGGCCTCCTGGACATCTTTGGGTTTGAGA
ACTTTGCTGTGAACAGCTTTGAGCAGCTCTGCATCAACTTCGCCAATGAGCACCTGCAGCAGTTCTTTGTGCGGCAC
GTGTTCAAGCTGGAGCAGGAGGAATATGACCTGGAGAGCATTGACTGGCTGCACATCGAGTTCACTGACAACCAGGA
TGCCCTGGACATGATTGCCAACAAGCCCATGAACATCATCTCCCTCATCGATGAGGAGAGCAAGTTCCCCAAGGGCA
CAGACACCACCATGTTACACAAGCTGAACTCCCAGCACAAGCTCAACGCCAACTACATCCCCCCCAAGAACAACCAT
GAGACCCAGTTTGGCATCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAGATCTTCCAGGCCGATG
TCGCCATGGGCGCCGAGACCAGGAAGCGCTCGCCCACACTTAGCAGCCAGTTCAAGCGGTCACTGGAGCTGCTGATG
CGCACGCTGGGTGCCTGCCAGCCCTTCTTTGTGCGATGCATCAAGCCCAATGAGTTCAAGAAGCCCATGCTGTTCGA
CCGGCACCTGTGCGTGCGCCAGCTGCGGTACTCAGGAATGATGGAGACCATCCGAATCCGCCGAGCTGGCTACCCCA
TCCGCTACAGCTTCGTAGAGTTTGTGGAGCGGTACCGTGTGCTGCTGCCAGGTGTGAAGCCGGCCTACAAGCAGGGC
GACCTCCGCGGGACTTGCCAGCGCATGGCTGAGGCTGTGCTGGGCACCCACGATGACTGGCAGATAGGCAAAACCAA
GATCTTTCTGAAGGACCACCATGACATGCTGCTGGAAGTGGAGCGGGACAAAGCCATCACCGACAGAGTCATCCTCC
TTCAGAAAGTCATCCGGGGATTCAAAGACAGGTCTAACTTTCTGAAGCTGAAGAACGCTGCCACACTGATCCAGAGG
CACTGGCGGGGTCACAACTGTAGGAAGAACTACGGGCTGATGCGTCTGGGCTTCCTGCGGCTGCAGGCCCTGCACCG
CTCCCGGAAGCTGCACCAGCAGTACCGCCTGGCCCGCCAGCGCATCATCCAGTTCCAGGCCCGCTGCCGCGCCTATC
TGGTGCGCAAGGCCTTCCGCCACCGCCTCTGGGCTGTGCTCACCGTGCAGGCCTATGCCCGGGGCATGATCGCCCGC
AGGCTGCACCAACGCCTCAGGGCTGAGTATCTGTGGCGCCTCGAGGCTGAGAAAATGCGGCTGGCGGAGGAAGAGAA
GCTTCGGAAGGAGATGAGCGCCAAGAAGGCCAAGGAGGAGGCCGAGCGCAAGCATCAGGAGCGCCTGGCCCAGCTGG
CTCGTGAGGACGCTGAGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGGGACTTCAGGTGGCCT
GCCAGGCCAGGAGGGCCAGGCACCTAGTGGCTTTGAGGACCTGGAGCGAGGGCGGAGGGAGATGGTGGAGGAGGACC
TGGATGCAGCCCTGCCCCTGCCTGACGAGGATGAGGAGGACCTCTCTGAGTATAAATTTGCCAAGTTCGCGGCCACC
TACTTCCAGGGGACAACCACGCACTCCTACACCCGGCGGCCACTCAAACAGCCACTGCTCTACCATGACGACGAGGG
TGACCAGCTGGCAGCCCTGGCGGTCTGGATCACCATCCTCCGCTTCATGGGGGACCTCCCTGAGCCCAAGTACCACA
CAGCCATGAGTGATGGCAGTGAGAAGATCCCTGTGATGACCAAGATTTATGAGACCCTGGGCAAGAAGACGTACAAG
AGGGAGCTGCAGGCCCTGCAGGGCGAGGGCGAGGCCCAGCTCCCCGAGGGCCAGAAGAAGAGCAGTGTGAGGCACAA
GCTGGTGCATTTGACTCTGAAAAAGAAGTCCAAGCTCACAGAGGAGGTGACCAAGAGGCTGCATGACGGGGAGTCCA
CAGTGCAGGGCAACAGCATGCTGGAGGACCGGCCCACCTCCAACCTGGAGAAGCTGCACTTCATCATCGGCAATGGC
ATCCTGCGGCCAGCACTCCGGGACGAGATCTACTGCCAGATCAGCAAGCAGCTGACCCACAACCCCTCCAAGAGCAG
CTATGCCCGGGGCTGGATTCTCGTGTCTCTCTGCGTGGGCTGTTTCGCCCCCTCCGAGAAGTTTGTCAAGTACCTGC
GGAACTTCATCCACGGGGGCCCGCCCGGCTACGCCCCGTACTGTGAGGAGCGCCTGAGAAGGACCTTTGTCAATGGG
ACACGGACACAGCCGCCCAGCTGGCTGGAGCTGCAGGCCACCAAGTCCAAGAAGCCAATCATGTTGCCCGTGACATT
CATGGATGGGACCACCAAGACCCTGCTGACGGACTCGGCAACCACGGCCAAGGAGCTCTGCAACGCGCTGGCCGACA
AGATCTCTCTCAAGGACCGGTTCGGGTTCTCCCTCTACATTGCCCTGTTTGACAAGGTGTCCTCCCTGGGCAGCGGC
AGTGACCACGTCATGGACGCCATCTCCCAGTGCGAGCAGTACGCCAAGGAGCAGGGCGCCCAGGAGCGCAACGCCCC
CTGGAGGCTCTTCTTCCGCAAAGAGGTCTTCACGCCCTGGCACAGCCCCTCCGAGGACAACGTGGCCACCAACCTCA
TCTACCAGCAGGTGGTGCGAGGAGTCAAGTTTGGGGAGTACAGGTGTGAGAAGGAGGACGACCTGGCTGAGCTGGCC
TCCCAGCAGTACTTTGTAGACTATGGCTCTGAGATGATCCTGGAGCGCCTCCTGAACCTCGTGCCCACCTACATCCC
CGACCGCGAGATCACGCCCCTGAAGACGCTGGAGAAGTGGGCCCAGCTGGCCATCGCCGCCCACAAGAAGGGGATTT
ATGCCCAGAGGAGAACTGATGCCCAGAAGGTCAAAGAGGATGTGGTCAGTTATGCCCGCTTCAAGTGGCCCTTGCTC
TTCTCCAGGTTTTATGAAGCCTACAAATTCTCAGGCCCCAGTCTCCCCAAGAACGACGTCATCGTGGCCGTCAACTG
GACGGGTGTGTACTTTGTGGATGAGCAGGAGCAGGTACTTCTGGAGCTGTCCTTCCCAGAGATCATGGCCGTGTCCA
GCAGCAGGGGAGCGAAAACGACGGCCCCCAGCTTCACGCTGGCCACCATCAAGGGGGACGAATACACCTTCACCTCC
AGCAATGCTGAGGACATTCGTGACCTGGTGGTCACCTTCCTAGAGGGGCTCCGGAAGAGATCTAAGTATGTTGTGGC
CCTGCAGGATAACCCCAACCCCGCAGGCGAGGAGTCAGGCTTCCTCAGCTTTGCCAAGGGAGACCTCATCATCCTGG
ACCATGACACGGGCGAGCAGGTCATGAACTCGGGCTGGGCCAACGGCATCAATGAGAGGACCAAGCAGCGTGGGGAC
TTCCCCACCGACAGTGTGTACGTCATGCCCACTGTCACCATGCCACCGCGGGAGATTGTGGCCCTGGTCACCATGAC
TCCCGATCAGAGGCAGGACGTTGTCCGGCTCTTGCAGCTGCGAACGGCGGAGCCCGAGGTGCGTGCCAAGCCCTACA
CGCTGGAGGAGTTTTCCTATGACTACTTCAGGCCCCCACCCAAGCACACGCTGAGCCGTGTCATGGTGTCCAAGGCC
CGAGGCAAGGACCGGCTGTGGAGCCACACGCGGGAACCGCTCAAGCAGGCGCTGCTCAAGAAGCTCCTGGGCAGTGA
GGAGCTCTCGCAGGAGGCCTGCCTGGCCTTCATTGCTGTGCTCAAGTACATGGGCGACTACCCGTCCAAGAGGACAC
GCTCCGTCAACGAGCTCACCGACCAGATCTTTGAGGGTCCCCTGAAAGCCGAGCCCCTGAAGGACGAGGCATATGTG
CAGATCCTGAAGCAGCTGACCGACAACCACATCAGGTACAGCGAGGAGCGGGGTTGGGAGCTGCTCTGGCTGTGCAC
GGGCCTTTTCCCACCCAGCAACATCCTCCTGCCCCACGTGCAGCGCTTCCTGCAGTCCCGAAAGCACTGCCCACTCG
CCATCGACTGCCTGCAACGGCTCCAGAAAGCCCTGAGAAACGGGTCCCGGAAGTACCCTCCGCACCTGGTGGAGGTG
GAGGCCATCCAGCACAAGACCACCCAGATTTTCCACAAAGTCTACTTCCCTGATGACACTGACGAGGCCTTCGAAGT
GGAGTCCAGCACCAAGGCCAAGGACTTCTGCCAGAACATCGCCACCAGGCTGCTCCTCAAGTCCTCAGAGGGATTCA
GCCTCTTTGTCAAAATTGCAGACAAGGTCCTCAGCGTTCCTGAGAATGACTTCTTCTTTGACTTTGTTCGACACTTG
ACAGACTGGATAAAGAAAGCTCGGCCCATCAAGGACGGAATTGTGCCCTCACTCACCTACCAGGTGTTCTTCATGAA
GAAGCTGTGGACCACCACGGTGCCAGGGAAGGATCCCATGGCCGATTCCATCTTCCACTATTACCAGGAGTTGCCCA
AGTATCTCCGAGGCTACCACAAGTGCACGCGGGAGGAGGTGCTGCAGCTGGGGGCGCTGATCTACAGGGTCAAGTTC
GAGGAGGACAAGTCCTACTTCCCCAGCATCCCCAAGCTGCTGCGGGAGCTGGTGCCCCAGGACCTTATCCGGCAGGT
CTCACCTGATGACTGGAAGCGGTCCATCGTCGCCTACTTCAACAAGCACGCAGGGAAGTCCAAGGAGGAGGCCAAGC
TGGCCTTCCTGAAGCTCATCTTCAAGTGGCCCACCTTTGGCTCAGCCTTCTTCGAGCAAACTACGGAGCCAAACTTC
CCTGAGATCCTCCTAATTGCCATCAACAAGTATGGGGTCAGCCTCATCGATCCCAAAACGAAGGATATCCTCACCAC
TCATCCCTTCACCAAGATCTCCAACTGGAGCAGCGGCAACACCTACTTCCACATCACCATTGGGAACTTGGTGCGCG
GGAGCAAACTGCTCTGCGAGACGTCACTGGGCTACAAGATGGATGACCTCCTGACTTCCTACATTAGCCAGATGCTC
ACAGCCATGAGCAAACAGCGGGGCTCCAGGAGCGGCAAGTGAACAGTCACGGGGAGGTGCTGGTTCCATGCCTGCTC
TCGAGGCAGCAGTGGGTTCAGGCCCATCAGCTACCCCTGCAGCTGGGGAAGACTTATGCCATCCCGGCAGCGAGGCT
GGGCTGGCCAGCCACCACTGACTATACCAACTGGGCCTCTGATGTTCTTCCAGTGAGGCATCTCTCTGGGATGCAGA
ACTTCCCTCCATCCACCCCTCTGGCACCTGGGTTGGTCTAATCCTAGTTTGCTGTGGCCTTCCCGGTTGTGAGAGCC
TGTGATCCTTAGATGTGTCTCCTGTTTCAGACCAGCCCCACCATGCAACTTCCTTTGACTTTCTGTGTACCACTGGG
ATAGAGGAATCAAGAGGACAATCTAGCTCTCCATACTTTGAACAACCAAATGTGCATTGAATACTCTGAAACCGAAG
GGACTGGATCTGCAGGTGGGATGAGGGAGACAGACCACTTTTCTATATTGCAGTGTGAATGCTGGGCCCCTGCTCAA
GTCTACCCTGATCACCTCAGGGCATAAAGCATGTTTCATTCTCTGGCC (SEQ ID NO: 3)
Homo sapiens myosin VIIA (MYO7A), isoform 2, protein
NCBI RefSeq NP_001120652.1
MVILQQGDHVWMDLRLGQEFDVPIGAVVKLCDSGQVQVVDDEDNEHWISPQNATHIKPMHPTSVHGVEDMIRLGDLN
EAGILRNLLIRYRDHLIYTYTGSILVAVNPYQLLSIYSPEHIRQYTNKKIGEMPPHIFAIADNCYFNMKRNSRDQCC
IISGESGAGKTESTKLILQFLAAISGQHSWIEQQVLEATPILEAFGNAKTIRNDNSSRFGKYIDIHENKRGAIEGAK
IEQYLLEKSRVCRQALDERNYHVFYCMLEGMSEDQKKKLGLGQASDYNYLAMGNCITCEGRVDSQEYANIRSAMKVL
MFTDTENWEISKLLAAILHLGNLQYEARTFENLDACEVLFSPSLATAASLLEVNPPDLMSCLTSRTLITRGETVSTP
LSREQALDVRDAFVKGIYGRLFVWIVDKINAAIYKPPSQDVKNSRRSIGLLDIFGFENFAVNSFEQLCINFANEHLQ
QFFVRHVFKLEQEEYDLESIDWLHIEFTDNQDALDMIANKPMNIISLIDEESKFPKGTDTTMLHKLNSQHKLNANYI
PPKNNHETQFGINHFAGIVYYETQGFLEKNRDTLHGDIIQLVHSSRNKFIKQIFQADVAMGAETRKRSPTLSSQFKR
SLELLMRTLGACQPFFVRCIKPNEFKKPMLFDRHLCVRQLRYSGMMETIRIRRAGYPIRYSFVEFVERYRVLLPGVK
PAYKQGDLRGTCQRMAEAVLGTHDDWQIGKTKIFLKDHHDMLLEVERDKAITDRVILLQKVIRGFKDRSNFLKLKNA
ATLIQRHWRGHNCRKNYGLMRLGFLRLQALHRSRKLHQQYRLARQRIIQFQARCRAYLVRKAFRHRLWAVLTVQAYA
RGMIARRLHQRLRAEYLWRLEAEKMRLAEEEKLRKEMSAKKAKEEAERKHQERLAQLAREDAERELKEKEAARRKKE
LLEQMERARHEPVNHSDMVDKMFGFLGTSGGLPGQEGQAPSGFEDLERGRREMVEEDLDAALPLPDEDEEDLSEYKF
AKFAATYFQGTTTHSYTRRPLKQPLLYHDDEGDQLAALAVWITILRFMGDLPEPKYHTAMSDGSEKIPVMTKIYETL
GKKTYKRELQALQGEGEAQLPEGQKKSSVRHKLVHLTLKKKSKLTEEVTKRLHDGESTVQGNSMLEDRPTSNLEKLH
FIIGNGILRPALRDEIYCQISKQLTHNPSKSSYARGWILVSLCVGCFAPSEKFVKYLRNFIHGGPPGYAPYCEERLR
RTFVNGTRTQPPSWLELQATKSKKPIMLPVTFMDGTTKILLTDSATTAKELCNALADKISLKDRFGFSLYIALFDKV
SSLGSGSDHVMDAISQCEQYAKEQGAQERNAPWRLFFRKEVFTPWHSPSEDNVATNLIYQQVVRGVKFGEYRCEKED
DLAELASQQYFVDYGSEMILERLLNLVPTYIPDREITPLKTLEKWAQLAIAAHKKGIYAQRRIDAQKVKEDVVSYAR
FKWPLLFSRFYEAYKFSGPSLPKNDVIVAVNWTGVYFVDEQEQVLLELSFPEIMAVSSSRGAKITAPSFTLATIKGD
EYTFTSSNAEDIRDLVVTFLEGLRKRSKYVVALQDNPNPAGEESGFLSFAKGDLIILDHDTGEQVMNSGWANGINER
TKQRGDFPTDSVYVMPTVTMPPREIVALVTMTPDQRQDVVRLLQLRTAEPEVRAKPYTLEEFSYDYFRPPPKHTLSR
VMVSKARGKDRLWSHTREPLKQALLKKLLGSEELSQEACLAFIAVLKYMGDYPSKRTRSVNELTDQIFEGPLKAEPL
KDEAYVQILKQLTDNHIRYSEERGWELLWLCTGLFPPSNILLPHVQRFLQSRKHCPLAIDCLQRLQKALRNGSRKYP
PHLVEVEAIQHKTTQIFHKVYFPDDTDEAFEVESSTKAKDFCQNIATRLLLKSSEGFSLFVKIADKVLSVPENDFFF
DFVRHLTDWIKKARPIKDGIVPSLTYQVFFMKKLWTTTVPGKDPMADSIFHYYQELPKYLRGYHKCTREEVLQLGAL
IYRVKFEEDKSYFPSIPKLLRELVPQDLIRQVSPDDWKRSIVAYFNKHAGKSKEEAKLAFLKLIFKWPTFGSAFFEQ
TTEPNFPEILLIAINKYGVSLIDPKTKDILTTHPFTKISNWSSGNTYFHITIGNLVRGSKLLCETSLGYKMDDLLTS
YISQMLTAMSKQRGSRSGK (SEQ ID NO: 4)
Homo sapiens myosin VIIA (MYO7A), transcript variant 4, mRNA
NCBI RefSeq NM_001369365.1
AGTGCTGGCTGGACAGCTGCTCTGGGCAGGAGAGAGAGGGAGAGACAAGAGACACACACAGAGAGACGGCGAGGAAG
GGAAAGACCCAGAGGGACGCCTAGAACGAGACTTGGAGCCAGACAGAGGAAGAGGGGACGTGTGTTTGCAGACTGGC
TGGGCCCGTGACCCAGCTTCCTGAGTCCTCCGTGCAGGTGGCAGCTGTACCAGGCTGGCAGGTCACTGAGAGTGGGC
AGCTGGGCCCCAGAACTGTGCCTGGCCCAGTGGGCAGCAGGAGCTCCTGACTTGGGACCATGGTGATTCTTCAGCAG
GAGAAAAGCTGGGACTTGCCCCACGTTACACAGCCACACTGAGGCATGTGTCGTGTCACAAACGAGATCTTCCATTC
AAGGGGGACCATGTGTGGATGGACCTGAGATTGGGGCAGGAGTTCGACGTGCCCATCGGGGCGGTGGTGAAGCTCTG
CGACTCTGGGCAGGTCCAGGTGGTGGATGATGAAGACAATGAACACTGGATCTCTCCGCAGAACGCAACGCACATCA
AGCCTATGCACCCCACGTCGGTCCACGGCGTGGAGGACATGATCCGCCTGGGGGACCTCAACGAGGCGGGCATCTTG
CGCAACCTGCTTATCCGCTACCGGGACCACCTCATCTACACGTATACGGGCTCCATCCTGGTGGCTGTGAACCCCTA
CCAGCTGCTCTCCATCTACTCGCCAGAGCACATCCGCCAGTATACCAACAAGAAGATTGGGGAGATGCCCCCCCACA
TCTTTGCCATTGCTGACAACTGCTACTTCAACATGAAACGCAACAGCCGAGACCAGTGCTGCATCATCAGTGGGGAA
TCTGGGGCCGGGAAGACGGAGAGCACAAAGCTGATCCTGCAGTTCCTGGCAGCCATCAGTGGGCAGCACTCGTGGAT
TGAGCAGCAGGTCTTGGAGGCCACCCCCATTCTGGAAGCATTTGGGAATGCCAAGACCATCCGCAATGACAACTCAA
GCCGTTTCGGAAAGTACATCGACATCCACTTCAACAAGCGGGGCGCCATCGAGGGCGCGAAGATTGAGCAGTACCTG
CTGGAAAAGTCACGTGTCTGTCGCCAGGCCCTGGATGAAAGGAACTACCACGTGTTCTACTGCATGCTGGAGGGTAT
GAGTGAGGATCAGAAGAAGAAGCTGGGCTTGGGCCAGGCCTCTGACTACAACTACTTGGCCATGGGTAACTGCATAA
CCTGTGAGGGCCGGGTGGACAGCCAGGAGTACGCCAACATCCGCTCCGCCATGAAGGTGCTCATGTTCACTGACACC
GAGAACTGGGAGATCTCGAAGCTCCTGGCTGCCATCCTGCACCTGGGCAACCTGCAGTATGAGGCACGCACATTTGA
AAACCTGGATGCCTGTGAGGTTCTCTTCTCCCCATCGCTGGCCACAGCTGCATCCCTGCTTGAGGTGAACCCCCCAG
ACCTGATGAGCTGCCTGACTAGCCGCACCCTCATCACCCGCGGGGAGACGGTGTCCACCCCACTGAGCAGGGAACAG
GCACTGGACGTGCGCGACGCCTTCGTAAAGGGGATCTACGGGCGGCTGTTCGTGTGGATTGTGGACAAGATCAACGC
AGCAATTTACAAGCCTCCCTCCCAGGATGTGAAGAACTCTCGCAGGTCCATCGGCCTCCTGGACATCTTTGGGTTTG
AGAACTTTGCTGTGAACAGCTTTGAGCAGCTCTGCATCAACTTCGCCAATGAGCACCTGCAGCAGTTCTTTGTGCGG
CACGTGTTCAAGCTGGAGCAGGAGGAATATGACCTGGAGAGCATTGACTGGCTGCACATCGAGTTCACTGACAACCA
GGATGCCCTGGACATGATTGCCAACAAGCCCATGAACATCATCTCCCTCATCGATGAGGAGAGCAAGTTCCCCAAGG
GCACAGACACCACCATGTTACACAAGCTGAACTCCCAGCACAAGCTCAACGCCAACTACATCCCCCCCAAGAACAAC
CATGAGACCCAGTTTGGCATCAACCATTTTGCAGGCATCGTCTACTATGAGACCCAAGGCTTCCTGGAGAAGAACCG
AGACACCCTGCATGGGGACATTATCCAGCTGGTCCACTCCTCCAGGAACAAGTTCATCAAGCAGATCTTCCAGGCCG
ATGTCGCCATGGGCGCCGAGACCAGGAAGCGCTCGCCCACACTTAGCAGCCAGTTCAAGCGGTCACTGGAGCTGCTG
ATGCGCACGCTGGGTGCCTGCCAGCCCTTCTTTGTGCGATGCATCAAGCCCAATGAGTTCAAGAAGCCCATGCTGTT
CGACCGGCACCTGTGCGTGCGCCAGCTGCGGTACTCAGGAATGATGGAGACCATCCGAATCCGCCGAGCTGGCTACC
CCATCCGCTACAGCTTCGTAGAGTTTGTGGAGCGGTACCGTGTGCTGCTGCCAGGTGTGAAGCCGGCCTACAAGCAG
GGCGACCTCCGCGGGACTTGCCAGCGCATGGCTGAGGCTGTGCTGGGCACCCACGATGACTGGCAGATAGGCAAAAC
CAAGATCTTTCTGAAGGACCACCATGACATGCTGCTGGAAGTGGAGCGGGACAAAGCCATCACCGACAGAGTCATCC
TCCTTCAGAAAGTCATCCGGGGATTCAAAGACAGGTCTAACTTTCTGAAGCTGAAGAACGCTGCCACACTGATCCAG
AGGCACTGGCGGGGTCACAACTGTAGGAAGAACTACGGGCTGATGCGTCTGGGCTTCCTGCGGCTGCAGGCCCTGCA
CCGCTCCCGGAAGCTGCACCAGCAGTACCGCCTGGCCCGCCAGCGCATCATCCAGTTCCAGGCCCGCTGCCGCGCCT
ATCTGGTGCGCAAGGCCTTCCGCCACCGCCTCTGGGCTGTGCTCACCGTGCAGGCCTATGCCCGGGGCATGATCGCC
CGCAGGCTGCACCAACGCCTCAGGGCTGAGTATCTGTGGCGCCTCGAGGCTGAGAAAATGCGGCTGGCGGAGGAAGA
GAAGCTTCGGAAGGAGATGAGCGCCAAGAAGGCCAAGGAGGAGGCCGAGCGCAAGCATCAGGAGCGCCTGGCCCAGC
TGGCTCGTGAGGACGCTGAGCGGGAGCTGAAGGAGAAGGAGGCCGCTCGGCGGAAGAAGGAGCTCCTGGAGCAGATG
GAAAGGGCCCGCCATGAGCCTGTCAATCACTCAGACATGGTGGACAAGATGTTTGGCTTCCTGGGGACTTCAGGTGG
CCTGCCAGGCCAGGAGGGCCAGGCACCTAGTGGCTTTGAGGACCTGGAGCGAGGGCGGAGGGAGATGGTGGAGGAGG
ACCTGGATGCAGCCCTGCCCCTGCCTGACGAGGATGAGGAGGACCTCTCTGAGTATAAATTTGCCAAGTTCGCGGCC
ACCTACTTCCAGGGGACAACCACGCACTCCTACACCCGGCGGCCACTCAAACAGCCACTGCTCTACCATGACGACGA
GGGTGACCAGCTGGCAGCCCTGGCGGTCTGGATCACCATCCTCCGCTTCATGGGGGACCTCCCTGAGCCCAAGTACC
ACACAGCCATGAGTGATGGCAGTGAGAAGATCCCTGTGATGACCAAGATTTATGAGACCCTGGGCAAGAAGACGTAC
AAGAGGGAGCTGCAGGCCCTGCAGGGCGAGGGCGAGGCCCAGCTCCCCGAGGGCCAGAAGAAGAGCAGTGTGAGGCA
CAAGCTGGTGCATTTGACTCTGAAAAAGAAGTCCAAGCTCACAGAGGAGGTGACCAAGAGGCTGCATGACGGGGAGT
CCACAGTGCAGGGCAACAGCATGCTGGAGGACCGGCCCACCTCCAACCTGGAGAAGCTGCACTTCATCATCGGCAAT
GGCATCCTGCGGCCAGCACTCCGGGACGAGATCTACTGCCAGATCAGCAAGCAGCTGACCCACAACCCCTCCAAGAG
CAGCTATGCCCGGGGCTGGATTCTCGTGTCTCTCTGCGTGGGCTGTTTCGCCCCCTCCGAGAAGTTTGTCAAGTACC
TGCGGAACTTCATCCACGGGGGCCCGCCCGGCTACGCCCCGTACTGTGAGGAGCGCCTGAGAAGGACCTTTGTCAAT
GGGACACGGACACAGCCGCCCAGCTGGCTGGAGCTGCAGGCCACCAAGTCCAAGAAGCCAATCATGTTGCCCGTGAC
ATTCATGGATGGGACCACCAAGACCCTGCTGACGGACTCGGCAACCACGGCCAAGGAGCTCTGCAACGCGCTGGCCG
ACAAGATCTCTCTCAAGGACCGGTTCGGGTTCTCCCTCTACATTGCCCTGTTTGACAAGGTGTCCTCCCTGGGCAGC
GGCAGTGACCACGTCATGGACGCCATCTCCCAGTGCGAGCAGTACGCCAAGGAGCAGGGCGCCCAGGAGCGCAACGC
CCCCTGGAGGCTCTTCTTCCGCAAAGAGGTCTTCACGCCCTGGCACAGCCCCTCCGAGGACAACGTGGCCACCAACC
TCATCTACCAGCAGGTGGTGCGAGGAGTCAAGTTTGGGGAGTACAGGTGTGAGAAGGAGGACGACCTGGCTGAGCTG
GCCTCCCAGCAGTACTTTGTAGACTATGGCTCTGAGATGATCCTGGAGCGCCTCCTGAACCTCGTGCCCACCTACAT
CCCCGACCGCGAGATCACGCCCCTGAAGACGCTGGAGAAGTGGGCCCAGCTGGCCATCGCCGCCCACAAGAAGGGGA
TTTATGCCCAGAGGAGAACTGATGCCCAGAAGGTCAAAGAGGATGTGGTCAGTTATGCCCGCTTCAAGTGGCCCTTG
CTCTTCTCCAGGTTTTATGAAGCCTACAAATTCTCAGGCCCCAGTCTCCCCAAGAACGACGTCATCGTGGCCGTCAA
CTGGACGGGTGTGTACTTTGTGGATGAGCAGGAGCAGGTACTTCTGGAGCTGTCCTTCCCAGAGATCATGGCCGTGT
CCAGCAGCAGGGGAGCGAAAACGACGGCCCCCAGCTTCACGCTGGCCACCATCAAGGGGGACGAATACACCTTCACC
TCCAGCAATGCTGAGGACATTCGTGACCTGGTGGTCACCTTCCTAGAGGGGCTCCGGAAGAGATCTAAGTATGTTGT
GGCCCTGCAGGATAACCCCAACCCCGCAGGCGAGGAGTCAGGCTTCCTCAGCTTTGCCAAGGGAGACCTCATCATCC
TGGACCATGACACGGGCGAGCAGGTCATGAACTCGGGCTGGGCCAACGGCATCAATGAGAGGACCAAGCAGCGTGGG
GACTTCCCCACCGACAGTGTGTACGTCATGCCCACTGTCACCATGCCACCGCGGGAGATTGTGGCCCTGGTCACCAT
GACTCCCGATCAGAGGCAGGACGTTGTCCGGCTCTTGCAGCTGCGAACGGCGGAGCCCGAGGTGCGTGCCAAGCCCT
ACACGCTGGAGGAGTTTTCCTATGACTACTTCAGGCCCCCACCCAAGCACACGCTGAGCCGTGTCATGGTGTCCAAG
GCCCGAGGCAAGGACCGGCTGTGGAGCCACACGCGGGAACCGCTCAAGCAGGCGCTGCTCAAGAAGCTCCTGGGCAG
TGAGGAGCTCTCGCAGGAGGCCTGCCTGGCCTTCATTGCTGTGCTCAAGTACATGGGCGACTACCCGTCCAAGAGGA
CACGCTCCGTCAACGAGCTCACCGACCAGATCTTTGAGGGTCCCCTGAAAGCCGAGCCCCTGAAGGACGAGGCATAT
GTGCAGATCCTGAAGCAGCTGACCGACAACCACATCAGGTACAGCGAGGAGCGGGGTTGGGAGCTGCTCTGGCTGTG
CACGGGCCTTTTCCCACCCAGCAACATCCTCCTGCCCCACGTGCAGCGCTTCCTGCAGTCCCGAAAGCACTGCCCAC
TCGCCATCGACTGCCTGCAACGGCTCCAGAAAGCCCTGAGAAACGGGTCCCGGAAGTACCCTCCGCACCTGGTGGAG
GTGGAGGCCATCCAGCACAAGACCACCCAGATTTTCCACAAAGTCTACTTCCCTGATGACACTGACGAGGCCTTCGA
AGTGGAGTCCAGCACCAAGGCCAAGGACTTCTGCCAGAACATCGCCACCAGGCTGCTCCTCAAGTCCTCAGAGGGAT
TCAGCCTCTTTGTCAAAATTGCAGACAAGGTCCTCAGCGTTCCTGAGAATGACTTCTTCTTTGACTTTGTTCGACAC
TTGACAGACTGGATAAAGAAAGCTCGGCCCATCAAGGACGGAATTGTGCCCTCACTCACCTACCAGGTGTTCTTCAT
GAAGAAGCTGTGGACCACCACGGTGCCAGGGAAGGATCCCATGGCCGATTCCATCTTCCACTATTACCAGGAGTTGC
CCAAGTATCTCCGAGGCTACCACAAGTGCACGCGGGAGGAGGTGCTGCAGCTGGGGGCGCTGATCTACAGGGTCAAG
TTCGAGGAGGACAAGTCCTACTTCCCCAGCATCCCCAAGCTGCTGCGGGAGCTGGTGCCCCAGGACCTTATCCGGCA
GGTCTCACCTGATGACTGGAAGCGGTCCATCGTCGCCTACTTCAACAAGCACGCAGGGAAGTCCAAGGAGGAGGCCA
AGCTGGCCTTCCTGAAGCTCATCTTCAAGTGGCCCACCTTTGGCTCAGCCTTCTTCGAGGTGAAGCAAACTACGGAG
CCAAACTTCCCTGAGATCCTCCTAATTGCCATCAACAAGTATGGGGTCAGCCTCATCGATCCCAAAACGAAGGATAT
CCTCACCACTCATCCCTTCACCAAGATCTCCAACTGGAGCAGCGGCAACACCTACTTCCACATCACCATTGGGAACT
TGGTGCGCGGGAGCAAACTGCTCTGCGAGACGTCACTGGGCTACAAGATGGATGACCTCCTGACTTCCTACATTAGC
CAGATGCTCACAGCCATGAGCAAACAGCGGGGCTCCAGGAGCGGCAAGTGAACAGTCACGGGGAGGTGCTGGTTCCA
TGCCTGCTCTCGAGGCAGCAGTGGGTTCAGGCCCATCAGCTACCCCTGCAGCTGGGGAAGACTTATGCCATCCCGGC
AGCGAGGCTGGGCTGGCCAGCCACCACTGACTATACCAACTGGGCCTCTGATGTTCTTCCAGTGAGGCATCTCTCTG
GGATGCAGAACTTCCCTCCATCCACCCCTCTGGCACCTGGGTTGGTCTAATCCTAGTTTGCTGTGGCCTTCCCGGTT
GTGAGAGCCTGTGATCCTTAGATGTGTCTCCTGTTTCAGACCAGCCCCACCATGCAACTTCCTTTGACTTTCTGTGT
ACCACTGGGATAGAGGAATCAAGAGGACAATCTAGCTCTCCATACTTTGAACAACCAAATGTGCATTGAATACTCTG
AAACCGAAGGGACTGGATCTGCAGGTGGGATGAGGGAGACAGACCACTTTTCTATATTGCAGTGTGAATGCTGGGCC
CCTGCTCAAGTCTACCCTGATCACCTCAGGGCATAAAGCATGTTTCATTCTCTGGCC (SEQ ID NO: 5)
Homo sapiens myosin VIIA (MYO7A), isoform 4, protein
NCBI RefSeq NP_001356294.1
MDLRLGQEFDVPIGAVVKLCDSGQVQVVDDEDNEHWISPQNATHIKPMHPTSVHGVEDMIRLGDLNEAGILRNLLIR
YRDHLIYTYTGSILVAVNPYQLLSIYSPEHIRQYTNKKIGEMPPHIFAIADNCYFNMKRNSRDQCCIISGESGAGKT
ESTKLILQFLAAISGQHSWIEQQVLEATPILEAFGNAKTIRNDNSSRFGKYIDIHFNKRGAIEGAKIEQYLLEKSRV
CRQALDERNYHVFYCMLEGMSEDQKKKLGLGQASDYNYLAMGNCITCEGRVDSQEYANIRSAMKVLMFTDTENWEIS
KLLAAILHLGNLQYEARTFENLDACEVLFSPSLATAASLLEVNPPDLMSCLTSRTLITRGETVSTPLSREQALDVRD
AFVKGIYGRLFVWIVDKINAAIYKPPSQDVKNSRRSIGLLDIFGFENFAVNSFEQLCINFANEHLQQFFVRHVFKLE
QEEYDLESIDWLHIEFTDNQDALDMIANKPMNIISLIDEESKFPKGTDTTMLHKLNSQHKLNANYIPPKNNHETQFG
INHFAGIVYYETQGFLEKNRDTLHGDIIQLVHSSRNKFIKQIFQADVAMGAETRKRSPTLSSQFKRSLELLMRTLGA
CQPFFVRCIKPNEFKKPMLFDRHLCVRQLRYSGMMETIRIRRAGYPIRYSFVEFVERYRVLLPGVKPAYKQGDLRGT
CQRMAEAVLGTHDDWQIGKTKIFLKDHHDMLLEVERDKAITDRVILLQKVIRGFKDRSNFLKLKNAATLIQRHWRGH
NCRKNYGLMRLGFLRLQALHRSRKLHQQYRLARQRIIQFQARCRAYLVRKAFRHRLWAVLTVQAYARGMIARRLHQR
LRAEYLWRLEAEKMRLAEEEKLRKEMSAKKAKEEAERKHQERLAQLAREDAERELKEKEAARRKKELLEQMERARHE
PVNHSDMVDKMFGFLGTSGGLPGQEGQAPSGFEDLERGRREMVEEDLDAALPLPDEDEEDLSEYKFAKFAATYFQGT
TTHSYTRRPLKQPLLYHDDEGDQLAALAVWITILRFMGDLPEPKYHTAMSDGSEKIPVMTKIYETLGKKTYKRELQA
LQGEGEAQLPEGQKKSSVRHKLVHLTLKKKSKLTEEVTKRLHDGESTVQGNSMLEDRPTSNLEKLHFIIGNGILRPA
LRDEIYCQISKQLTHNPSKSSYARGWILVSLCVGCFAPSEKFVKYLRNFIHGGPPGYAPYCEERLRRTFVNGTRTQP
PSWLELQATKSKKPIMLPVTFMDGTTKTLLTDSATTAKELCNALADKISLKDRFGFSLYIALFDKVSSLGSGSDHVM
DAISQCEQYAKEQGAQERNAPWRLFFRKEVFTPWHSPSEDNVATNLIYQQVVRGVKFGEYRCEKEDDLAELASQQYF
VDYGSEMILERLLNLVPTYIPDREITPLKTLEKWAQLAIAAHKKGIYAQRRIDAQKVKEDVVSYARFKWPLLFSRFY
EAYKFSGPSLPKNDVIVAVNWTGVYFVDEQEQVLLELSFPEIMAVSSSRGAKTTAPSFTLATIKGDEYTFTSSNAED
IRDLVVTFLEGLRKRSKYVVALQDNPNPAGEESGFLSFAKGDLIILDHDTGEQVMNSGWANGINERTKQRGDFPTDS
VYVMPTVTMPPREIVALVTMTPDQRQDVVRLLQLRTAEPEVRAKPYTLEEFSYDYFRPPPKHTLSRVMVSKARGKDR
LWSHTREPLKQALLKKLLGSEELSQEACLAFIAVLKYMGDYPSKRTRSVNELTDQIFEGPLKAEPLKDEAYVQILKQ
LTDNHIRYSEERGWELLWLCTGLFPPSNILLPHVQRFLQSRKHCPLAIDCLQRLQKALRNGSRKYPPHLVEVEAIQH
KTTQIFHKVYFPDDTDEAFEVESSTKAKDFCQNIATRLLLKSSEGFSLFVKIADKVLSVPENDFFFDFVRHLTDWIK
KARPIKDGIVPSLTYQVFFMKKLWTTTVPGKDPMADSIFHYYQELPKYLRGYHKCTREEVLQLGALIYRVKFEEDKS
YFPSIPKLLRELVPQDLIRQVSPDDWKRSIVAYFNKHAGKSKEEAKLAFLKLIFKWPTFGSAFFEVKQTTEPNFPEI
LLIAINKYGVSLIDPKTKDILTTHPFTKISNWSSGNTYFHITIGNLVRGSKLLCETSLGYKMDDLLTSYISQMLTAM
SKQRGSRSGK (SEQ ID NO: 6)
Mus musculus myosin VIIA (Myo7a), transcript variant 1, mRNA
NCBI RefSeq NM_001256081.1
AGTGCAGGCTGGACAGCTGCCCTGAACAGAAAGAAAGAGTGACCCAGGGAGACAAGAAACAGAGTAGCCCAAGGGAA
GCCCACAGCAGCAGCAGATCAAGGCTCAAGCTGGAGCTGAAAATTTGCAGGCTCCAGCCTCAGCTTCCAGAGTCCTC
CTGACCTGTGACCCCTGGCTCCTGGCTGGGAGGTGGTGACTCGGAGGGTGTGGATAAAACCCAGAGCTGTGTCTGGT
CACTCCGGCAGGTGTGCTGACGTAGAAGCATGGTTATTCTGCAGAAGGGGGACTATGTATGGATGGACCTGAAGTCA
GGCCAGGAGTTTGATGTGCCCATCGGGGCCGTGGTGAAGCTCTGCGACTCGGGCCAGATCCAGGTGGTGGATGATGA
AGACAATGAACACTGGATATCCCCTCAGAATGCCACGCACATCAAGCCAATGCACCCCACATCGGTGCACGGCGTGG
AGGACATGATCCGCCTGGGGGATCTCAACGAGGCAGGCATCCTTCGAAACCTTCTCATTCGCTACCGGGACCACCTC
ATCTATACGTACACAGGTTCCATCCTGGTGGCCGTGAACCCCTACCAGCTGCTCTCCATCTACTCGCCAGAGCACAT
CCGCCAGTACACCAACAAGAAGATAGGGGAGATGCCCCCCCACATCTTCGCCATTGCTGACAACTGCTACTTCAACA
TGAAACGCAACAACCGGGACCAGTGCTGTATTATCAGCGGGGAGTCGGGAGCTGGCAAGACAGAGAGCACAAAGTTG
ATCCTGCAGTTCCTGGCAGCCATCAGTGGACAGCACTCATGGATCGAGCAGCAGGTGCTGGAGGCCACCCCGATCCT
GGAAGCATTTGGGAACGCCAAGACCATCCGCAACGACAACTCTAGCCGCTTTGGCAAGTACATTGACATCCACTTTA
ACAAGCGTGGTGCCATCGAGGGCGCCAAAATAGAGCAATACCTGCTGGAGAAGTCACGTGTCTGCCGCCAGGCCCCT
GACGAGAGGAACTATCACGTGTTCTACTGTATGCTGGAGGGCATGAATGAGGAGGAGAAGAAGAAACTGGGCCTAGG
CCAGGCCGCTGACTACAACTACTTGGCCATGGGTAACTGCATCACCTGTGAGGGCCGCGTGGACAGTCAGGAGTATG
CCAACATCCGCTCTGCCATGAAGGTTCTCATGTTCACAGACACGGAGAACTGGGAGATCTCGAAGCTTCTGGCTGCC
ATCCTACACATGGGCAATCTGCAGTATGAGGCCCGGACATTTGAGAACTTGGATGCGTGTGAAGTCCTCTTCTCCCC
ATCGCTGGCCACGGCAGCTTCTCTGCTCGAGGTGAACCCCCCAGACCTGATGAGCTGCCTCACCAGCCGCACCCTCA
TCACCCGTGGGGAGACGGTGTCCACCCCTCTCAGCAGGGAACAGGCGCTGGATGTGCGAGATGCCTTTGTCAAGGGC
ATCTATGGGCGGCTCTTTGTGTGGATTGTGGAGAAGATCAACGCAGCAATCTACAAGCCACCCCCCCTGGAAGTGAA
GAACTCTCGCCGGTCCATCGGTCTCCTGGACATCTTTGGATTTGAGAACTTCACTGTGAACAGCTTCGAGCAGCTCT
GCATTAACTTTGCCAATGAGCACCTGCAGCAATTCTTCGTGCGGCACGTGTTCAAGCTGGAGCAGGAGGAGTACGAC
CTGGAGAGCATCGACTGGTTGCACATTGAGTTCACTGACAACCAGGAAGCACTGGACATGATTGCCAACCGGCCTAT
GAACGTCATCTCCCTCATCGATGAGGAGAGCAAGTTCCCCAAGGGCACGGATGCCACCATGCTGCATAAGCTGAACT
CACAGCACAAGCTCAATGCCAACTACGTGCCACCCAAGAACAGCCACGAGACCCAGTTTGGAATCAACCACTTTGCG
GGTGTTGTCTATTATGAGAGTCAAGGCTTCCTGGAGAAGAACCGAGACACCCTGCATGGGGACATCATCCAGCTGGT
CCACTCTTCCCGGAACAAGTTCATAAAGCAGATTTTCCAAGCTGACGTTGCCATGGGTGCCGAGACCAGGAAGCGCT
CGCCTACACTCAGCAGCCAGTTCAAGCGGTCTCTGGAGCTGCTGATGCGCACACTGGGCGCCTGCCAGCCCTTCTTT
GTGCGTTGTATCAAACCCAATGAGTTCAAGAAGCCCATGCTCTTCGACCGGCACTTGTGTGTACGCCAGCTGCGATA
TTCGGGCATGATGGAGACAATCCGCATCCGCCACGCAGGCTACCCCATTCGCTACAGCTTTGTGGAGTTTGTGGAGC
GCTACCGGGTACTGCTGCCTGGTGTGAAGCCAGCATACAAGCAGGGTGACCTCCGAGGGACATGCCAGCGCATGGCT
GAGGCTGTGCTGGGCACGCACGATGACTGGCAGATTGGCAAAACCAAGATCTTTCTGAAGGACCACCATGACATGTT
GCTGGAGGTGGAGCGGGACAAGGCCATCACAGACAGAGTCATTCTCCTCCAGAAGGTTATCCGGGGCTTCAAAGACA
GGTCCAACTTCCTGAGACTGAAGAGTGCTGCCACACTGATCCAGAGGCACTGGCGGGGCCACCACTGTAGGAAAAAC
TATGAGCTGATTCGTCTTGGCTTCCTGCGGCTGCAGGCCCTGCACCGCTCCCGGAAGCTGCACAAGCAGTACCGCCT
GGCCAGACAGCGCATAATAGAGTTCCAGGCCCGCTGCCGGGCCTATCTGGTGCGCAAGGCCTTCCGCCACCGCCTCT
GGGCCGTGATCACCGTGCAGGCCTATGCCCGAGGCATGATTGCCCGCCGGCTACACCGCCGCCTCCGGGTTGAGTAC
CAGCGGCGCCTCGAGGCAGAGAGGATGCGTCTGGCAGAGGAGGAGAAACTCCGAAAGGAGATGAGTGCCAAGAAGGC
CAAAGAGGAGGCTGAGCGCAAGCATCAGGAGCGCCTGGCTCAGCTAGCCCGCGAGGATGCGGAGCGGGAACTGAAGG
AGAAGGAGGAGGCTCGGAGGAAGAAGGAACTGCTGGAGCAGATGGAGAAGGCCCGCCACGAACCCATCAACCACTCA
GATATGGTGGACAAGATGTTTGGCTTCCTGGGGACTTCAGGCAGCCTGCCAGGCCAGGAAGGCCAGGCGCCTAGTGG
CTTTGAGGACCTAGAGCGCGGACGGAGGGAGATGGTGGAAGAGGATGTTGACGCTGCCCTGCCCCTGCCTGATGAAG
ACGAGGAGGACCTTTCTGAGTACAAATTCGCCAAGTTTGCTGCCACCTACTTCCAGGGCACAACCACACACTCCTAC
ACCCGGAGGCCTCTCAAGCAGCCGCTGCTCTACCACGACGATGAGGGTGACCAGCTGGCGGCGCTGGCTGTCTGGAT
CACCATCCTCCGGTTCATGGGGGACCTCCCAGAGCCCAAGTACCACACAGCCATGAGCGACGGCAGTGAGAAGATCC
CAGTGATGACTAAGATCTACGAGACCCTAGGCAAGAAGACATATAAGAGGGAGCTGCAGGCCTTGCAGGGCGAGGGC
GAGACCCAGCTCCCTGAGGGGCAGAAGAAGACCAGTGTGAGACACAAGTTGGTACACTTGACACTGAAGAAAAAGTC
CAAACTCACAGAAGAGGTGACCAAGAGGCTGAACGATGGGGAATCCACGGTACAGGGCAACAGCATGCTGGAGGATC
GGCCCACCTCAAATCTAGAGAAGCTGCACTTCATCATCGGCAACGGCATCCTGCGGCCTGCGCTGCGGGACGAGATT
TACTGCCAGATCAGTAAGCAGCTCACACACAACCCATCCAAGAGCAGCTATGCCAGGGGCTGGATCCTCGTGTCGCT
CTGTGTGGGCTGCTTCGCCCCCTCTGAGAAGTTCGTTAAGTACCTGCGGAACTTCATCCACGGAGGCCCACCTGGCT
ATGCTCCTTACTGTGAGGAGCGCCTGAGGAGGACCTTTGTCAACGGAACTCGGACACAGCCACCCAGCTGGCTGGAG
CTGCAGGCCACCAAGTCCAAGAAGCCCATCATGTTGCCCGTGACCTTCATGGATGGGACCACCAAGACCCTGCTAAC
AGATTCAGCAACTACAGCCAGGGAGCTGTGCAATGCTCTGGCTGACAAGATCTCACTCAAGGACCGCTTTGGCTTCT
CCCTCTACATCGCTCTGTTCGATAAGGTGTCCTCCCTGGGCAGCGGCAGTGACCATGTCATGGATGCCATCTCTCAG
TGTGAGCAGTACGCCAAGGAGCAGGGTGCTCAGGAGCGCAACGCCCCATGGAGGCTCTTCTTTAGAAAGGAGGTCTT
CACACCCTGGCACAACCCCTCGGAGGACAACGTGGCCACGAACCTCATCTACCAGCAGGTGGTGCGAGGAGTCAAGT
TTGGGGAGTACAGGTGTGAGAAGGAGGACGACCTGGCTGAGCTGGCTTCTCAGCAGTACTTTGTGGACTATGGTTCT
GAGATGATTCTGGAGCGCCTGCTGAGCCTCGTGCCCACTTACATCCCTGACCGTGAGATCACACCGCTGAAGAATCT
TGAGAAGTGGGCACAGCTGGCCATTGCTGCCCACAAGAAGGGAATTTATGCCCAGAGGAGAACTGACTCCCAGAAGG
TCAAAGAGGATGTGGTCAATTATGCCCGTTTCAAGTGGCCCTTGCTCTTCTCCAGGTTTTACGAAGCTTACAAATTC
TCAGGCCCTCCCCTCCCCAAGAGCGACGTCATCGTGGCTGTCAACTGGACGGGTGTGTACTTCGTGGACGAGCAGGA
GCAGGTGCTTCTGGAGCTGTCCTTCCCGGAGATCATGGCTGTGTCCAGCAGTAGGGAGTGCCGCGTCTTGCTCTCAC
TGGGCTGCTCTGACTTGGGCTGTGCTACTTGTCAATCGGGCCGGGCAGGGCTGACCCCGGCTGGACCCTGTTCTCCG
TGTTGGTCCTGTAGGGGAACAAAGATGATGGCCCCCAGCTTTACCCTGGCCACCATCAAAGGAGATGAGTACACCTT
CACATCCAGCAATGCTGAGGACATCCGTGACCTGGTGGTCACCTTTCTGGAGGGGCTACGGAAGAGGTCTAAGTATG
TGGTGGCACTGCAGGACAATCCTAACCCTGCTGGTGAGGAGTCAGGCTTCCTCAGCTTCGCCAAGGGAGACCTCATC
ATCCTTGACCATGATACTGGTGAGCAGGTCATGAACTCAGGCTGGGCCAACGGCATCAACGAGAGGACCAAGCAGCG
CGGCGACTTCCCCACTGACTGTGTATACGTCATGCCCACTGTCACCTTGCCACCAAGGGAGATTGTGGCCCTGGTCA
CTATGACCCCAGACCAGAGGCAGGATGTCGTCCGGCTCCTGCAGCTTCGCACAGCAGAGCCAGAGGTGCGCGCCAAG
CCCTACACGCTAGAGGAGTTCTCCTACGACTACTTCAGGCCCCCACCCAAGCACACGCTGAGCCGTGTCATGGTGTC
CAAGGCCCGCGGTAAGGACAGGCTGTGGAGCCACACACGAGAGCCCCTCAAGCAGGCCCTGCTCAAGAAGATCCTGG
GCAGTGAAGAACTCTCCCAGGAAGCCTGCATGGCCTTTGTAGCTGTGCTCAAGTACATGGGCGACTACCCATCCAAG
AGGATGCGATCCGTCAATGAGCTCACTGACCAGATCTTTGAGTGGGCACTCAAGGCTGAGCCCCTCAAGGATGAGGC
CTACGTGCAGATCCTGAAGCAGCTGACTGACAATCACATCAGGTACAGCGAAGAGAGGGGCTGGGAACTGCTGTGGC
TGTGCACGGGCCTCTTCCCGCCCAGCAACATCCTCCTGCCTCATGTTCAGCGGTTTCTGCAGTCCCGCAAGCACTGT
CCTCTTGCCATTGACTGCCTGCAGAGGCTCCAGAAAGCCCTGAGAAATGGCTCCCGGAAGTACCCTCCGCACCTGGT
GGAGGTGGAGGCCATCCAACATAAGACTACCCAGATCTTCCACAAGGTCTACTTCCCCGATGACACGGACGAGGCTT
TTGAGGTGGAGTCCAGCACCAAGGCCAAGGACTTCTGCCAGAACATCGCCAGCCGGCTGCTGCTCAAGTCTTCCGAG
GGATTCAGCCTTTTTGTCAAAATCGCAGATAAGGTCATCAGCGTCCCAGAGAATGATTTCTTCTTTGACTTTGTCCG
ACACCTGACAGACTGGATAAAGAAAGCACGGCCCATCAAGGACGGAATCGTGCCCTCACTAACCTACCAGGTGTTCT
TCATGAAGAAGCTGTGGACCACCACAGTGCCGGGCAAGGACCCCATGGCTGACTCCATCTTCCACTATTACCAGGAA
CTGCCCAAATATCTCCGAGGCTACCACAAGTGCACCCGGGAGGAGGTGCTGCAGCTGGGCGCACTCATCTACAGGGT
CAAGTTTGAGGAGGACAAATCCTACTTCCCTAGCATCCCCAAGTTGCTGAGGGAGCTGGTACCCCAGGACCTAATCC
GGCAGGTCTCACCTGATGACTGGAAACGGTCTATTGTCGCCTACTTCAACAAACATGCGGGGAAGTCCAAGGAGGAA
GCCAAGCTGGCCTTCCTCAAACTCATCTTCAAGTGGCCCACCTTTGGCTCAGCCTTCTTTGAGGTGAAGCAAACTAC
AGAACCAAACTTCCCAGAGATTCTCTTAATTGCCATCAACAAGTACGGGGTCAGCCTCATCGATCCCAGAACCAAGG
ACATCCTGACTACTCACCCCTTCACCAAGATCTCCAACTGGAGTAGTGGCAACACCTACTTCCACATCACCATTGGG
AACTTGGTCCGTGGGAGCAAACTGCTCTGTGAGACATCGCTGGGATACAAAATGGATGATCTTCTGACTTCCTACAT
CAGCCAGATGCTCACAGCCATGAGCAAGCAGAGGAACTCCAGGAGTGGAAGGTGAACCTCAGAGGAGACGCTGGCTC
AGGCCTTGGCCCTCTAGGCAGGGAAGCTGGACTGACCATATCTGCTGGGCATCTGATCTGCCTGCCACGAGGTCCAG
ACTCTTCTGCATCCACCTATGGCATCTGGGTTTGCTGTCACCCTACTTTGTTGTGGCCTTCCTGGTGTAAGAGTCTG
TGTTCCTTGGTCACCTCTCCTGATTCAGACCAGCTCCATCAAGCAACCTCTTTTGACTTTCTGTATATGGATGGCAC
AGAGGAATCAAGGACAACTTAGCTCTCTGCATACTTGGAACAACCAAACTATTTGTACATTGAACGGATGCTCTGAA
ACCCAAGGGACTGGGCTCAGGGTCCTCAGCACTGGCCCCTGTCATAAGCACTACCACTAAGGACTCTCTGGAGGACT
CCTCAGTATCATCTGCTCCAGGAAGCCCCCTAGACTACCTCCTGAGTCTGGACAAAGCCTCCTGATTCTACCTGGAT
CACTCCTGTTATGTGACAGTTATGTGGTGGGTCCCTGCTAAAATCTCCCTGACCACCTGAGGGCATAAAGCATGTGT
CTTATTCTCTGG (SEQ ID NO: 7)
Mus musculus myosin VIIA (Myo7a), isoform 1, protein
NCBI RefSeq NP_001243010.1
MVILQKGDYVWMDLKSGQEFDVPIGAVVKLCDSGQIQVVDDEDNEHWISPQNATHIKPMHPTSVHGVEDMIRLGDLN
EAGILRNLLIRYRDHLIYTYTGSILVAVNPYQLLSIYSPEHIRQYTNKKIGEMPPHIFAIADNCYFNMKRNNRDQCC
IISGESGAGKTESTKLILQFLAAISGQHSWIEQQVLEATPILEAFGNAKTIRNDNSSRFGKYIDIHFNKRGAIEGAK
IEQYLLEKSRVCRQAPDERNYHVFYCMLEGMNEEEKKKLGLGQAADYNYLAMGNCITCEGRVDSQEYANIRSAMKVL
MFTDTENWEISKLLAAILHMGNLQYEARTFENLDACEVLFSPSLATAASLLEVNPPDLMSCLTSRTLITRGETVSTP
LSREQALDVRDAFVKGIYGRLFVWIVEKINAAIYKPPPLEVKNSRRSIGLLDIFGFENFTVNSFEQLCINFANEHLQ
QFFVRHVFKLEQEEYDLESIDWLHIEFTDNQEALDMIANRPMNVISLIDEESKFPKGTDATMLHKLNSQHKLNANYV
PPKNSHETQFGINHFAGVVYYESQGFLEKNRDTLHGDIIQLVHSSRNKFIKQIFQADVAMGAETRKRSPTLSSQFKR
SLELLMRTLGACQPFFVRCIKPNEFKKPMLFDRHLCVRQLRYSGMMETIRIRHAGYPIRYSFVEFVERYRVLLPGVK
PAYKQGDLRGTCQRMAEAVLGTHDDWQIGKTKIFLKDHHDMLLEVERDKAITDRVILLQKVIRGFKDRSNFLRLKSA
ATLIQRHWRGHHCRKNYELIRLGFLRLQALHRSRKLHKQYRLARQRIIEFQARCRAYLVRKAFRHRLWAVITVQAYA
RGMIARRLHRRLRVEYQRRLEAERMRLAEEEKLRKEMSAKKAKEEAERKHQERLAQLAREDAERELKEKEEARRKKE
LLEQMEKARHEPINHSDMVDKMFGFLGTSGSLPGQEGQAPSGFEDLERGRREMVEEDVDAALPLPDEDEEDLSEYKF
AKFAATYFQGTTTHSYTRRPLKQPLLYHDDEGDQLAALAVWITILRFMGDLPEPKYHTAMSDGSEKIPVMTKIYETL
GKKTYKRELQALQGEGETQLPEGQKKTSVRHKLVHLTLKKKSKLTEEVTKRLNDGESTVQGNSMLEDRPTSNLEKLH
FIIGNGILRPALRDEIYCQISKQLTHNPSKSSYARGWILVSLCVGCFAPSEKFVKYLRNFIHGGPPGYAPYCEERLR
RTFVNGTRTQPPSWLELQATKSKKPIMLPVTFMDGTTKTLLTDSATTARELCNALADKISLKDRFGFSLYIALFDKV
SSLGSGSDHVMDAISQCEQYAKEQGAQERNAPWRLFFRKEVFTPWHNPSEDNVATNLIYQQVVRGVKFGEYRCEKED
DLAELASQQYFVDYGSEMILERLLSLVPTYIPDREITPLKNLEKWAQLAIAAHKKGIYAQRRTDSQKVKEDVVNYAR
FKWPLLFSRFYEAYKFSGPPLPKSDVIVAVNWTGVYFVDEQEQVLLELSFPEIMAVSSSRECRVLLSLGCSDLGCAT
CQSGRAGLTPAGPCSPCWSCRGTKMMAPSFTLATIKGDEYTFTSSNAEDIRDLVVTFLEGLRKRSKYVVALQDNPNP
AGEESGFLSFAKGDLIILDHDTGEQVMNSGWANGINERTKQRGDFPTDCVYVMPTVTLPPREIVALVTMTPDQRQDV
VRLLQLRTAEPEVRAKPYTLEEFSYDYFRPPPKHTLSRVMVSKARGKDRLWSHTREPLKQALLKKILGSEELSQEAC
MAFVAVLKYMGDYPSKRMRSVNELTDQIFEWALKAEPLKDEAYVQILKQLTDNHIRYSEERGWELLWLCTGLFPPSN
ILLPHVQRFLQSRKHCPLAIDCLQRLQKALRNGSRKYPPHLVEVEAIQHKTTQIFHKVYFPDDTDEAFEVESSTKAK
DFCQNIASRLLLKSSEGFSLFVKIADKVISVPENDFFFDFVRHLTDWIKKARPIKDGIVPSLTYQVFFMKKLWTTTV
PGKDPMADSIFHYYQELPKYLRGYHKCTREEVLQLGALIYRVKFEEDKSYFPSIPKLLRELVPQDLIRQVSPDDWKR
SIVAYFNKHAGKSKEEAKLAFLKLIFKWPTFGSAFFEVKQTTEPNFPEILLIAINKYGVSLIDPRIKDILTTHPFTK
ISNWSSGNTYFHITIGNLVRGSKLLCETSLGYKMDDLLTSYISQMLTAMSKQRNSRSGR (SEQ ID NO: 8)
Mus musculus myosin VIIA (Myo7a), transcript variant 3, mRNA
NCBI RefSeq NM_001256082.1
GGCGCTAGGCTGTGAGATCATCAGAGGACTCCGTTACCCACACAGCTGCCAGAGGGGAGTTGTCAAAGTGAAACCCC
AGGGACTGGGGGTGTAGTTTGTGGGGTCAGGGGGACTATGTATGGATGGACCTGAAGTCAGGCCAGGAGTTTGATGT
GCCCATCGGGGCCGTGGTGAAGCTCTGCGACTCGGGCCAGATCCAGGTGGTGGATGATGAAGACAATGAACACTGGA
TATCCCCTCAGAATGCCACGCACATCAAGCCAATGCACCCCACATCGGTGCACGGCGTGGAGGACATGATCCGCCTG
GGGGATCTCAACGAGGCAGGCATCCTTCGAAACCTTCTCATTCGCTACCGGGACCACCTCATCTATACCAGCTGTGG
AGGACGGACGTACACAGGTTCCATCCTGGTGGCCGTGAACCCCTACCAGCTGCTCTCCATCTACTCGCCAGAGCACA
TCCGCCAGTACACCAACAAGAAGATAGGGGAGATGCCCCCCCACATCTTCGCCATTGCTGACAACTGCTACTTCAAC
ATGAAACGCAACAACCGGGACCAGTGCTGTATTATCAGCGGGGAGTCGGGAGCTGGCAAGACAGAGAGCACAAAGTT
GATCCTGCAGTTCCTGGCAGCCATCAGTGGACAGCACTCATGGATCGAGCAGCAGGTGCTGGAGGCCACCCCGATCC
TGGAAGCATTTGGGAACGCCAAGACCATCCGCAACGACAACTCTAGCCGCTTTGGCAAGTACATTGACATCCACTTT
AACAAGCGTGGTGCCATCGAGGGCGCCAAAATAGAGCAATACCTGCTGGAGAAGTCACGTGTCTGCCGCCAGGCCCC
TGACGAGAGGAACTATCACGTGTTCTACTGTATGCTGGAGGGCATGAATGAGGAGGAGAAGAAGAAACTGGGCCTAG
GCCAGGCCGCTGACTACAACTACTTGGCCATGGGTAACTGCATCACCTGTGAGGGCCGCGTGGACAGTCAGGAGTAT
GCCAACATCCGCTCTGCCATGAAGGTTCTCATGTTCACAGACACGGAGAACTGGGAGATCTCGAAGCTTCTGGCTGC
CATCCTACACATGGGCAATCTGCAGTATGAGGCCCGGACATTTGAGAACTTGGATGCGTGTGAAGTCCTCTTCTCCC
CATCGCTGGCCACGGCAGCTTCTCTGCTCGAGGTGAACCCCCCAGACCTGATGAGCTGCCTCACCAGCCGCACCCTC
ATCACCCGTGGGGAGACGGTGTCCACCCCTCTCAGCAGGGAACAGGCGCTGGATGTGCGAGATGCCTTTGTCAAGGG
CATCTATGGGCGGCTCTTTGTGTGGATTGTGGAGAAGATCAACGCAGCAATCTACAAGCCACCCCCCCTGGAAGTGA
AGAACTCTCGCCGGTCCATCGGTCTCCTGGACATCTTTGGATTTGAGAACTTCACTGTGAACAGCTTCGAGCAGCTC
TGCATTAACTTTGCCAATGAGCACCTGCAGCAATTCTTCGTGCGGCACGTGTTCAAGCTGGAGCAGGAGGAGTACGA
CCTGGAGAGCATCGACTGGTTGCACATTGAGTTCACTGACAACCAGGAAGCACTGGACATGATTGCCAACCGGCCTA
TGAACGTCATCTCCCTCATCGATGAGGAGAGCAAGTTCCCCAAGGGCACGGATGCCACCATGCTGCATAAGCTGAAC
TCACAGCACAAGCTCAATGCCAACTACGTGCCACCCAAGAACAGCCACGAGACCCAGTTTGGAATCAACCACTTTGC
GGGTGTTGTCTATTATGAGAGTCAAGGCTTCCTGGAGAAGAACCGAGACACCCTGCATGGGGACATCATCCAGCTGG
TCCACTCTTCCCGGAACAAGTTCATAAAGCAGATTTTCCAAGCTGACGTTGCCATGGGTGCCGAGACCAGGAAGCGC
TCGCCTACACTCAGCAGCCAGTTCAAGCGGTCTCTGGAGCTGCTGATGCGCACACTGGGCGCCTGCCAGCCCTTCTT
TGTGCGTTGTATCAAACCCAATGAGTTCAAGAAGCCCATGCTCTTCGACCGGCACTTGTGTGTACGCCAGCTGCGAT
ATTCGGGCATGATGGAGACAATCCGCATCCGCCACGCAGGCTACCCCATTCGCTACAGCTTTGTGGAGTTTGTGGAG
CGCTACCGGGTACTGCTGCCTGGTGTGAAGCCAGCATACAAGCAGGGTGACCTCCGAGGGACATGCCAGCGCATGGC
TGAGGCTGTGCTGGGCACGCACGATGACTGGCAGATTGGCAAAACCAAGATCTTTCTGAAGGACCACCATGACATGT
TGCTGGAGGTGGAGCGGGACAAGGCCATCACAGACAGAGTCATTCTCCTCCAGAAGGTTATCCGGGGCTTCAAAGAC
AGGTCCAACTTCCTGAGACTGAAGAGTGCTGCCACACTGATCCAGAGGCACTGGCGGGGCCACCACTGTAGGAAAAA
CTATGAGCTGATTCGTCTTGGCTTCCTGCGGCTGCAGGCCCTGCACCGCTCCCGGAAGCTGCACAAGCAGTACCGCC
TGGCCAGACAGCGCATAATAGAGTTCCAGGCCCGCTGCCGGGCCTATCTGGTGCGCAAGGCCTTCCGCCACCGCCTC
TGGGCCGTGATCACCGTGCAGGCCTATGCCCGAGGCATGATTGCCCGCCGGCTACACCGCCGCCTCCGGGTTGAGTA
CCAGCGGCGCCTCGAGGCAGAGAGGATGCGTCTGGCAGAGGAGGAGAAACTCCGAAAGGAGATGAGTGCCAAGAAGG
CCAAAGAGGAGGCTGAGCGCAAGCATCAGGAGCGCCTGGCTCAGCTAGCCCGCGAGGATGCGGAGCGGGAACTGAAG
GAGAAGGAGGAGGCTCGGAGGAAGAAGGAACTGCTGGAGCAGATGGAGAAGGCCCGCCACGAACCCATCAACCACTC
AGATATGGTGGACAAGATGTTTGGCTTCCTGGGGACTTCAGGCAGCCTGCCAGGCCAGGAAGGCCAGGCGCCTAGTG
GCTTTGAGGACCTAGAGCGCGGACGGAGGGAGATGGTGGAAGAGGATGTTGACGCTGCCCTGCCCCTGCCTGATGAA
GACGAGGAGGACCTTTCTGAGTACAAATTCGCCAAGTTTGCTGCCACCTACTTCCAGGGCACAACCACACACTCCTA
CACCCGGAGGCCTCTCAAGCAGCCGCTGCTCTACCACGACGATGAGGGTGACCAGCTGGCGGCGCTGGCTGTCTGGA
TCACCATCCTCCGGTTCATGGGGGACCTCCCAGAGCCCAAGTACCACACAGCCATGAGCGACGGCAGTGAGAAGATC
CCAGTGATGACTAAGATCTACGAGACCCTAGGCAAGAAGACATATAAGAGGGAGCTGCAGGCCTTGCAGGGCGAGGG
CGAGACCCAGCTCCCTGAGGGGCAGAAGAAGACCAGTGTGAGACACAAGTTGGTACACTTGACACTGAAGAAAAAGT
CCAAACTCACAGAAGAGGTGACCAAGAGGCTGAACGATGGGGAATCCACGGTACAGGGCAACAGCATGCTGGAGGAT
CGGCCCACCTCAAATCTAGAGAAGCTGCACTTCATCATCGGCAACGGCATCCTGCGGCCTGCGCTGCGGGACGAGAT
TTACTGCCAGATCAGTAAGCAGCTCACACACAACCCATCCAAGAGCAGCTATGCCAGGGGCTGGATCCTCGTGTCGC
TCTGTGTGGGCTGCTTCGCCCCCTCTGAGAAGTTCGTTAAGTACCTGCGGAACTTCATCCACGGAGGCCCACCTGGC
TATGCTCCTTACTGTGAGGAGCGCCTGAGGAGGACCTTTGTCAACGGAACTCGGACACAGCCACCCAGCTGGCTGGA
GCTGCAGGCCACCAAGTCCAAGAAGCCCATCATGTTGCCCGTGACCTTCATGGATGGGACCACCAAGACCCTGCTAA
CAGATTCAGCAACTACAGCCAGGGAGCTGTGCAATGCTCTGGCTGACAAGATCTCACTCAAGGACCGCTTTGGCTTC
TCCCTCTACATCGCTCTGTTCGATAAGGTGTCCTCCCTGGGCAGCGGCAGTGACCATGTCATGGATGCCATCTCTCA
GTGTGAGCAGTACGCCAAGGAGCAGGGTGCTCAGGAGCGCAACGCCCCATGGAGGCTCTTCTTTAGAAAGGAGGTCT
TCACACCCTGGCACAACCCCTCGGAGGACAACGTGGCCACGAACCTCATCTACCAGCAGGTGGTGCGAGGAGTCAAG
TTTGGGGAGTACAGGTGTGAGAAGGAGGACGACCTGGCTGAGCTGGCTTCTCAGCAGTACTTTGTGGACTATGGTTC
TGAGATGATTCTGGAGCGCCTGCTGAGCCTCGTGCCCACTTACATCCCTGACCGTGAGATCACACCGCTGAAGAATC
TTGAGAAGTGGGCACAGCTGGCCATTGCTGCCCACAAGAAGGGAATTTATGCCCAGAGGAGAACTGACTCCCAGAAG
GTCAAAGAGGATGTGGTCAATTATGCCCGTTTCAAGTGGCCCTTGCTCTTCTCCAGGTTTTACGAAGCTTACAAATT
CTCAGGCCCTCCCCTCCCCAAGAGCGACGTCATCGTGGCTGTCAACTGGACGGGTGTGTACTTCGTGGACGAGCAGG
AGCAGGTGCTTCTGGAGCTGTCCTTCCCGGAGATCATGGCTGTGTCCAGCAGTAGGGGAACAAAGATGATGGCCCCC
AGCTTTACCCTGGCCACCATCAAAGGAGATGAGTACACCTTCACATCCAGCAATGCTGAGGACATCCGTGACCTGGT
GGTCACCTTTCTGGAGGGGCTACGGAAGAGGTCTAAGTATGTGGTGGCACTGCAGGACAATCCTAACCCTGCTGGTG
AGGAGTCAGGCTTCCTCAGCTTCGCCAAGGGAGACCTCATCATCCTTGACCATGATACTGGTGAGCAGGTCATGAAC
TCAGGCTGGGCCAACGGCATCAACGAGAGGACCAAGCAGCGCGGCGACTTCCCCACTGACTGTGTATACGTCATGCC
CACTGTCACCTTGCCACCAAGGGAGATTGTGGCCCTGGTCACTATGACCCCAGACCAGAGGCAGGATGTCGTCCGGC
TCCTGCAGCTTCGCACAGCAGAGCCAGAGGTGCGCGCCAAGCCCTACACGCTAGAGGAGTTCTCCTACGACTACTTC
AGGCCCCCACCCAAGCACACGCTGAGCCGTGTCATGGTGTCCAAGGCCCGCGGTAAGGACAGGCTGTGGAGCCACAC
ACGAGAGCCCCTCAAGCAGGCCCTGCTCAAGAAGATCCTGGGCAGTGAAGAACTCTCCCAGGAAGCCTGCATGGCCT
TTGTAGCTGTGCTCAAGTACATGGGCGACTACCCATCCAAGAGGATGCGATCCGTCAATGAGCTCACTGACCAGATC
TTTGAGTGGGCACTCAAGGCTGAGCCCCTCAAGGATGAGGCCTACGTGCAGATCCTGAAGCAGCTGACTGACAATCA
CATCAGGTACAGCGAAGAGAGGGGCTGGGAACTGCTGTGGCTGTGCACGGGCCTCTTCCCGCCCAGCAACATCCTCC
TGCCTCATGTTCAGCGGTTTCTGCAGTCCCGCAAGCACTGTCCTCTTGCCATTGACTGCCTGCAGAGGCTCCAGAAA
GCCCTGAGAAATGGCTCCCGGAAGTACCCTCCGCACCTGGTGGAGGTGGAGGCCATCCAACATAAGACTACCCAGAT
CTTCCACAAGGTCTACTTCCCCGATGACACGGACGAGGCTTTTGAGGTGGAGTCCAGCACCAAGGCCAAGGACTTCT
GCCAGAACATCGCCAGCCGGCTGCTGCTCAAGTCTTCCGAGGGATTCAGCCTTTTTGTCAAAATCGCAGATAAGGTC
ATCAGCGTCCCAGAGAATGATTTCTTCTTTGACTTTGTCCGACACCTGACAGACTGGATAAAGAAAGCACGGCCCAT
CAAGGACGGAATCGTGCCCTCACTAACCTACCAGGTGTTCTTCATGAAGAAGCTGTGGACCACCACAGTGCCGGGCA
AGGACCCCATGGCTGACTCCATCTTCCACTATTACCAGGAACTGCCCAAATATCTCCGAGGCTACCACAAGTGCACC
CGGGAGGAGGTGCTGCAGCTGGGCGCACTCATCTACAGGGTCAAGTTTGAGGAGGACAAATCCTACTTCCCTAGCAT
CCCCAAGTTGCTGAGGGAGCTGGTACCCCAGGACCTAATCCGGCAGGTCTCACCTGATGACTGGAAACGGTCTATTG
TCGCCTACTTCAACAAACATGCGGGGAAGTCCAAGGAGGAAGCCAAGCTGGCCTTCCTCAAACTCATCTTCAAGTGG
CCCACCTTTGGCTCAGCCTTCTTTGAGGTGAAGCAAACTACAGAACCAAACTTCCCAGAGATTCTCTTAATTGCCAT
CAACAAGTACGGGGTCAGCCTCATCGATCCCAGAACCAAGGACATCCTGACTACTCACCCCTTCACCAAGATCTCCA
ACTGGAGTAGTGGCAACACCTACTTCCACATCACCATTGGGAACTTGGTCCGTGGGAGCAAACTGCTCTGTGAGACA
TCGCTGGGATACAAAATGGATGATCTTCTGACTTCCTACATCAGCCAGATGCTCACAGCCATGAGCAAGCAGAGGAA
CTCCAGGAGTGGAAGGTGAACCTCAGAGGAGACGCTGGCTCAGGCCTTGGCCCTCTAGGCAGGGAAGCTGGACTGAC
CATATCTGCTGGGCATCTGATCTGCCTGCCACGAGGTCCAGACTCTTCTGCATCCACCTATGGCATCTGGGTTTGCT
GTCACCCTACTTTGTTGTGGCCTTCCTGGTGTAAGAGTCTGTGTTCCTTGGTCACCTCTCCTGATTCAGACCAGCTC
CATCAAGCAACCTCTTTTGACTTTCTGTATATGGATGGCACAGAGGAATCAAGGACAACTTAGCTCTCTGCATACTT
GGAACAACCAAACTATTTGTACATTGAACGGATGCTCTGAAACCCAAGGGACTGGGCTCAGGGTCCTCAGCACTGGC
CCCTGTCATAAGCACTACCACTAAGGACTCTCTGGAGGACTCCTCAGTATCATCTGCTCCAGGAAGCCCCCTAGACT
ACCTCCTGAGTCTGGACAAAGCCTCCTGATTCTACCTGGATCACTCCTGTTATGTGACAGTTATGTGGTGGGTCCCT
GCTAAAATCTCCCTGACCACCTGAGGGCATAAAGCATGTGTCTTATTCTCTGG (SEQ ID NO: 9)
Mus musculus myosin VIIA (Myo7a), isoform 3, protein
NCBI RefSeq NP_001243011.1
MDLKSGQEFDVPIGAVVKLCDSGQIQVVDDEDNEHWISPQNATHIKPMHPTSVHGVEDMIRLGDLNEAGILRNLLIR
YRDHLIYTSCGGRTYTGSILVAVNPYQLLSIYSPEHIRQYTNKKIGEMPPHIFAIADNCYFNMKRNNRDQCCIISGE
SGAGKTESTKLILQFLAAISGQHSWIEQQVLEATPILEAFGNAKTIRNDNSSRFGKYIDIHFNKRGAIEGAKIEQYL
LEKSRVCRQAPDERNYHVFYCMLEGMNEEEKKKLGLGQAADYNYLAMGNCITCEGRVDSQEYANIRSAMKVLMFTDT
ENWEISKLLAAILHMGNLQYEARTFENLDACEVLFSPSLATAASLLEVNPPDLMSCLTSRTLITRGETVSTPLSREQ
ALDVRDAFVKGIYGRLFVWIVEKINAAIYKPPPLEVKNSRRSIGLLDIFGFENFTVNSFEQLCINFANEHLQQFFVR
HVFKLEQEEYDLESIDWLHIEFTDNQEALDMIANRPMNVISLIDEESKFPKGTDATMLHKLNSQHKLNANYVPPKNS
HETQFGINHFAGVVYYESQGFLEKNRDTLHGDIIQLVHSSRNKFIKQIFQADVAMGAETRKRSPTLSSQFKRSLELL
MRTLGACQPFFVRCIKPNEFKKPMLFDRHLCVRQLRYSGMMETIRIRHAGYPIRYSFVEFVERYRVLLPGVKPAYKQ
GDLRGTCQRMAEAVLGTHDDWQIGKTKIFLKDHHDMLLEVERDKAITDRVILLQKVIRGFKDRSNFLRLKSAATLIQ
RHWRGHHCRKNYELIRLGFLRLQALHRSRKLHKQYRLARQRIIEFQARCRAYLVRKAFRHRLWAVITVQAYARGMIA
RRLHRRLRVEYQRRLEAERMRLAEEEKLRKEMSAKKAKEEAERKHQERLAQLAREDAERELKEKEEARRKKELLEQM
EKARHEPINHSDMVDKMFGFLGTSGSLPGQEGQAPSGFEDLERGRREMVEEDVDAALPLPDEDEEDLSEYKFAKFAA
TYFQGTTTHSYTRRPLKQPLLYHDDEGDQLAALAVWITILRFMGDLPEPKYHTAMSDGSEKIPVMTKIYETLGKKTY
KRELQALQGEGETQLPEGQKKTSVRHKLVHLTLKKKSKLTEEVTKRLNDGESTVQGNSMLEDRPTSNLEKLHFIIGN
GILRPALRDEIYCQISKQLTHNPSKSSYARGWILVSLCVGCFAPSEKFVKYLRNFIHGGPPGYAPYCEERLRRTFVN
GTRTQPPSWLELQATKSKKPIMLPVTFMDGTTKTLLTDSATTARELCNALADKISLKDRFGFSLYIALFDKVSSLGS
GSDHVMDAISQCEQYAKEQGAQERNAPWRLFFRKEVFTPWHNPSEDNVATNLIYQQVVRGVKFGEYRCEKEDDLAEL
ASQQYFVDYGSEMILERLLSLVPTYIPDREITPLKNLEKWAQLAIAAHKKGIYAQRRTDSQKVKEDVVNYARFKWPL
LFSRFYEAYKFSGPPLPKSDVIVAVNWTGVYFVDEQEQVLLELSFPEIMAVSSSRGTKMMAPSFTLATIKGDEYTFT
SSNAEDIRDLVVTFLEGLRKRSKYVVALQDNPNPAGEESGFLSFAKGDLIILDHDTGEQVMNSGWANGINERTKQRG
DFPTDCVYVMPTVTLPPREIVALVTMTPDQRQDVVRLLQLRTAEPEVRAKPYTLEEFSYDYFRPPPKHTLSRVMVSK
ARGKDRLWSHTREPLKQALLKKILGSEELSQEACMAFVAVLKYMGDYPSKRMRSVNELTDQIFEWALKAEPLKDEAY
VQILKQLTDNHIRYSEERGWELLWLCTGLFPPSNILLPHVQRFLQSRKHCPLAIDCLQRLQKALRNGSRKYPPHLVE
VEAIQHKTTQIFHKVYFPDDTDEAFEVESSTKAKDFCQNIASRLLLKSSEGFSLFVKIADKVISVPENDFFFDFVRH
LTDWIKKARPIKDGIVPSLTYQVFFMKKLWTTTVPGKDPMADSIFHYYQELPKYLRGYHKCTREEVLQLGALIYRVK
FEEDKSYFPSIPKLLRELVPQDLIRQVSPDDWKRSIVAYFNKHAGKSKEEAKLAFLKLIFKWPTFGSAFFEVKQTTE
PNFPEILLIAINKYGVSLIDPRIKDILTTHPFTKISNWSSGNTYFHITIGNLVRGSKLLCETSLGYKMDDLLTSYIS
QMLTAMSKQRNSRSGR (SEQ ID NO: 10)
Mus musculus myosin VIIA (Myo7a), transcript variant 4, mRNA
NCBI RefSeq NM_001256083.1
GGCGCTAGGCTGTGAGATCATCAGAGGACTCCGTTACCCACACAGCTGCCAGAGGGGAGTTGTCAAAGTGAAACCCC
AGGGACTGGGGGTGTAGTTTGTGGGGTCAGGGGGACTATGTATGGATGGACCTGAAGTCAGGCCAGGAGTTTGATGT
GCCCATCGGGGCCGTGGTGAAGCTCTGCGACTCGGGCCAGATCCAGGTGGTGGATGATGAAGACAATGAACACTGGA
TATCCCCTCAGAATGCCACGCACATCAAGCCAATGCACCCCACATCGGTGCACGGCGTGGAGGACATGATCCGCCTG
GGGGATCTCAACGAGGCAGGCATCCTTCGAAACCTTCTCATTCGCTACCGGGACCACCTCATCTATACGTACACAGG
TTCCATCCTGGTGGCCGTGAACCCCTACCAGCTGCTCTCCATCTACTCGCCAGAGCACATCCGCCAGTACACCAACA
AGAAGATAGGGGAGATGCCCCCCCACATCTTCGCCATTGCTGACAACTGCTACTTCAACATGAAACGCAACAACCGG
GACCAGTGCTGTATTATCAGCGGGGAGTCGGGAGCTGGCAAGACAGAGAGCACAAAGTTGATCCTGCAGTTCCTGGC
AGCCATCAGTGGACAGCACTCATGGATCGAGCAGCAGGTGCTGGAGGCCACCCCGATCCTGGAAGCATTTGGGAACG
CCAAGACCATCCGCAACGACAACTCTAGCCGCTTTGGCAAGTACATTGACATCCACTTTAACAAGCGTGGTGCCATC
GAGGGCGCCAAAATAGAGCAATACCTGCTGGAGAAGTCACGTGTCTGCCGCCAGGCCCCTGACGAGAGGAACTATCA
CGTGTTCTACTGTATGCTGGAGGGCATGAATGAGGAGGAGAAGAAGAAACTGGGCCTAGGCCAGGCCGCTGACTACA
ACTACTTGGCCATGGGTAACTGCATCACCTGTGAGGGCCGCGTGGACAGTCAGGAGTATGCCAACATCCGCTCTGCC
ATGAAGGTTCTCATGTTCACAGACACGGAGAACTGGGAGATCTCGAAGCTTCTGGCTGCCATCCTACACATGGGCAA
TCTGCAGTATGAGGCCCGGACATTTGAGAACTTGGATGCGTGTGAAGTCCTCTTCTCCCCATCGCTGGCCACGGCAG
CTTCTCTGCTCGAGGTGAACCCCCCAGACCTGATGAGCTGCCTCACCAGCCGCACCCTCATCACCCGTGGGGAGACG
GTGTCCACCCCTCTCAGCAGGGAACAGGCGCTGGATGTGCGAGATGCCTTTGTCAAGGGCATCTATGGGCGGCTCTT
TGTGTGGATTGTGGAGAAGATCAACGCAGCAATCTACAAGCCACCCCCCCTGGAAGTGAAGAACTCTCGCCGGTCCA
TCGGTCTCCTGGACATCTTTGGATTTGAGAACTTCACTGTGAACAGCTTCGAGCAGCTCTGCATTAACTTTGCCAAT
GAGCACCTGCAGCAATTCTTCGTGCGGCACGTGTTCAAGCTGGAGCAGGAGGAGTACGACCTGGAGAGCATCGACTG
GTTGCACATTGAGTTCACTGACAACCAGGAAGCACTGGACATGATTGCCAACCGGCCTATGAACGTCATCTCCCTCA
TCGATGAGGAGAGCAAGTTCCCCAAGGGCACGGATGCCACCATGCTGCATAAGCTGAACTCACAGCACAAGCTCAAT
GCCAACTACGTGCCACCCAAGAACAGCCACGAGACCCAGTTTGGAATCAACCACTTTGCGGGTGTTGTCTATTATGA
GAGTCAAGGCTTCCTGGAGAAGAACCGAGACACCCTGCATGGGGACATCATCCAGCTGGTCCACTCTTCCCGGAACA
AGTTCATAAAGCAGATTTTCCAAGCTGACGTTGCCATGGGTGCCGAGACCAGGAAGCGCTCGCCTACACTCAGCAGC
CAGTTCAAGCGGTCTCTGGAGCTGCTGATGCGCACACTGGGCGCCTGCCAGCCCTTCTTTGTGCGTTGTATCAAACC
CAATGAGTTCAAGAAGCCCATGCTCTTCGACCGGCACTTGTGTGTACGCCAGCTGCGATATTCGGGCATGATGGAGA
CAATCCGCATCCGCCACGCAGGCTACCCCATTCGCTACAGCTTTGTGGAGTTTGTGGAGCGCTACCGGGTACTGCTG
CCTGGTGTGAAGCCAGCATACAAGCAGGGTGACCTCCGAGGGACATGCCAGCGCATGGCTGAGGCTGTGCTGGGCAC
GCACGATGACTGGCAGATTGGCAAAACCAAGATCTTTCTGAAGGACCACCATGACATGTTGCTGGAGGTGGAGCGGG
ACAAGGCCATCACAGACAGAGTCATTCTCCTCCAGAAGGTTATCCGGGGCTTCAAAGACAGGTCCAACTTCCTGAGA
CTGAAGAGTGCTGCCACACTGATCCAGAGGCACTGGCGGGGCCACCACTGTAGGAAAAACTATGAGCTGATTCGTCT
TGGCTTCCTGCGGCTGCAGGCCCTGCACCGCTCCCGGAAGCTGCACAAGCAGTACCGCCTGGCCAGACAGCGCATAA
TAGAGTTCCAGGCCCGCTGCCGGGCCTATCTGGTGCGCAAGGCCTTCCGCCACCGCCTCTGGGCCGTGATCACCGTG
CAGGCCTATGCCCGAGGCATGATTGCCCGCCGGCTACACCGCCGCCTCCGGGTTGAGTACCAGCGGCGCCTCGAGGC
AGAGAGGATGCGTCTGGCAGAGGAGGAGAAACTCCGAAAGGAGATGAGTGCCAAGAAGGCCAAAGAGGAGGCTGAGC
GCAAGCATCAGGAGCGCCTGGCTCAGCTAGCCCGCGAGGATGCGGAGCGGGAACTGAAGGAGAAGGAGGAGGCTCGG
AGGAAGAAGGAACTGCTGGAGCAGATGGAGAAGGCCCGCCACGAACCCATCAACCACTCAGATATGGTGGACAAGAT
GTTTGGCTTCCTGGGGACTTCAGGCAGCCTGCCAGGCCAGGAAGGCCAGGCGCCTAGTGGCTTTGAGGACCTAGAGC
GCGGACGGAGGGAGATGGTGGAAGAGGATGTTGACGCTGCCCTGCCCCTGCCTGATGAAGACGAGGAGGACCTTTCT
GAGTACAAATTCGCCAAGTTTGCTGCCACCTACTTCCAGGGCACAACCACACACTCCTACACCCGGAGGCCTCTCAA
GCAGCCGCTGCTCTACCACGACGATGAGGGTGACCAGCTGGCGGCGCTGGCTGTCTGGATCACCATCCTCCGGTTCA
TGGGGGACCTCCCAGAGCCCAAGTACCACACAGCCATGAGCGACGGCAGTGAGAAGATCCCAGTGATGACTAAGATC
TACGAGACCCTAGGCAAGAAGACATATAAGAGGGAGCTGCAGGCCTTGCAGGGCGAGGGCGAGACCCAGCTCCCTGA
GGGGCAGAAGAAGACCAGTGTGAGACACAAGTTGGTACACTTGACACTGAAGAAAAAGTCCAAACTCACAGAAGAGG
TGACCAAGAGGCTGAACGATGGGGAATCCACGGTACAGGGCAACAGCATGCTGGAGGATCGGCCCACCTCAAATCTA
GAGAAGCTGCACTTCATCATCGGCAACGGCATCCTGCGGCCTGCGCTGCGGGACGAGATTTACTGCCAGATCAGTAA
GCAGCTCACACACAACCCATCCAAGAGCAGCTATGCCAGGGGCTGGATCCTCGTGTCGCTCTGTGTGGGCTGCTTCG
CCCCCTCTGAGAAGTTCGTTAAGTACCTGCGGAACTTCATCCACGGAGGCCCACCTGGCTATGCTCCTTACTGTGAG
GAGCGCCTGAGGAGGACCTTTGTCAACGGAACTCGGACACAGCCACCCAGCTGGCTGGAGCTGCAGGCCACCAAGTC
CAAGAAGCCCATCATGTTGCCCGTGACCTTCATGGATGGGACCACCAAGACCCTGCTAACAGATTCAGCAACTACAG
CCAGGGAGCTGTGCAATGCTCTGGCTGACAAGATCTCACTCAAGGACCGCTTTGGCTTCTCCCTCTACATCGCTCTG
TTCGATAAGGTGTCCTCCCTGGGCAGCGGCAGTGACCATGTCATGGATGCCATCTCTCAGTGTGAGCAGTACGCCAA
GGAGCAGGGTGCTCAGGAGCGCAACGCCCCATGGAGGCTCTTCTTTAGAAAGGAGGTCTTCACACCCTGGCACAACC
CCTCGGAGGACAACGTGGCCACGAACCTCATCTACCAGCAGGTGGTGCGAGGAGTCAAGTTTGGGGAGTACAGGTGT
GAGAAGGAGGACGACCTGGCTGAGCTGGCTTCTCAGCAGTACTTTGTGGACTATGGTTCTGAGATGATTCTGGAGCG
CCTGCTGAGCCTCGTGCCCACTTACATCCCTGACCGTGAGATCACACCGCTGAAGAATCTTGAGAAGTGGGCACAGC
TGGCCATTGCTGCCCACAAGAAGGGAATTTATGCCCAGAGGAGAACTGACTCCCAGAAGGTCAAAGAGGATGTGGTC
AATTATGCCCGTTTCAAGTGGCCCTTGCTCTTCTCCAGGTTTTACGAAGCTTACAAATTCTCAGGCCCTCCCCTCCC
CAAGAGCGACGTCATCGTGGCTGTCAACTGGACGGGTGTGTACTTCGTGGACGAGCAGGAGCAGGTGCTTCTGGAGC
TGTCCTTCCCGGAGATCATGGCTGTGTCCAGCAGTAGGGGAACAAAGATGATGGCCCCCAGCTTTACCCTGGCCACC
ATCAAAGGAGATGAGTACACCTTCACATCCAGCAATGCTGAGGACATCCGTGACCTGGTGGTCACCTTTCTGGAGGG
GCTACGGAAGAGGTCTAAGTATGTGGTGGCACTGCAGGACAATCCTAACCCTGCTGGTGAGGAGTCAGGCTTCCTCA
GCTTCGCCAAGGGAGACCTCATCATCCTTGACCATGATACTGGTGAGCAGGTCATGAACTCAGGCTGGGCCAACGGC
ATCAACGAGAGGACCAAGCAGCGCGGCGACTTCCCCACTGACTGTGTATACGTCATGCCCACTGTCACCTTGCCACC
AAGGGAGATTGTGGCCCTGGTCACTATGACCCCAGACCAGAGGCAGGATGTCGTCCGGCTCCTGCAGCTTCGCACAG
CAGAGCCAGAGGTGCGCGCCAAGCCCTACACGCTAGAGGAGTTCTCCTACGACTACTTCAGGCCCCCACCCAAGCAC
ACGCTGAGCCGTGTCATGGTGTCCAAGGCCCGCGGTAAGGACAGGCTGTGGAGCCACACACGAGAGCCCCTCAAGCA
GGCCCTGCTCAAGAAGATCCTGGGCAGTGAAGAACTCTCCCAGGAAGCCTGCATGGCCTTTGTAGCTGTGCTCAAGT
ACATGGGCGACTACCCATCCAAGAGGATGCGATCCGTCAATGAGCTCACTGACCAGATCTTTGAGTGGGCACTCAAG
GCTGAGCCCCTCAAGGATGAGGCCTACGTGCAGATCCTGAAGCAGCTGACTGACAATCACATCAGGTACAGCGAAGA
GAGGGGCTGGGAACTGCTGTGGCTGTGCACGGGCCTCTTCCCGCCCAGCAACATCCTCCTGCCTCATGTTCAGCGGT
TTCTGCAGTCCCGCAAGCACTGTCCTCTTGCCATTGACTGCCTGCAGAGGCTCCAGAAAGCCCTGAGAAATGGCTCC
CGGAAGTACCCTCCGCACCTGGTGGAGGTGGAGGCCATCCAACATAAGACTACCCAGATCTTCCACAAGGTCTACTT
CCCCGATGACACGGACGAGGCTTTTGAGGTGGAGTCCAGCACCAAGGCCAAGGACTTCTGCCAGAACATCGCCAGCC
GGCTGCTGCTCAAGTCTTCCGAGGGATTCAGCCTTTTTGTCAAAATCGCAGATAAGGTCATCAGCGTCCCAGAGAAT
GATTTCTTCTTTGACTTTGTCCGACACCTGACAGACTGGATAAAGAAAGCACGGCCCATCAAGGACGGAATCGTGCC
CTCACTAACCTACCAGGTGTTCTTCATGAAGAAGCTGTGGACCACCACAGTGCCGGGCAAGGACCCCATGGCTGACT
CCATCTTCCACTATTACCAGGAACTGCCCAAATATCTCCGAGGCTACCACAAGTGCACCCGGGAGGAGGTGCTGCAG
CTGGGCGCACTCATCTACAGGGTCAAGTTTGAGGAGGACAAATCCTACTTCCCTAGCATCCCCAAGTTGCTGAGGGA
GCTGGTACCCCAGGACCTAATCCGGCAGGTCTCACCTGATGACTGGAAACGGTCTATTGTCGCCTACTTCAACAAAC
ATGCGGGGAAGTCCAAGGAGGAAGCCAAGCTGGCCTTCCTCAAACTCATCTTCAAGTGGCCCACCTTTGGCTCAGCC
TTCTTTGAGGTGAAGCAAACTACAGAACCAAACTTCCCAGAGATTCTCTTAATTGCCATCAACAAGTACGGGGTCAG
CCTCATCGATCCCAGAACCAAGGACATCCTGACTACTCACCCCTTCACCAAGATCTCCAACTGGAGTAGTGGCAACA
CCTACTTCCACATCACCATTGGGAACTTGGTCCGTGGGAGCAAACTGCTCTGTGAGACATCGCTGGGATACAAAATG
GATGATCTTCTGACTTCCTACATCAGCCAGATGCTCACAGCCATGAGCAAGCAGAGGAACTCCAGGAGTGGAAGGTG
AACCTCAGAGGAGACGCTGGCTCAGGCCTTGGCCCTCTAGGCAGGGAAGCTGGACTGACCATATCTGCTGGGCATCT
GATCTGCCTGCCACGAGGTCCAGACTCTTCTGCATCCACCTATGGCATCTGGGTTTGCTGTCACCCTACTTTGTTGT
GGCCTTCCTGGTGTAAGAGTCTGTGTTCCTTGGTCACCTCTCCTGATTCAGACCAGCTCCATCAAGCAACCTCTTTT
GACTTTCTGTATATGGATGGCACAGAGGAATCAAGGACAACTTAGCTCTCTGCATACTTGGAACAACCAAACTATTT
GTACATTGAACGGATGCTCTGAAACCCAAGGGACTGGGCTCAGGGTCCTCAGCACTGGCCCCTGTCATAAGCACTAC
CACTAAGGACTCTCTGGAGGACTCCTCAGTATCATCTGCTCCAGGAAGCCCCCTAGACTACCTCCTGAGTCTGGACA
AAGCCTCCTGATTCTACCTGGATCACTCCTGTTATGTGACAGTTATGTGGTGGGTCCCTGCTAAAATCTCCCTGACC
ACCTGAGGGCATAAAGCATGTGTCTTATTCTCTGG (SEQ ID NO: 11)
Mus musculus myosin VIIA (Myo7a), isoform 4, protein
NCBI RefSeq NP_001243012.1
MDLKSGQEFDVPIGAVVKLCDSGQIQVVDDEDNEHWISPQNATHIKPMHPTSVHGVEDMIRLGDLNEAGILRNLLIR
YRDHLIYTYTGSILVAVNPYQLLSIYSPEHIRQYTNKKIGEMPPHIFAIADNCYFNMKRNNRDQCCIISGESGAGKT
ESTKLILQFLAAISGQHSWIEQQVLEATPILEAFGNAKTIRNDNSSRFGKYIDIHENKRGAIEGAKIEQYLLEKSRV
CRQAPDERNYHVFYCMLEGMNEEEKKKLGLGQAADYNYLAMGNCITCEGRVDSQEYANIRSAMKVLMFTDTENWEIS
KLLAAILHMGNLQYEARTFENLDACEVLFSPSLATAASLLEVNPPDLMSCLTSRTLITRGETVSTPLSREQALDVRD
AFVKGIYGRLFVWIVEKINAAIYKPPPLEVKNSRRSIGLLDIFGFENFTVNSFEQLCINFANEHLQQFFVRHVFKLE
QEEYDLESIDWLHIEFTDNQEALDMIANRPMNVISLIDEESKFPKGTDATMLHKLNSQHKLNANYVPPKNSHETQFG
INHFAGVVYYESQGFLEKNRDTLHGDIIQLVHSSRNKFIKQIFQADVAMGAETRKRSPTLSSQFKRSLELLMRTLGA
CQPFFVRCIKPNEFKKPMLFDRHLCVRQLRYSGMMETIRIRHAGYPIRYSFVEFVERYRVLLPGVKPAYKQGDLRGT
CQRMAEAVLGTHDDWQIGKTKIFLKDHHDMLLEVERDKAITDRVILLQKVIRGFKDRSNFLRLKSAATLIQRHWRGH
HCRKNYELIRLGFLRLQALHRSRKLHKQYRLARQRIIEFQARCRAYLVRKAFRHRLWAVITVQAYARGMIARRLHRR
LRVEYQRRLEAERMRLAEEEKLRKEMSAKKAKEEAERKHQERLAQLAREDAERELKEKEEARRKKELLEQMEKARHE
PINHSDMVDKMFGFLGTSGSLPGQEGQAPSGFEDLERGRREMVEEDVDAALPLPDEDEEDLSEYKFAKFAATYFQGT
TTHSYTRRPLKQPLLYHDDEGDQLAALAVWITILRFMGDLPEPKYHTAMSDGSEKIPVMTKIYETLGKKTYKRELQA
LQGEGETQLPEGQKKTSVRHKLVHLTLKKKSKLTEEVTKRLNDGESTVQGNSMLEDRPTSNLEKLHFIIGNGILRPA
LRDEIYCQISKQLTHNPSKSSYARGWILVSLCVGCFAPSEKFVKYLRNFIHGGPPGYAPYCEERLRRTFVNGTRTQP
PSWLELQATKSKKPIMLPVTFMDGTTKtLLTDSATTARELCNALADKISLKDRFGFSLYIALFDKVSSLGSGSDHVM
DAISQCEQYAKEQGAQERNAPWRLFFRKEVFTPWHNPSEDNVATNLIYQQVVRGVKFGEYRCEKEDDLAELASQQYF
VDYGSEMILERLLSLVPTYIPDREITPLKNLEKWAQLAIAAHKKGIYAQRRTDSQKVKEDVVNYARFKWPLLFSRFY
EAYKFSGPPLPKSDVIVAVNWTGVYFVDEQEQVLLELSFPEIMAVSSSRGTKMMAPSFTLATIKGDEYTFTSSNAED
IRDLVVTFLEGLRKRSKYVVALQDNPNPAGEESGFLSFAKGDLIILDHDTGEQVMNSGWANGINERTKQRGDFPTDC
VYVMPTVTLPPREIVALVTMTPDQRQDVVRLLQLRTAEPEVRAKPYTLEEFSYDYFRPPPKHTLSRVMVSKARGKDR
LWSHTREPLKQALLKKILGSEELSQEACMAFVAVLKYMGDYPSKRMRSVNELTDQIFEWALKAEPLKDEAYVQILKQ
LTDNHIRYSEERGWELLWLCTGLFPPSNILLPHVQRFLQSRKHCPLAIDCLQRLQKALRNGSRKYPPHLVEVEAIQH
KTTQIFHKVYFPDDTDEAFEVESSTKAKDFCQNIASRLLLKSSEGFSLFVKIADKVISVPENDFFFDFVRHLTDWIK
KARPIKDGIVPSLTYQVFFMKKLWTTTVPGKDPMADSIFHYYQELPKYLRGYHKCTREEVLQLGALIYRVKFEEDKS
YFPSIPKLLRELVPQDLIRQVSPDDWKRSIVAYFNKHAGKSKEEAKLAFLKLIFKWPTFGSAFFEVKQTTEPNFPEI
LLIAINKYGVSLIDPRTKDILTTHPFTKISNWSSGNTYFHITIGNLVRGSKLLCETSLGYKMDDLLTSYISQMLTAM
SKQRNSRSGR (SEQ ID NO: 12)
Mus musculus myosin VIIA (Myo7a), transcript variant 2, mRNA
NCBI RefSeq NM_008663.2
AGTGCAGGCTGGACAGCTGCCCTGAACAGAAAGAAAGAGTGACCCAGGGAGACAAGAAACAGAGTAGCCCAAGGGAA
GCCCACAGCAGCAGCAGATCAAGGCTCAAGCTGGAGCTGAAAATTTGCAGGCTCCAGCCTCAGCTTCCAGAGTCCTC
CTGACCTGTGACCCCTGGCTCCTGGCTGGGAGGTGGTGACTCGGAGGGTGTGGATAAAACCCAGAGCTGTGTCTGGT
CACTCCGGCAGGTGTGCTGACGTAGAAGCATGGTTATTCTGCAGAAGGGGGACTATGTATGGATGGACCTGAAGTCA
GGCCAGGAGTTTGATGTGCCCATCGGGGCCGTGGTGAAGCTCTGCGACTCGGGCCAGATCCAGGTGGTGGATGATGA
AGACAATGAACACTGGATATCCCCTCAGAATGCCACGCACATCAAGCCAATGCACCCCACATCGGTGCACGGCGTGG
AGGACATGATCCGCCTGGGGGATCTCAACGAGGCAGGCATCCTTCGAAACCTTCTCATTCGCTACCGGGACCACCTC
ATCTATACGTACACAGGTTCCATCCTGGTGGCCGTGAACCCCTACCAGCTGCTCTCCATCTACTCGCCAGAGCACAT
CCGCCAGTACACCAACAAGAAGATAGGGGAGATGCCCCCCCACATCTTCGCCATTGCTGACAACTGCTACTTCAACA
TGAAACGCAACAACCGGGACCAGTGCTGTATTATCAGCGGGGAGTCGGGAGCTGGCAAGACAGAGAGCACAAAGTTG
ATCCTGCAGTTCCTGGCAGCCATCAGTGGACAGCACTCATGGATCGAGCAGCAGGTGCTGGAGGCCACCCCGATCCT
GGAAGCATTTGGGAACGCCAAGACCATCCGCAACGACAACTCTAGCCGCTTTGGCAAGTACATTGACATCCACTTTA
ACAAGCGTGGTGCCATCGAGGGCGCCAAAATAGAGCAATACCTGCTGGAGAAGTCACGTGTCTGCCGCCAGGCCCCT
GACGAGAGGAACTATCACGTGTTCTACTGTATGCTGGAGGGCATGAATGAGGAGGAGAAGAAGAAACTGGGCCTAGG
CCAGGCCGCTGACTACAACTACTTGGCCATGGGTAACTGCATCACCTGTGAGGGCCGCGTGGACAGTCAGGAGTATG
CCAACATCCGCTCTGCCATGAAGGTTCTCATGTTCACAGACACGGAGAACTGGGAGATCTCGAAGCTTCTGGCTGCC
ATCCTACACATGGGCAATCTGCAGTATGAGGCCCGGACATTTGAGAACTTGGATGCGTGTGAAGTCCTCTTCTCCCC
ATCGCTGGCCACGGCAGCTTCTCTGCTCGAGGTGAACCCCCCAGACCTGATGAGCTGCCTCACCAGCCGCACCCTCA
TCACCCGTGGGGAGACGGTGTCCACCCCTCTCAGCAGGGAACAGGCGCTGGATGTGCGAGATGCCTTTGTCAAGGGC
ATCTATGGGCGGCTCTTTGTGTGGATTGTGGAGAAGATCAACGCAGCAATCTACAAGCCACCCCCCCTGGAAGTGAA
GAACTCTCGCCGGTCCATCGGTCTCCTGGACATCTTTGGATTTGAGAACTTCACTGTGAACAGCTTCGAGCAGCTCT
GCATTAACTTTGCCAATGAGCACCTGCAGCAATTCTTCGTGCGGCACGTGTTCAAGCTGGAGCAGGAGGAGTACGAC
CTGGAGAGCATCGACTGGTTGCACATTGAGTTCACTGACAACCAGGAAGCACTGGACATGATTGCCAACCGGCCTAT
GAACGTCATCTCCCTCATCGATGAGGAGAGCAAGTTCCCCAAGGGCACGGATGCCACCATGCTGCATAAGCTGAACT
CACAGCACAAGCTCAATGCCAACTACGTGCCACCCAAGAACAGCCACGAGACCCAGTTTGGAATCAACCACTTTGCG
GGTGTTGTCTATTATGAGAGTCAAGGCTTCCTGGAGAAGAACCGAGACACCCTGCATGGGGACATCATCCAGCTGGT
CCACTCTTCCCGGAACAAGTTCATAAAGCAGATTTTCCAAGCTGACGTTGCCATGGGTGCCGAGACCAGGAAGCGCT
CGCCTACACTCAGCAGCCAGTTCAAGCGGTCTCTGGAGCTGCTGATGCGCACACTGGGCGCCTGCCAGCCCTTCTTT
GTGCGTTGTATCAAACCCAATGAGTTCAAGAAGCCCATGCTCTTCGACCGGCACTTGTGTGTACGCCAGCTGCGATA
TTCGGGCATGATGGAGACAATCCGCATCCGCCACGCAGGCTACCCCATTCGCTACAGCTTTGTGGAGTTTGTGGAGC
GCTACCGGGTACTGCTGCCTGGTGTGAAGCCAGCATACAAGCAGGGTGACCTCCGAGGGACATGCCAGCGCATGGCT
GAGGCTGTGCTGGGCACGCACGATGACTGGCAGATTGGCAAAACCAAGATCTTTCTGAAGGACCACCATGACATGTT
GCTGGAGGTGGAGCGGGACAAGGCCATCACAGACAGAGTCATTCTCCTCCAGAAGGTTATCCGGGGCTTCAAAGACA
GGTCCAACTTCCTGAGACTGAAGAGTGCTGCCACACTGATCCAGAGGCACTGGCGGGGCCACCACTGTAGGAAAAAC
TATGAGCTGATTCGTCTTGGCTTCCTGCGGCTGCAGGCCCTGCACCGCTCCCGGAAGCTGCACAAGCAGTACCGCCT
GGCCAGACAGCGCATAATAGAGTTCCAGGCCCGCTGCCGGGCCTATCTGGTGCGCAAGGCCTTCCGCCACCGCCTCT
GGGCCGTGATCACCGTGCAGGCCTATGCCCGAGGCATGATTGCCCGCCGGCTACACCGCCGCCTCCGGGTTGAGTAC
CAGCGGCGCCTCGAGGCAGAGAGGATGCGTCTGGCAGAGGAGGAGAAACTCCGAAAGGAGATGAGTGCCAAGAAGGC
CAAAGAGGAGGCTGAGCGCAAGCATCAGGAGCGCCTGGCTCAGCTAGCCCGCGAGGATGCGGAGCGGGAACTGAAGG
AGAAGGAGGAGGCTCGGAGGAAGAAGGAACTGCTGGAGCAGATGGAGAAGGCCCGCCACGAACCCATCAACCACTCA
GATATGGTGGACAAGATGTTTGGCTTCCTGGGGACTTCAGGCAGCCTGCCAGGCCAGGAAGGCCAGGCGCCTAGTGG
CTTTGAGGACCTAGAGCGCGGACGGAGGGAGATGGTGGAAGAGGATGTTGACGCTGCCCTGCCCCTGCCTGATGAAG
ACGAGGAGGACCTTTCTGAGTACAAATTCGCCAAGTTTGCTGCCACCTACTTCCAGGGCACAACCACACACTCCTAC
ACCCGGAGGCCTCTCAAGCAGCCGCTGCTCTACCACGACGATGAGGGTGACCAGCTGGCGGCGCTGGCTGTCTGGAT
CACCATCCTCCGGTTCATGGGGGACCTCCCAGAGCCCAAGTACCACACAGCCATGAGCGACGGCAGTGAGAAGATCC
CAGTGATGACTAAGATCTACGAGACCCTAGGCAAGAAGACATATAAGAGGGAGCTGCAGGCCTTGCAGGGCGAGGGC
GAGACCCAGCTCCCTGAGGGGCAGAAGAAGACCAGTGTGAGACACAAGTTGGTACACTTGACACTGAAGAAAAAGTC
CAAACTCACAGAAGAGGTGACCAAGAGGCTGAACGATGGGGAATCCACGGTACAGGGCAACAGCATGCTGGAGGATC
GGCCCACCTCAAATCTAGAGAAGCTGCACTTCATCATCGGCAACGGCATCCTGCGGCCTGCGCTGCGGGACGAGATT
TACTGCCAGATCAGTAAGCAGCTCACACACAACCCATCCAAGAGCAGCTATGCCAGGGGCTGGATCCTCGTGTCGCT
CTGTGTGGGCTGCTTCGCCCCCTCTGAGAAGTTCGTTAAGTACCTGCGGAACTTCATCCACGGAGGCCCACCTGGCT
ATGCTCCTTACTGTGAGGAGCGCCTGAGGAGGACCTTTGTCAACGGAACTCGGACACAGCCACCCAGCTGGCTGGAG
CTGCAGGCCACCAAGTCCAAGAAGCCCATCATGTTGCCCGTGACCTTCATGGATGGGACCACCAAGACCCTGCTAAC
AGATTCAGCAACTACAGCCAGGGAGCTGTGCAATGCTCTGGCTGACAAGATCTCACTCAAGGACCGCTTTGGCTTCT
CCCTCTACATCGCTCTGTTCGATAAGGTGTCCTCCCTGGGCAGCGGCAGTGACCATGTCATGGATGCCATCTCTCAG
TGTGAGCAGTACGCCAAGGAGCAGGGTGCTCAGGAGCGCAACGCCCCATGGAGGCTCTTCTTTAGAAAGGAGGTCTT
CACACCCTGGCACAACCCCTCGGAGGACAACGTGGCCACGAACCTCATCTACCAGCAGGTGGTGCGAGGAGTCAAGT
TTGGGGAGTACAGGTGTGAGAAGGAGGACGACCTGGCTGAGCTGGCTTCTCAGCAGTACTTTGTGGACTATGGTTCT
GAGATGATTCTGGAGCGCCTGCTGAGCCTCGTGCCCACTTACATCCCTGACCGTGAGATCACACCGCTGAAGAATCT
TGAGAAGTGGGCACAGCTGGCCATTGCTGCCCACAAGAAGGGAATTTATGCCCAGAGGAGAACTGACTCCCAGAAGG
TCAAAGAGGATGTGGTCAATTATGCCCGTTTCAAGTGGCCCTTGCTCTTCTCCAGGTTTTACGAAGCTTACAAATTC
TCAGGCCCTCCCCTCCCCAAGAGCGACGTCATCGTGGCTGTCAACTGGACGGGTGTGTACTTCGTGGACGAGCAGGA
GCAGGTGCTTCTGGAGCTGTCCTTCCCGGAGATCATGGCTGTGTCCAGCAGTAGGGGAACAAAGATGATGGCCCCCA
GCTTTACCCTGGCCACCATCAAAGGAGATGAGTACACCTTCACATCCAGCAATGCTGAGGACATCCGTGACCTGGTG
GTCACCTTTCTGGAGGGGCTACGGAAGAGGTCTAAGTATGTGGTGGCACTGCAGGACAATCCTAACCCTGCTGGTGA
GGAGTCAGGCTTCCTCAGCTTCGCCAAGGGAGACCTCATCATCCTTGACCATGATACTGGTGAGCAGGTCATGAACT
CAGGCTGGGCCAACGGCATCAACGAGAGGACCAAGCAGCGCGGCGACTTCCCCACTGACTGTGTATACGTCATGCCC
ACTGTCACCTTGCCACCAAGGGAGATTGTGGCCCTGGTCACTATGACCCCAGACCAGAGGCAGGATGTCGTCCGGCT
CCTGCAGCTTCGCACAGCAGAGCCAGAGGTGCGCGCCAAGCCCTACACGCTAGAGGAGTTCTCCTACGACTACTTCA
GGCCCCCACCCAAGCACACGCTGAGCCGTGTCATGGTGTCCAAGGCCCGCGGTAAGGACAGGCTGTGGAGCCACACA
CGAGAGCCCCTCAAGCAGGCCCTGCTCAAGAAGATCCTGGGCAGTGAAGAACTCTCCCAGGAAGCCTGCATGGCCTT
TGTAGCTGTGCTCAAGTACATGGGCGACTACCCATCCAAGAGGATGCGATCCGTCAATGAGCTCACTGACCAGATCT
TTGAGTGGGCACTCAAGGCTGAGCCCCTCAAGGATGAGGCCTACGTGCAGATCCTGAAGCAGCTGACTGACAATCAC
ATCAGGTACAGCGAAGAGAGGGGCTGGGAACTGCTGTGGCTGTGCACGGGCCTCTTCCCGCCCAGCAACATCCTCCT
GCCTCATGTTCAGCGGTTTCTGCAGTCCCGCAAGCACTGTCCTCTTGCCATTGACTGCCTGCAGAGGCTCCAGAAAG
CCCTGAGAAATGGCTCCCGGAAGTACCCTCCGCACCTGGTGGAGGTGGAGGCCATCCAACATAAGACTACCCAGATC
TTCCACAAGGTCTACTTCCCCGATGACACGGACGAGGCTTTTGAGGTGGAGTCCAGCACCAAGGCCAAGGACTTCTG
CCAGAACATCGCCAGCCGGCTGCTGCTCAAGTCTTCCGAGGGATTCAGCCTTTTTGTCAAAATCGCAGATAAGGTCA
TCAGCGTCCCAGAGAATGATTTCTTCTTTGACTTTGTCCGACACCTGACAGACTGGATAAAGAAAGCACGGCCCATC
AAGGACGGAATCGTGCCCTCACTAACCTACCAGGTGTTCTTCATGAAGAAGCTGTGGACCACCACAGTGCCGGGCAA
GGACCCCATGGCTGACTCCATCTTCCACTATTACCAGGAACTGCCCAAATATCTCCGAGGCTACCACAAGTGCACCC
GGGAGGAGGTGCTGCAGCTGGGCGCACTCATCTACAGGGTCAAGTTTGAGGAGGACAAATCCTACTTCCCTAGCATC
CCCAAGTTGCTGAGGGAGCTGGTACCCCAGGACCTAATCCGGCAGGTCTCACCTGATGACTGGAAACGGTCTATTGT
CGCCTACTTCAACAAACATGCGGGGAAGTCCAAGGAGGAAGCCAAGCTGGCCTTCCTCAAACTCATCTTCAAGTGGC
CCACCTTTGGCTCAGCCTTCTTTGAGGTGAAGCAAACTACAGAACCAAACTTCCCAGAGATTCTCTTAATTGCCATC
AACAAGTACGGGGTCAGCCTCATCGATCCCAGAACCAAGGACATCCTGACTACTCACCCCTTCACCAAGATCTCCAA
CTGGAGTAGTGGCAACACCTACTTCCACATCACCATTGGGAACTTGGTCCGTGGGAGCAAACTGCTCTGTGAGACAT
CGCTGGGATACAAAATGGATGATCTTCTGACTTCCTACATCAGCCAGATGCTCACAGCCATGAGCAAGCAGAGGAAC
TCCAGGAGTGGAAGGTGAACCTCAGAGGAGACGCTGGCTCAGGCCTTGGCCCTCTAGGCAGGGAAGCTGGACTGACC
ATATCTGCTGGGCATCTGATCTGCCTGCCACGAGGTCCAGACTCTTCTGCATCCACCTATGGCATCTGGGTTTGCTG
TCACCCTACTTTGTTGTGGCCTTCCTGGTGTAAGAGTCTGTGTTCCTTGGTCACCTCTCCTGATTCAGACCAGCTCC
ATCAAGCAACCTCTTTTGACTTTCTGTATATGGATGGCACAGAGGAATCAAGGACAACTTAGCTCTCTGCATACTTG
GAACAACCAAACTATTTGTACATTGAACGGATGCTCTGAAACCCAAGGGACTGGGCTCAGGGTCCTCAGCACTGGCC
CCTGTCATAAGCACTACCACTAAGGACTCTCTGGAGGACTCCTCAGTATCATCTGCTCCAGGAAGCCCCCTAGACTA
CCTCCTGAGTCTGGACAAAGCCTCCTGATTCTACCTGGATCACTCCTGTTATGTGACAGTTATGTGGTGGGTCCCTG
CTAAAATCTCCCTGACCACCTGAGGGCATAAAGCATGTGTCTTATT (SEQ ID NO: 13)
Mus musculus myosin VIIA (Myo7a), isoform 2, protein
NCBI RefSeq NP_032689.2
MVILQKGDYVWMDLKSGQEFDVPIGAVVKLCDSGQIQVVDDEDNEHWISPQNATHIKPMHPTSVHGVEDMIRLGDLN
EAGILRNLLIRYRDHLIYTYTGSILVAVNPYQLLSIYSPEHIRQYTNKKIGEMPPHIFAIADNCYFNMKRNNRDQCC
IISGESGAGKTESTKLILQFLAAISGQHSWIEQQVLEATPILEAFGNAKTIRNDNSSRFGKYIDIHFNKRGAIEGAK
IEQYLLEKSRVCRQAPDERNYHVFYCMLEGMNEEEKKKLGLGQAADYNYLAMGNCITCEGRVDSQEYANIRSAMKVL
MFTDTENWEISKLLAAILHMGNLQYEARTFENLDACEVLFSPSLATAASLLEVNPPDLMSCLTSRTLITRGETVSTP
LSREQALDVRDAFVKGIYGRLFVWIVEKINAAIYKPPPLEVKNSRRSIGLLDIFGFENFTVNSFEQLCINFANEHLQ
QFFVRHVFKLEQEEYDLESIDWLHIEFTDNQEALDMIANRPMNVISLIDEESKFPKGTDATMLHKLNSQHKLNANYV
PPKNSHETQFGINHFAGVVYYESQGFLEKNRDTLHGDIIQLVHSSRNKFIKQIFQADVAMGAETRKRSPTLSSQFKR
SLELLMRTLGACQPFFVRCIKPNEFKKPMLFDRHLCVRQLRYSGMMETIRIRHAGYPIRYSFVEFVERYRVLLPGVK
PAYKQGDLRGTCQRMAEAVLGTHDDWQIGKTKIFLKDHHDMLLEVERDKAITDRVILLQKVIRGFKDRSNFLRLKSA
ATLIQRHWRGHHCRKNYELIRLGFLRLQALHRSRKLHKQYRLARQRIIEFQARCRAYLVRKAFRHRLWAVITVQAYA
RGMIARRLHRRLRVEYQRRLEAERMRLAEEEKLRKEMSAKKAKEEAERKHQERLAQLAREDAERELKEKEEARRKKE
LLEQMEKARHEPINHSDMVDKMFGFLGTSGSLPGQEGQAPSGFEDLERGRREMVEEDVDAALPLPDEDEEDLSEYKF
AKFAATYFQGTTTHSYTRRPLKQPLLYHDDEGDQLAALAVWITILRFMGDLPEPKYHTAMSDGSEKIPVMTKIYETL
GKKTYKRELQALQGEGETQLPEGQKKTSVRHKLVHLTLKKKSKLTEEVTKRLNDGESTVQGNSMLEDRPTSNLEKLH
FIIGNGILRPALRDEIYCQISKQLTHNPSKSSYARGWILVSLCVGCFAPSEKFVKYLRNFIHGGPPGYAPYCEERLR
RTFVNGTRTQPPSWLELQATKSKKPIMLPVTFMDGTTKTLLTDSATTARELCNALADKISLKDRFGFSLYIALFDKV
SSLGSGSDHVMDAISQCEQYAKEQGAQERNAPWRLFFRKEVFTPWHNPSEDNVATNLIYQQVVRGVKFGEYRCEKED
DLAELASQQYFVDYGSEMILERLLSLVPTYIPDREITPLKNLEKWAQLAIAAHKKGIYAQRRTDSQKVKEDVVNYAR
FKWPLLFSRFYEAYKFSGPPLPKSDVIVAVNWTGVYFVDEQEQVLLELSFPEIMAVSSSRGTKMMAPSFTLATIKGD
EYTFTSSNAEDIRDLVVTFLEGLRKRSKYVVALQDNPNPAGEESGFLSFAKGDLIILDHDTGEQVMNSGWANGINER
TKQRGDFPTDCVYVMPTVTLPPREIVALVTMTPDQRQDVVRLLQLRTAEPEVRAKPYTLEEFSYDYFRPPPKHTLSR
VMVSKARGKDRLWSHTREPLKQALLKKILGSEELSQEACMAFVAVLKYMGDYPSKRMRSVNELTDQIFEWALKAEPL
KDEAYVQILKQLTDNHIRYSEERGWELLWLCTGLFPPSNILLPHVQRFLQSRKHCPLAIDCLQRLQKALRNGSRKYP
PHLVEVEAIQHKTTQIFHKVYFPDDTDEAFEVESSTKAKDFCQNIASRLLLKSSEGFSLFVKIADKVISVPENDFFF
DFVRHLTDWIKKARPIKDGIVPSLTYQVFFMKKLWTTTVPGKDPMADSIFHYYQELPKYLRGYHKCTREEVLQLGAL
IYRVKFEEDKSYFPSIPKLLRELVPQDLIRQVSPDDWKRSIVAYFNKHAGKSKEEAKLAFLKLIFKWPTFGSAFFEV
KQTTEPNFPEILLIAINKYGVSLIDPRTKDILTTHPFTKISNWSSGNTYFHITIGNLVRGSKLLCETSLGYKMDDLL
TSYISQMLTAMSKQRNSRSGR (SEQ ID NO: 14)

What is claimed is:

1. A method comprising providing to a subject a CRISPR-associated endonuclease, a guide RNA (gRNA), and a template nucleic acid, wherein the gRNA targets a MYO7A gene.

2. The method of claim 1, wherein the CRISPR-associated endonuclease is Cas9.

3. The method of claim 1 or 2, wherein the CRISPR-associated endonuclease is provided as a protein.

4. The method of any preceding claim, wherein the CRISPR-associated endonuclease is provided as a nucleic acid encoding a protein.

5. The method of claim 4, wherein the nucleic acid is a messenger RNA (mRNA).

6. The method of any preceding claim, wherein the CRISPR-associated endonuclease and the gRNA are provided as a ribonucleoprotein (RNP) complex or a nucleic acid encoding an RNP complex.

7. The method of any preceding claim, wherein the template nucleic acid comprises a portion of a nucleic acid sequence encoding a wild-type MYO7A protein or a sequence capable of specifically binding to a portion of a nucleic acid sequence encoding a wild-type MYO7A protein.

8. The method of claim 7, wherein the wild-type MYO7A protein is a mammalian MYO7A protein.

9. The method of claim 7, wherein the wild-type MYO7A protein is a human MYO7A protein.

10. The method of claim 7, wherein the wild-type MYO7A protein is a mouse MYO7A protein.

11. The method of any preceding claim, wherein the gRNA comprises, consists essentially of, or consists of a nucleic acid sequence of 10-30 or 15-25 consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13), or a nucleotide sequence of 10-30 or 15-25 nucleotides capable of specifically hybridizing to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13).

12. The method of any preceding claim, wherein the gRNA comprises, consists essentially of, or consists of a nucleic acid sequence of, or capable of specifically binding to any one of the sequences of
(SEQ ID NO: 16)
GATGACGTTCATAGGCCGGTTGG,
(SEQ ID NO: 17)
CTTGCTCTCCTCATCGATGAGGG,
(SEQ ID NO: 18)
ATGAGGGAGATGACGTTCATAGG,
(SEQ ID NO: 19)
AGGGAGATGACGTTCATAGGCGG,
(SEQ ID NO: 20)
CAATCATGTCCAGTGCTTCCTGG,
(SEQ ID NO: 40)
GAUGACGUUCAUAGGCGGGU,
(SEQ ID NO: 41)
GACGUUCAUAGGCGGGU,
(SEQ ID NO: 42)
AGGGAGAUGACGUUCAUAGG,
(SEQ ID NO: 43)
GAGAUGACGUUCAUAGG,
(SEQ ID NO: 44)
CUUGCUCUCCUCAUCGAUGA,
or
(SEQ ID NO: 45)
AUGAGGGAGAUGACGUUCAU,
wherein each uracil base (U) may independently and optionally be replaced with a thymine base (T) and each T may independently and optionally be replaced with a U.

13. The method of any one of claims 1-9, wherein the gRNA comprises, consists essentially of, or consists of a nucleotide sequence of 10-30 or 15-25 consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5) or a nucleotide sequence of 10-30 or 15-25 nucleotides capable of specifically hybridizing to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5).

14. The method of any one of claims 1-12, wherein the MYO7A gene is a mouse MYO7A gene.

15. The method of any one of claim 1-9 or 13, wherein the MYO7A gene is a human MYO7A gene.

16. The method of any preceding claim, wherein the CRISPR-associated endonuclease, the gRNA, and/or the template nucleic acid are encapsulated within an extracellular vesicle.

17. The method of claim 16, wherein the extracellular vesicle is an exosome.

18. The method of claim 16 or 17, wherein the extracellular vesicle is isolated or derived from an auditory cell, optionally wherein the auditory cell is an HEI-OC1 cell.

19. A composition comprising a CRISPR-associated endonuclease or a nucleic acid sequence encoding a CRISPR-associated endonuclease, a guide RNA (gRNA), and a template nucleic acid, wherein the gRNA is targets a MYO7A gene.

20. The composition of claim 19, comprised within an extracellular vesicle.

21. The composition of claim 20, wherein the extracellular vesicle is an exosome.

22. The composition of claim 20 or 21, wherein the extracellular vesicle is isolated or derived from an auditory cell, optionally wherein the auditory cell is an HEI-OC1 cell.

23. The composition of any one of claims 19-22, further comprising a stabilizing agent.

24. The composition of claim 23, wherein the stabilizing agent is a disaccharide.

25. The composition of claim 23 or 24, wherein the stabilizing agent is trehalose.

26. The composition of any one of claims 20-25, wherein the stabilizing agent is associated with the extracellular vesicle.

27. The composition of any one of claims 19-26, wherein the CRISPR-associated endonuclease is Cas9.

28. The composition of any one of claims 19-27, comprising a CRISPR-associated endonuclease.

29. The composition of any one of claims 19-27, comprising a nucleic acid encoding a CRISPR-associated endonuclease.

30. The composition of any one of claims 19-29, wherein the template nucleic acid comprises a portion of a nucleic acid sequence encoding a wild-type MYO7A protein.

31. The composition of any one of claims 19-30, wherein the gRNA comprises, consists essentially of, or consists of a nucleic acid sequence of 10-30 or 15-25 consecutive nucleotides of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13), or a nucleotide sequence of 10-30 or 15-25 nucleotides capable of specifically hybridizing to an equal-length portion of the sequence of NCBI Reference Sequence NM_001256081.1 (SEQ ID NO: 7), NM_001256082.1 (SEQ ID NO: 9), NM_001256083.1 (SEQ ID NO: 11), or NM_008663.2 (SEQ ID NO: 13).

32. The composition of any one of claims 19-31, wherein the gRNA comprises, consists essentially of, or consists of a nucleic acid sequence of, or capable of specifically binding to any one of the sequences of
(SEQ ID NO: 16)
GATGACGTTCATAGGCCGGTTGG,
(SEQ ID NO: 17)
CTTGCTCTCCTCATCGATGAGGG,
(SEQ ID NO: 18)
ATGAGGGAGATGACGTTCATAGG,
(SEQ ID NO: 19)
AGGGAGATGACGTTCATAGGCGG,
(SEQ ID NO: 20)
CAATCATGTCCAGTGCTTCCTGG,
(SEQ ID NO: 40)
GAUGACGUUCAUAGGCGGGU,
(SEQ ID NO: 41)
GACGUUCAUAGGCGGGU,
(SEQ ID NO: 42)
AGGGAGAUGACGUUCAUAGG,
(SEQ ID NO: 43)
GAGAUGACGUUCAUAGG,
(SEQ ID NO: 44)
CUUGCUCUCCUCAUCGAUGA,
or
(SEQ ID NO: 45)
AUGAGGGAGAUGACGUUCAU,
wherein each uracil base (U) may independently and optionally be replaced with a thymine base (T) and each T may independently and optionally be replaced with a U.

33. The composition of any one of claims 19-30, wherein the gRNA comprises, consists essentially of, or consists of a nucleotide sequence of 10-30 or 15-25 consecutive nucleotides of the sequence of NCBI Reference Sequence NM_000260.4 (SEQ ID NO: 1), NM_001127180.2 (SEQ ID NO: 3), or NM_001369365.1 (SEQ ID NO: 5) or a nucleotide sequence of 10-30 or 15-25 nucleotides capable of specifically hybridizing to an equal-length portion of the sequence of NCBI Reference Sequence NM_000260.4 (SEQ ID NO: 1), NM_001127180.2 (SEQ ID NO: 3), or NM_001369365.1 (SEQ ID NO: 5).

34. The composition of any one of claims 19-32, wherein the MYO7A gene is a mouse MYO7A gene.

35. The composition of any one of claim 19-30 or 33, wherein the MYO7A gene is a human MYO7A gene.

36. A method of treating a hearing loss disorder, the method comprising administering to a subject in need thereof a composition of any one of claims 19-35 in an amount sufficient to treat a hearing loss disorder in the subject.

37. The method of claim 36, wherein the subject is a mammal, optionally wherein the mammal is a primate.

38. The method of claim 36 or 37, wherein the subject is a human.
